(19) United States
(12) Patent Application Publication
(10) Pub. No.: US 2012/0027782 A1
(43) Pub. Date: Feb. 2, 2012

(54) MONOCLONAL ANTIBODY PARTNER MOLECULE CONJUGATES DIRECTED TO PROTEIN TYROSINE KINASE 7 (PTK7)

(75) Inventors: Jonathan Alexander Terrett, Sunnyvale, CA (US); Chin Pan, Los Altos, CA (US); Chetana Rao-Naik, Walnut Creek, CA (US)

(73) Assignee: BRISTOL-MYERS SQUIBB COMPANY, Princeton, NJ (US)

(21) Appl. No.: 12/745,503

(22) PCT Filed: Nov. 26, 2008
(86) PCT No.: PCT/US08/84949

§ 371 Date: May 28, 2010

Related U.S. Application Data
(60) Provisional application No. 61/005,034, filed on Nov. 30, 2007.

(51) Int. Cl. A61K 39/44; A61P 35/00; C12N 15/63; C12N 5/10; C07K 17/02; C12N 15/13

(52) U.S. Cl. 424/178.1; 530/391.7; 536/23.53; 435/320.1; 530/391.9; 435/345

(57) ABSTRACT

The present disclosure relates to antibody-partner molecule conjugates directed to PTK7. Also described are methods for treating or preventing a disease characterized by growth of tumor cells expressing PTK7 using the antibody-partner molecule conjugates.

### BACKGROUND OF THE INVENTION

Receptor tyrosine kinases (RTKs) are transmembrane signaling proteins that transmit biological signals from the extracellular environment to the interior of the cell. The regulation of RTK signals is important for regulation of cell growth, differentiation, axonal growth, epithelial growth, development, adhesion, migration, and apoptosis (Prenzel et al. (2001) Endocr. Relat. Cancer 8:11-31; Hubbard and Till (2000) Annu. Rev. Biochem.

69:373-98). RTKs are known to be involved in the development and progression of several forms of cancer. In most of the RTK-related cancers, there has been an amplification of the receptor protein rather than a mutation of the gene (Kobus and Fleming (2005) Biochemistry 44:1464-70).

Protein tyrosine kinase 7 (PTK7), a member of the receptor protein tyrosine kinase family, was first isolated from normal human melanocytes and cloned by RT-PCR (Lee et al., (1993) Oncogene 8:3403-10; Park et al., (1996) J. Biochem 119:235-9). Separately, the gene was cloned from human colon carcinoma-derived cell lines and named colon carcinoma kinase 4 (CCK4) (Mossie et al. (1995) Oncogene 11:2179-84). PTK7 belongs to a subset of RTKs that lack detectable catalytic tyrosine kinase activity but retain signal transduction activity and is thought to possibly function as a cell adhesion molecule.

The mRNA for PTK7 was found to be variably expressed in colon carcinoma derived cell lines but not found to be expressed in human adult colon tissues (Mossie et al., supra). PTK7 expression was also seen in some melanoma cell lines and melanoma biopsies (Easty, et al. (1997) Int. J. Cancer 71:1061-5). An alternative splice form was found to be expressed in hepatomas and colon cancer cells (Jung et al. (2002) Biochim Biophys Acta 1579: 153-63). In addition, PTK7 was found to be highly overexpressed in acute myeloid leukemia samples (Muller-Tidow et al., (2004) Clin. Cancer Res. 10:1241-9). By immunohistochemistry, tumor specific staining of PTK7 was observed in breast, colon, lung, pancreatic, kidney and bladder cancers, as described in PCT Publication WO 04/17992.

Accordingly, agents that recognize PTK7, and methods of using such agents, are desired.

### SUMMARY OF THE INVENTION

The present invention provides isolated monoclonal antibodies, in particular human monoclonal antibodies, that bind to PTK7 and that exhibit numerous desirable properties. These properties include high affinity binding to human PTK7 and binding to Wilms' tumor cells. Also provided are methods for treating a variety of PTK7 mediated diseases using the antibodies and compositions of the invention. In one aspect, the invention pertains to an isolated monoclonal antibody, or an antigen-binding portion thereof, wherein the antibody:

(a) specifically binds to human PTK7; and

(b) binds to a Wilms' tumor cell line (ATCC Acc No. CRL-1441).

Preferably the antibody is a human antibody, although in alternative embodiments the antibody can be a murine antibody, a chimeric antibody or humanized antibody.

In more preferred embodiments, the antibody binds to Wilms' tumor cells with an EC₅₀ of 4.0 nM or less or binds to Wilms' tumor cells with an EC₅₀ of 3.5 nM or less.

In another embodiment, the antibody binds to a cancer cell line selected from the group consisting of A-431 (ATCC Acc No. CRL-1555), Saos-2 (ATCC Acc No. HTB-85), SKOV-3 (ATCC Acc No. HTB-77), PC3 (ATCC Acc No. CRL-1435), DMS 114 (ATCC Acc No. CRL-2066), ACHN (ATCC Acc No. CRL-1611), LNCaP (ATCC Acc No. CRL-1740), DU 145 (ATCC Acc No. HTB-81), LoVo (ATCC Acc No. CCL-229) and MIA PaCa-2 (ATCC Acc No. CRL-1420) cell lines.

In another embodiment, the invention provides an isolated monoclonal antibody, or antigen binding portion thereof, wherein the antibody cross-competes for binding to an epitope on PTK7 which is recognized by a reference antibody, wherein the reference antibody:
(a) specifically binds to human PTK7; and
(b) binds to a Wilms' tumor cell line (ATCC Acc No. CRL-1441).
   In various embodiments, the reference antibody comprises:
(a) a heavy chain variable region comprising the amino acid sequence of SEQ ID NO:1; and
(b) a light chain variable region comprising the amino acid sequence of SEQ ID NO:5;
   or the reference antibody comprises:
(a) a heavy chain variable region comprising the amino acid sequence of SEQ ID NO:1; and
(b) a light chain variable region comprising the amino acid sequence of SEQ ID NO:6;
   or the reference antibody comprises:
(a) a heavy chain variable region comprising the amino acid sequence of SEQ ID NO:2; and
(b) a light chain variable region comprising the amino acid sequence of SEQ ID NO:7;
   or the reference antibody comprises:
(a) a heavy chain variable region comprising the amino acid sequence of SEQ ID NO:3; and
(b) a light chain variable region comprising the amino acid sequence of SEQ ID NO:8;
   or the reference antibody comprises:
(a) a heavy chain variable region comprising the amino acid sequence of SEQ ID NO:3; and
(b) a light chain variable region comprising the amino acid sequence of SEQ ID NO:9;
   or the reference antibody comprises:
(a) a heavy chain variable region comprising the amino acid sequence of SEQ ID NO:4; and
(b) a light chain variable region comprising the amino acid sequence of SEQ ID NO:10.

In one aspect, the invention pertains to an isolated monoclonal antibody, or an antigen-binding portion thereof, comprising a heavy chain variable region that is the product of or derived from a human V_{H} 3-30.3 gene, wherein the antibody specifically binds PTK7. The invention also provides an isolated monoclonal antibody, or an antigen-binding portion thereof, comprising a heavy chain variable region that is the product of or derived from a human V_{H} DP44 gene, wherein the antibody specifically binds PTK7. The invention also provides an isolated monoclonal antibody, or an antigen-binding portion thereof, comprising a heavy chain variable region that is the product of or derived from a human V_{H} 3-33 gene, wherein the antibody specifically binds PTK7. The invention further provides an isolated monoclonal antibody, or an antigen-binding portion thereof, comprising a light chain variable region that is the product of or derived from a human V_{K} L15 gene, wherein the antibody specifically binds PTK7. The invention further provides an isolated monoclonal antibody, or an antigen-binding portion thereof, comprising a light chain variable region that is the product of or derived from a human V_{K} A10 gene, wherein the antibody specifically binds PTK7. The invention further provides an isolated monoclonal antibody, or an antigen-binding portion thereof, comprising a light chain variable region that is the product of or derived from a human V_{K} A27 gene, wherein the antibody specifically binds PTK7. The invention further provides an isolated monoclonal antibody, or an antigen-binding portion thereof, comprising a light chain variable region that is the product of or derived from a human V_{K} L6 gene, wherein the antibody specifically binds PTK7.

A preferred combination comprises:
(a) a heavy chain variable region CDR1 comprising SEQ ID NO:11;
(b) a heavy chain variable region CDR2 comprising SEQ ID NO:15;
(c) a heavy chain variable region CDR3 comprising SEQ ID NO:19;
(d) a light chain variable region CDR1 comprising SEQ ID NO:23;
(e) a light chain variable region CDR2 comprising SEQ ID NO:29; and
(f) a light chain variable region CDR3 comprising SEQ ID NO:35.
   Another preferred combination comprises:
(a) a heavy chain variable region CDR1 comprising SEQ ID NO:11;
(b) a heavy chain variable region CDR2 comprising SEQ ID NO:15;
(c) a heavy chain variable region CDR3 comprising SEQ ID NO:19;
(d) a light chain variable region CDR1 comprising SEQ ID NO:24;
(e) a light chain variable region CDR2 comprising SEQ ID NO:30; and
(f) a light chain variable region CDR3 comprising SEQ ID NO:36.
   Another preferred combination comprises:
(a) a heavy chain variable region CDR1 comprising SEQ ID NO:12;
(b) a heavy chain variable region CDR2 comprising SEQ ID NO:16;
(c) a heavy chain variable region CDR3 comprising SEQ ID NO:20;
(d) a light chain variable region CDR1 comprising SEQ ID NO:25;
(e) a light chain variable region CDR2 comprising SEQ ID NO:31; and
(f) a light chain variable region CDR3 comprising SEQ ID NO:37.
   Another preferred combination comprises:
(a) a heavy chain variable region CDR1 comprising SEQ ID NO:13;
(b) a heavy chain variable region CDR2 comprising SEQ ID NO:17;
(c) a heavy chain variable region CDR3 comprising SEQ ID NO:21;
(d) a light chain variable region CDR1 comprising SEQ ID NO:26;
(e) a light chain variable region CDR2 comprising SEQ ID NO:32; and
(f) a light chain variable region CDR3 comprising SEQ ID NO:38.
   Another preferred combination comprises:
(a) a heavy chain variable region CDR1 comprising SEQ ID NO:13;
(b) a heavy chain variable region CDR2 comprising SEQ ID NO:17;
(c) a heavy chain variable region CDR3 comprising SEQ ID NO:21;
(d) a light chain variable region CDR1 comprising SEQ ID NO:27;
(e) a light chain variable region CDR2 comprising SEQ ID NO:33; and
(f) a light chain variable region CDR3 comprising SEQ ID NO:39.
   Another preferred combination comprises:
(a) a heavy chain variable region CDR1 comprising SEQ ID NO:14;
(b) a heavy chain variable region CDR2 comprising SEQ ID NO:18;
(c) a heavy chain variable region CDR3 comprising SEQ ID NO:22;
(d) a light chain variable region CDR1 comprising SEQ ID NO:28;
(e) a light chain variable region CDR2 comprising SEQ ID NO:34; and
(f) a light chain variable region CDR3 comprising SEQ ID NO:40.
   Other preferred antibodies of the invention, or antigen binding portions thereof comprise:
(a) a heavy chain variable region comprising the amino acid sequence of SEQ ID NO:1; and
(b) a light chain variable region comprising the amino acid sequence of SEQ ID NO:5.
   Another preferred combination comprises:
(a) a heavy chain variable region comprising the amino acid sequence of SEQ ID NO:1; and
(b) a light chain variable region comprising the amino acid sequence of SEQ ID NO:6.
   Another preferred combination comprises:
(a) a heavy chain variable region comprising the amino acid sequence of SEQ ID NO:2; and
(b) a light chain variable region comprising the amino acid sequence of SEQ ID NO:7.
   Another preferred combination comprises:
(a) a heavy chain variable region comprising the amino acid sequence of SEQ ID NO:3; and
(b) a light chain variable region comprising the amino acid sequence of SEQ ID NO:8.
   Another preferred combination comprises:
(a) a heavy chain variable region comprising the amino acid sequence of SEQ ID NO:3; and
(b) a light chain variable region comprising the amino acid sequence of SEQ ID NO:9.
   Another preferred combination comprises:
(a) a heavy chain variable region comprising the amino acid sequence of SEQ ID NO:4; and
(b) a light chain variable region comprising the amino acid sequence of SEQ ID NO:10.

The antibodies of the invention can be, for example, full-length antibodies, for example of an IgG1 or IgG4 isotype. Alternatively, the antibodies can be antibody fragments, such as Fab or Fab′2 fragments, or single chain antibodies.

The invention also provides an antibody-partner molecule conjugate comprising an antibody of the invention, or antigen-binding portion thereof, linked to a therapeutic agent, such as a cytotoxin or a radioactive isotope. In a particularly preferred embodiment, the invention provides an antibody-partner molecule conjugate comprising an antibody of this disclosure, or antigen-binding portion thereof, linked (e.g., via a thiol linkage) to compound N (FIG. 28). For example, in various embodiments, the invention provides the following preferred antibody-partner molecule conjugates:

(i) an antibody-partner molecule conjugate comprising an antibody, or antigen-binding portion thereof, comprising a heavy chain variable region comprising the amino acid sequence of SEQ ID NO:4; and a light chain variable region comprising the amino acid sequence of SEQ ID NO:10, where the antibody or antigen-binding portion thereof is linked to compound N (FIG. 28), which is discussed in detail in U.S. Patent App. No. 60/882,461, which is hereby incorporated by reference in its entirety;

(ii) an antibody-partner molecule conjugate comprising an antibody, or antigen-binding portion thereof, comprising:
(a) a heavy chain variable region CDR1 comprising SEQ ID NO:14;
(b) a heavy chain variable region CDR2 comprising SEQ ID NO:18;
(c) a heavy chain variable region CDR3 comprising SEQ ID NO:22;
(d) a light chain variable region CDR1 comprising SEQ ID NO:28;
(e) a light chain variable region CDR2 comprising SEQ ID NO:34; and
(f) a light chain variable region CDR3 comprising SEQ ID NO:40;
   linked to compound N (FIG. 28); and

In a particular embodiment, the antibody, or antigen binding portion thereof, of the antibody-partner molecule conjugate has heavy and light chain variable regions comprising the amino acid sequences set forth in SEQ ID NOs:4 and 10, SEQ ID NOs:1 and 5, SEQ ID NOs:1 and 6, SEQ ID NOs:2 and 7, SEQ ID NOs:3 and 8, or SEQ ID NOs:3 and 9, respectively.

Also provided by the present invention are antibody-partner molecule conjugates, wherein the antibody binds to the same or overlapping epitopes bound by any of the antibodies of the present invention. For example, in one embodiment, the antibody, or antigen binding portion thereof, of the antibody-partner molecule conjugate binds to an epitope on PTK-7 recognized by a reference antibody (e.g. cross-competes) having the amino acid sequences set forth in SEQ ID NOs:4 and 10, SEQ ID NOs:1 and 5, SEQ ID NOs:1 and 6, SEQ ID NOs:2 and 7, SEQ ID NOs:3 and 8, or SEQ ID NOs:3 and 9, respectively.

The antibody-partner molecule conjugates of the present invention can be linked by a wide variety of linkers, such as those described throughout the application, as well as those know in the art. In one embodiment, the partner molecule is conjugated to the antibody by a chemical linker (i.e., a thiol linker, peptidyl linker, hydrazine linker or disulfide linker).

In yet another aspect, the present invention provides isolated nucleic acids encoding the antibodies (or antigen binding portions thereof) of the aforementioned antibody-partner molecule conjugates of the invention, as well as expression vectors and host cells.

As discussed throughout the application, antibody-partner molecule conjugates of the present invention can be used in a broad variety of diagnostic and therapeutic applications. For example, the antibody-partner molecule conjugates of the present invention can be administered to a subject in an amount effective to treat of prevent a disease (e.g., a cancer) characterized by growth of tumor cells expressing PTK7. Cancers that can be treated or prevented, include, but are not limited to colon cancer, lung cancer, breast cancer, pancreatic cancer, melanoma, acute myeloid leukemia, kidney cancer, bladder cancer, ovarian cancer and prostate cancer.

The invention also provides a bispecific molecule comprising an antibody, or antigen-binding portion thereof, of the invention, linked to a second functional moiety having a different binding specificity than said antibody, or antigen binding portion thereof.

Compositions comprising an antibody, or antigen-binding portion thereof, or immuno conjugate or bispecific molecule of the invention and a pharmaceutically acceptable carrier are also provided.

The present disclosure also provides isolated anti-PTK7 antibody-partner molecule conjugates that specifically bind to PTK7 with high affinity, particularly those comprising human monoclonal antibodies. Certain of such antibody-partner molecule conjugates are capable of being internalized into PTK7-expressing cells and are capable of mediating antigen dependent cellular cytotoxicity. This disclosure also provides methods for treating cancers, such as mesotheliomas, colon cancers, lung cancers, breast cancers, pancreatic cancers, melanomas, acute myeloid leukemias, kidney cancers, bladder cancers, ovarian cancers and prostate cancers, using an anti-PTK7 antibody-partner molecule conjugates disclosed herein.

Compositions comprising an antibody, or antigen-binding portion thereof, conjugated to a partner molecule of this disclosure are also provided. Partner molecules that can be advantageously conjugated to an antibody in an antibody partner molecule conjugate as disclosed herein include, but are not limited to, molecules as drugs, toxins, marker molecules (e.g., radioisotopes), proteins and therapeutic agents. Compositions comprising antibody-partner molecule conjugates and pharmaceutically acceptable carriers are also disclosed herein.

In one aspect, such antibody-partner molecule conjugates are conjugated via chemical linkers. In some embodiments, the linker is a peptidyl linker, and is depicted herein as (L⁴)ₚ-F-(L¹)ₘ. Other linkers include hydrazine and disulfide linkers, and is depicted herein as (L⁴)ₚ-H-(L¹)ₘ or (L⁴)ₚ-J-(L¹)ₘ, respectively. In addition to the linkers as being attached to the partner, the present invention also provides cleavable linker arms that are appropriate for attachment to essentially any molecular species.

Nucleic acid molecules encoding the antibodies, or antigen-binding portions thereof, of the invention are also encompassed by the invention, as well as expression vectors comprising such nucleic acids and host cells comprising such expression vectors. Moreover, the invention provides a transgenic mouse comprising human immunoglobulin heavy and light chain transgenes, wherein the mouse expresses an antibody of the invention, as well as hybridomas prepared from such a mouse, wherein the hybridoma produces the antibody of the invention.

In yet another aspect, the invention provides a method of treating or preventing a disease characterized by growth of tumor cells expressing PTK7, comprising administering to the subject the antibody, or antigen-binding portion thereof, of the invention in an amount effective to treat or prevent the disease. The disease can be, for example, cancer, e.g., colon cancer (including small intestine cancer), lung cancer, breast cancer, pancreatic cancer, melanoma (e.g., metastatic malignant melanoma), acute myeloid leukemia, kidney cancer, bladder cancer, ovarian cancer and prostate cancer.

In a preferred embodiment, the invention provides a method of treating cancer in vivo using an anti-PTK7 antibody. The anti-PTK7 antibody may be a murine, chimeric, humanized or human antibody. Examples of other cancers that may be treated using the methods of the invention include renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma and combinations of said cancers.

Other features and advantages of the instant invention will be apparent from the following detailed description and examples which should not be construed as limiting. The contents of all references, Genbank entries, patents and published patent applications cited throughout this application are expressly incorporated herein by reference.

### BRIEF DESCRIPTION OF THE DRAWINGS

FIG. 1A shows the nucleotide sequence (SEQ ID NO:41) and amino acid sequence (SEQ ID NO:1) of the heavy chain variable region of the 3G8 and 3G8a human monoclonal antibodies. The CDR1 (SEQ ID NO:11), CDR2 (SEQ ID NO:15) and CDR3 (SEQ ID NO:19) regions are delineated and the V, D and J germline derivations are indicated.

FIG. 1B shows the nucleotide sequence (SEQ ID NO:45) and amino acid sequence (SEQ ID NO:5) of the light chain variable region of the 3G8 human monoclonal antibody. The CDR1 (SEQ ID NO:23), CDR2 (SEQ ID NO:29) and CDR3 (SEQ ID NO:35) regions are delineated and the V and J germline derivations are indicated.

FIG. 1C shows the nucleotide sequence (SEQ ID NO:46) and amino acid sequence (SEQ ID NO:6) of the light chain variable region of the 3G8a human monoclonal antibody. The CDR1 (SEQ ID NO:24), CDR2 (SEQ ID NO:30) and CDR3 (SEQ ID NO:36) regions are delineated and the V and J germline derivations are indicated.

FIG. 2A shows the nucleotide sequence (SEQ ID NO:42) and amino acid sequence (SEQ ID NO:2) of the heavy chain variable region of the 4D5 human monoclonal antibody. The CDR1 (SEQ ID NO:12), CDR2 (SEQ ID NO:16) and CDR3 (SEQ ID NO:20) regions are delineated and the V, D and J germline derivations are indicated.

FIG. 2B shows the nucleotide sequence (SEQ ID NO:47) and amino acid sequence (SEQ ID NO:7) of the light chain variable region of the 4D5 human monoclonal antibody. The CDR1 (SEQ ID NO:25), CDR2 (SEQ ID NO:31) and CDR3 (SEQ ID NO:37) regions are delineated and the V and J germline derivations are indicated.

FIG. 3A shows the nucleotide sequence (SEQ ID NO:43) and amino acid sequence (SEQ ID NO:3) of the heavy chain variable region of the 12C6 human monoclonal antibodies. The CDR1 (SEQ ID NO:13), CDR2 (SEQ ID NO:17) and CDR3 (SEQ ID NO:21) regions are delineated and the V, D and J germline derivations are indicated.

FIG. 3B shows the nucleotide sequence (SEQ ID NO:48) and amino acid sequence (SEQ ID NO:8) of the light chain variable region of the 12C6 human monoclonal antibody. The CDR1 (SEQ ID NO:26), CDR2 (SEQ ID NO:32) and CDR3 (SEQ ID NO:38) regions are delineated and the V and J germline derivations are indicated.

FIG. 3C shows the nucleotide sequence (SEQ ID NO:49) and amino acid sequence (SEQ ID NO:9) of the light chain variable region of the 12C6a human monoclonal antibody. The CDR1 (SEQ ID NO:27), CDR2 (SEQ ID NO:33) and CDR3 (SEQ ID NO:39) regions are delineated and the V and J germline derivations are indicated.

FIG. 4A shows the nucleotide sequence (SEQ ID NO:44) and amino acid sequence (SEQ ID NO:4) of the heavy chain variable region of the 7C8 human monoclonal antibody. The CDR1 (SEQ ID NO:14), CDR2 (SEQ ID NO:18) and CDR3 (SEQ ID NO:22) regions are delineated and the V, D and J germline derivations are indicated.

FIG. 4B shows the nucleotide sequence (SEQ ID NO:50) and amino acid sequence (SEQ ID NO:10) of the light chain variable region of the 7C8 human monoclonal antibody. The CDR1 (SEQ ID NO:28), CDR2 (SEQ ID NO:34) and CDR3 (SEQ ID NO:40) regions are delineated and the V and J germline derivations are indicated.

FIG. 5 shows the alignment of the amino acid sequences of the heavy chain variable regions of 3G8 (SEQ ID NO: 1) and 3G8a (SEQ ID NO: 1) with the human germline V_{H} 3-30.3 amino acid sequence (SEQ ID NO:51) (JH4b germline disclosed as SEQ ID NO: 59).

FIG. 6 shows the alignment of the amino acid sequence of the heavy chain variable region of 4D5 (SEQ ID NO: 2) with the human germline V_{H} 3-30.3 amino acid sequence (SEQ ID NO:51) (JH4b germline disclosed as SEQ ID NO: 60).

FIG. 7 shows the alignment of the amino acid sequences of the heavy chain variable regions of 12C6 (SEQ ID NO: 3) and 12C6a (SEQ ID NO: 2) with the human germline V_{H} DP44 amino acid sequence (SEQ ID NO:52) (3-7, 3-23, and JH4b germlines disclosed as SEQ ID NOS 61-63, respectively).

FIG. 8 shows the alignment of the amino acid sequence of the heavy chain variable region of 7C8 (SEQ ID NO: 4) with the human germline V_{H} 3-33 amino acid sequence (SEQ ID NO:53) (JH6b germline disclosed as SEQ ID NO: 64).

FIG. 9 shows the alignment of the amino acid sequences of the light chain variable regions of 3G8 (SEQ ID NO: 5) and 3G8a (SEQ ID NO: 6) with the human germline Vₖ L15 amino acid sequence (SEQ ID NO:54) (JK1 germline disclosed as SEQ ID NO: 65).

FIG. 10 shows the alignment of the amino acid sequence of the light chain variable region of 4D5 (SEQ ID NO: 7) with the human germline Vₖ A10 amino acid sequence (SEQ ID NO:55) (JK5 germline disclosed as SEQ ID NO: 66).

FIG. 11 shows the alignment of the amino acid sequence of the light chain variable region of 12C6 (SEQ ID NO: 8) with the human germline Vₖ A27 amino acid sequence (SEQ ID NO:56) (JK2 germline disclosed as SEQ ID NO: 67).

FIG. 12 shows the alignment of the amino acid sequence of the light chain variable region of 12C6a (SEQ ID NO: 9) with the human germline Vₖ L15 amino acid sequence (SEQ ID NO:54) (JK2 germline disclosed as SEQ ID NO: 68).

FIG. 13 shows the alignment of the amino acid sequence of the light chain variable region of 7C8 (SEQ ID NO: 10) with the human germline Vₖ L6 amino acid sequence (SEQ ID NO:57) (JK3 germline disclosed as SEQ ID NO: 69).

FIG. 14 shows the results of flow cytometry experiments demonstrating that the human monoclonal antibody 7C8, directed against human PTK7, binds the cell surface of HEK3 cells transfected with full-length human PTK7.

FIG. 15 shows the results of ELISA experiments demonstrating that human monoclonal antibodies against human PTK7 specifically bind to PTK7.

FIG. 16 shows the results of flow cytometry experiments demonstrating that antibodies directed against human PTK7 binds the cell surface of Wilms' tumor cells.

FIG. 17 shows the results of flow cytometry experiments demonstrating that antibodies directed against human PTK7 binds the cell surface of a variety of cancer cell lines.

FIG. 18 shows the results of flow cytometry experiments demonstrating that antibodies directed against human PTK7 binds the cell surface of dendritic cells.

FIG. 19 shows the results of flow cytometry experiments demonstrating that antibodies directed against human PTK7 bind to CD4+ and CD8+ T-lymphocytes, but not to B-lymphocytes.

FIG. 20 shows the results of Hum-Zap internalization experiments demonstrating that human monoclonal antibodies against human PTK7 can internalize into PTK7+ cells. (A) Internalization of the human antibodies 3G8, 4D5 and 7C8 into Wilms' tumor cells. (B) Internalization of the human antibody 12C6 into Wilms' tumor cells. (C) Internalization of the human antibodies 7C8 and 12C6 into A-431 tumor cells. (D) Internalization of the human antibodies 7C8 and 12C6 into PC3 tumor cells.

FIG. 21 shows the results of a cell proliferation assay demonstrating that toxin-conjugated human monoclonal anti-PTK7 antibodies kill human kidney cancer cell lines.

FIG. 22 shows the results of a cell proliferation assay demonstrating that toxin-conjugated human monoclonal anti-PTK7 antibodies kill cell lines expressing low to high levels of PTK7.

FIG. 23 shows the results of an invasion assay demonstrating that anti-PTK7 antibodies inhibit the invasion mobility of cells expressing PTK7 on the cell surface.

FIG. 24 shows that anti-PTK7 antibodies conjugated to a toxin slowed pancreatic tumor progression in an in vivo xenograft model.

FIG. 25 shows that anti-PTK7 antibodies conjugated to a toxin slowed breast cancer progression in an in vivo xenograft model.

FIGS. 26A and 26B are graphs showing that epithelial-derived A431 and small cell lung-derived DMS79 tumors are reduced in in vivo models using 7C8-formula (m) conjugates. In FIG. 26A, median tumor volume was measured in mice treated with vehicle alone, an unmodified isotype-matched control antibody, an isotype-matched formula (m) conjugate, and 7C8-formula (m). FIG. 26B shows that treatment with 7C8-formula (m) caused complete regression of DMS79 small cell lung carcinoma tumors in SCID mice.

FIG. 27 is a graph showing that the 7C8-formula (m) conjugate does not cause significant weight loss in the in vivo SCID xenograft mouse model.

FIG. 28 is a diagram of the chemical structure of formula (m).

### DETAILED DESCRIPTION OF THE INVENTION

In one aspect, the present invention relates to isolated monoclonal antibodies, particularly human monoclonal antibodies, that bind specifically to PTK7. In certain embodiments, the antibodies of the invention exhibit one or more desirable functional properties, such as high affinity binding to PTK7 and/or the ability to inhibit growth of tumor cells in vitro or in vivo. In certain embodiments, the antibodies of the invention are derived from particular heavy and light chain germline sequences and/or comprise particular structural features such as CDR regions comprising particular amino acid sequences. The invention provides isolated antibodies, methods of making such antibodies, immunoconjugates and bispecific molecules comprising such antibodies and pharmaceutical compositions containing the antibodies, immunoconjugates or bispecific molecules of the invention. The invention also relates to methods of using the antibodies, such as to treat diseases such as cancer.

In order that the present invention may be more readily understood, certain terms are first defined. Additional definitions are set forth throughout the detailed description.

The terms “PTK7” and “CCK4” are used interchangeably and include variants, isoforms and species homologs of human PTK7. Accordingly, human antibodies of the invention may, in certain cases, cross-react with PTK7 from species other than human. In certain embodiments, the antibodies may be completely specific for one or more human PTK7 and may not exhibit species or other types of non-human cross-reactivity. The complete amino acid sequence of an exemplary human PTK7 has Genbank accession number NM_{—}002821 (SEQ ID NO:58).

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between a variety of signal transduction molecules that play a role in the transmission of a signal from one portion of a cell to another portion of a cell. As used herein, the phrase “cell surface receptor” includes, for example, molecules and complexes of molecules capable of receiving a signal and the transmission of such a signal across the plasma membrane of a cell. An example of a “cell surface receptor” of the present invention is the PTK7 receptor.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., PTK7). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3.sup.rd ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds PTK7 is substantially free of antibodies that specifically bind antigens other than PTK7). An isolated antibody that specifically binds PTK7 may, however, have cross-reactivity to other antigens, such as PTK7 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” is intended to refer to molecules capable of mimicking an antibody's ability to bind an antigen, but which are not limited to native antibody structures. Examples of such antibody mimetics include, but are not limited to, Affibodies, DARPins, Anticalins, Avimers, and Versabodies, all of which employ binding structures that, while they mimic traditional antibody binding, are generated from and function via distinct mechanisms.

As used herein, the term “partner molecule” refers to the entity which is conjugated to an antibody in an antibody partner molecule conjugate. Examples of partner molecules include drugs, toxins, marker molecules (including, but not limited to peptide and small molecule markers, such as fluorochrome markers, as well as single atom markers, such as radioisotopes), proteins and therapeutic agents.

As used herein, an antibody that “specifically binds to human PTK7” is intended to refer to an antibody that binds to human PTK7 with a K_{D} of 1×10⁻⁷ M or less, more preferably 5×10⁻⁸ M or less, more preferably 1×10⁻⁸M or less, more preferably 5×10⁻⁹ M or less.

The term “does not substantially bind” to a protein or cells, as used herein, means does not bind or does not bind with a high affinity to the protein or cells, i.e. binds to the protein or cells with a K_{D} of 1×10⁻⁶ M or more, more preferably 1×10⁻⁵M or more, more preferably 1×10⁻⁴ M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity” for an IgG antibody refers to an antibody having a K_{D} of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The symbol “-”, whether utilized as a bond or displayed perpendicular to a bond, indicates the point at which the displayed moiety is attached to the remainder of the molecule, solid support, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, and S, and wherein the nitrogen, carbon and sulfur atoms may optionally be oxidized and the nitrogen heteroatom may optionally be quaternized. The heteroatom(s) O, N, S, and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino,” “alkylsulfonyl,” and “alkylthio” (or thioalkoxy) are used in their conventional sense, and refer to those alkyl groups attached to the remainder of the molecule via an oxygen atom, an amino group, an SO₂ group or a sulfur atom, respectively. The term “arylsulfonyl” refers to an aryl group attached to the remainder of the molecule via an SO₂ group, and the term “sulfhydryl” refers to an SH group.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5, 6, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R′″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “diphosphate” includes but is not limited to an ester of phosphoric acid containing two phosphate groups. The term “triphosphate” includes but is not limited to an ester of phosphoric acid containing three phosphate groups. For example, particular drugs having a diphosphate or a triphosphate include:

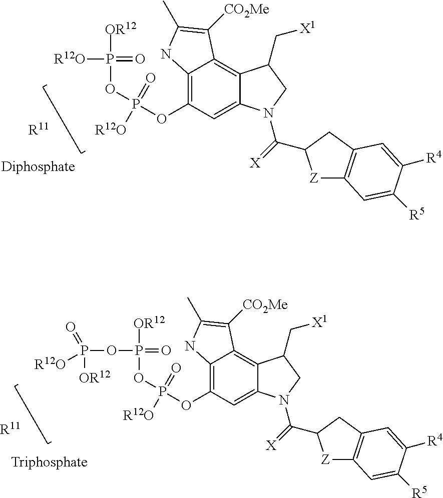

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

Various aspects of the invention are described in further detail in the following subsections.

### Anti-PTK7 Antibodies

The antibodies of the invention are characterized by particular functional features or properties of the antibodies. For example, the antibodies bind specifically to PTK7. Preferably, an antibody of the invention binds to PTK7 with high affinity, for example with a K_{D} of 1×10⁻⁷M or less. The anti-PTK7 antibodies of the invention preferably exhibit one or more of the following characteristics:

(a) specifically binds to human PTK7; or

(b) binds to a Wilms' tumor cell line (ATCC Acc No. CRL-1441).

Preferrably, the antibody binds to human PTK7 with a K_{D} of 5×10⁻⁸ M or less, binds to human PTK7 with a K_{D} of 1×10⁻⁸ M or less, binds to human PTK7 with a K_{D} of 5×10⁻⁹ M or less, or binds to human PTK7 with a K_{D} of between 1×10⁻⁸M and 1×10⁻¹⁰ M or less. Preferrably, the antibody binds to Wilms' tumor cells with an EC₅₀ of 4.0 nM or less, or binds to Wilms' tumor cells with an EC₅₀ of 3.5 nM or less. Standard assays to evaluate the binding ability of the antibodies toward PTK7 are known in the art, including for example, ELISAs, Western blots and RIAs. The binding kinetics (e.g., binding affinity) of the antibodies also can be assessed by standard assays known in the art, such as by ELISA, Scatchard and Biacore analysis. As another example, the antibodies of the present invention may bind to a kidney carcinoma tumor cell line, for example, the Wilms' tumor cell line. Suitable assays for evaluating any of the above-described characteristics are described in detail in the Examples.

### Monoclonal Antibodies 3G8, 3G8a, 4D5, 12C6, 12C6a and 7C8

Preferred antibodies of the invention are the human monoclonal antibodies 3G8, 3G8a, 4D5, 12C6, 12C6a and 7C8, isolated and structurally characterized as described in Examples 1 and 2. Those having ordinary skill in the art shall appreciate that the antibodies 3G8 and 3G8a, as well as the antibodies 12C6 and 12C6a have the same heavy chain sequence, while differing in their light chain sequences. The V_{H} amino acid sequences of 3G8, 3G8a, 4D5, 12C6, 12C6a and 7C8 are shown in SEQ ID NOs: 1 (3G8 and 3G8a), 2 (4D5), 3 (12C6 and 12C6a) and 4 (7C8). The V_{L} amino acid sequences of 3G8, 3G8a, 4D5, 12C6, 12C6a, and 7C8 are shown in SEQ ID NOs: 5, 6, 7, 8, 9 and 10, respectively.

Given that each of these antibodies can bind to PTK7, the V_{H} and V_{L} sequences can be “mixed and matched” to create other anti-PTK7 binding molecules of the invention. PTK7 binding of such “mixed and matched” antibodies can be tested using the binding assays described above and in the Examples (e.g., ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:

(a) a heavy chain variable region comprising an amino acid sequence selected from the group consisting of SEQ ID NOs: 1, 2, 3 and 4; and

(b) a light chain variable region comprising an amino acid sequence selected from the group consisting of SEQ ID NOs: 5, 6, 7, 8, 9 and 10;

wherein the antibody specifically binds PTK7, preferably human PTK7.

Preferred heavy and light chain combinations include:

(a) a heavy chain variable region comprising the amino acid sequence of SEQ ID NO:1; and (b) a light chain variable region comprising the amino acid sequence of SEQ ID NO:5; or

(a) a heavy chain variable region comprising the amino acid sequence of SEQ ID NO:1; and (b) a light chain variable region comprising the amino acid sequence of SEQ ID NO:6; or

(a) a heavy chain variable region comprising the amino acid sequence of SEQ ID NO:2; and (b) a light chain variable region comprising the amino acid sequence of SEQ ID NO:7; or

(a) a heavy chain variable region comprising the amino acid sequence of SEQ ID NO:3; and (b) a light chain variable region comprising the amino acid sequence of SEQ ID NO:8; or

(a) a heavy chain variable region comprising the amino acid sequence of SEQ ID NO:3; and (b) a light chain variable region comprising the amino acid sequence of SEQ ID NO:9; or

(a) a heavy chain variable region comprising the amino acid sequence of SEQ ID NO:4; and (b) a light chain variable region comprising the amino acid sequence of SEQ ID NO:10.

In another aspect, the invention provides antibodies that comprise the heavy chain and light chain CDR1s, CDR2s and CDR3s of 3G8, 3G8a, 4D5, 12C6, 12C6a and 7C8, or combinations thereof. The amino acid sequences of the V_{H} CDR1s of 3G8, 3G8a, 4D5, 12C6, 12C6a and 7C8 are shown in SEQ ID NOs: 11 (3G8 and 3G8a), 12 (4D5), 13 (12C6 and 12C6a) and 14 (7C8). The amino acid sequences of the V_{H} CDR2s of 3G8, 3G8a, 4D5, 12C6, 12C6a and 7C8 are shown in SEQ ID NOs: 15 (3G8 and 3G8a), 16 (4D5), 17 (12C6 and 12C6a) and 18 (7C8). The amino acid sequences of the V_{H} CDR3s of 3G8, 3G8a, 4D5, 12C6, 12C6a and 7C8 are shown in SEQ ID NOs: 19 (3G8 and 3G8a), 20 (4D5), 21 (12C6 and 12C6a) and 22 (7C8). The amino acid sequences of the Vₖ CDR1s of 3G8, 3G8a, 4D5, 12C6, 12C6a and 7C8 are shown in SEQ ID NOs: 23, 24, 25, 26, 27 and 28, respectively. The amino acid sequences of the Vₖ CDR2s of 3G8, 3G8a, 4D5, 12C6, 12C6a and 7C8 are shown in SEQ ID NOs: 29, 30, 31, 32, 33 and 34, respectively. The amino acid sequences of the Vₖ CDR3s of 3G8, 3G8a, 4D5, 12C6, 12C6a and 7C8 are shown in SEQ ID NOs: 35, 36, 37, 38, 39 and 40, respectively. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to PTK7 and that antigen-binding specificity is provided primarily by the CDR1, CDR2, and CDR3 regions, the V_{H} CDR1, CDR2, and CDR3 sequences and Vₖ CDR1, CDR2, and CDR3 sequences can be “mixed and matched” (i.e., CDRs from different antibodies can be mixed and match, although each antibody must contain a V_{H} CDR1, CDR2, and CDR3 and a Vₖ CDR1, CDR2, and CDR3) to create other anti-PTK7 binding molecules of the invention. PTK7 binding of such “mixed and matched” antibodies can be tested using the binding assays described above and in the Examples (e.g., ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies antibodies 3G8, 3G8a, 4D5, 12C6, 12C6a and 7C8.

Accordingly, in another aspect, the invention provides an isolated monoclonal antibody, or antigen binding portion thereof comprising:

(a) a heavy chain variable region CDR1 comprising an amino acid sequence selected from the group consisting of SEQ ID NOs: 11, 12, 13 and 14;

(b) a heavy chain variable region CDR2 comprising an amino acid sequence selected from the group consisting of SEQ ID NOs: 15, 16, 17 and 18;

(c) a heavy chain variable region CDR3 comprising an amino acid sequence selected from the group consisting of SEQ ID NOs: 19, 20, 21 and 22;

(d) a light chain variable region CDR1 comprising an amino acid sequence selected from the group consisting of SEQ ID NOs: 23, 24, 25, 26, 27 and 28;

(e) a light chain variable region CDR2 comprising an amino acid sequence selected from the group consisting of SEQ ID NOs: 29, 30, 31, 32, 33 and 34; and

(f) a light chain variable region CDR3 comprising an amino acid sequence selected from the group consisting of SEQ ID NOs: 35, 36, 37, 38, 39 and 40;

wherein the antibody specifically binds PTK7, preferably human PTK7.

In a preferred embodiment, the antibody comprises:

(a) a heavy chain variable region CDR1 comprising SEQ ID NO:11;

(b) a heavy chain variable region CDR2 comprising SEQ ID NO:15;

(c) a heavy chain variable region CDR3 comprising SEQ ID NO:19;

(d) a light chain variable region CDR1 comprising SEQ ID NO:23;

(e) a light chain variable region CDR2 comprising SEQ ID NO:29; and

(f) a light chain variable region CDR3 comprising SEQ ID NO:35.

In another preferred embodiment, the antibody comprises:

(a) a heavy chain variable region CDR1 comprising SEQ ID NO:11;

(b) a heavy chain variable region CDR2 comprising SEQ ID NO:15;

(c) a heavy chain variable region CDR3 comprising SEQ ID NO:19;

(d) a light chain variable region CDR1 comprising SEQ ID NO:24;

(e) a light chain variable region CDR2 comprising SEQ ID NO:30; and

(f) a light chain variable region CDR3 comprising SEQ ID NO:36.

In another preferred embodiment, the antibody comprises:

(a) a heavy chain variable region CDR1 comprising SEQ ID NO:12;

(b) a heavy chain variable region CDR2 comprising SEQ ID NO:16;

(c) a heavy chain variable region CDR3 comprising SEQ ID NO:20;

(d) a light chain variable region CDR1 comprising SEQ ID NO:25;

(e) a light chain variable region CDR2 comprising SEQ ID NO:31; and

(f) a light chain variable region CDR3 comprising SEQ ID NO:37.

In another preferred embodiment, the antibody comprises:

(a) a heavy chain variable region CDR1 comprising SEQ ID NO:13;

(b) a heavy chain variable region CDR2 comprising SEQ ID NO:17;

(c) a heavy chain variable region CDR3 comprising SEQ ID NO:21;

(d) a light chain variable region CDR1 comprising SEQ ID NO:26;

(e) a light chain variable region CDR2 comprising SEQ ID NO:32; and

(f) a light chain variable region CDR3 comprising SEQ ID NO:38.

In another preferred embodiment, the antibody comprises:

(a) a heavy chain variable region CDR1 comprising SEQ ID NO:13;

(b) a heavy chain variable region CDR2 comprising SEQ ID NO:17;

(c) a heavy chain variable region CDR3 comprising SEQ ID NO:21;

(d) a light chain variable region CDR1 comprising SEQ ID NO:27;

(e) a light chain variable region CDR2 comprising SEQ ID NO:33; and

(f) a light chain variable region CDR3 comprising SEQ ID NO:39.

In another preferred embodiment, the antibody comprises:

(a) a heavy chain variable region CDR1 comprising SEQ ID NO:14;

(b) a heavy chain variable region CDR2 comprising SEQ ID NO:18;

(c) a heavy chain variable region CDR3 comprising SEQ ID NO:22;

(d) a light chain variable region CDR1 comprising SEQ ID NO:28;

(e) a light chain variable region CDR2 comprising SEQ ID NO:34; and

(f) a light chain variable region CDR3 comprising SEQ ID NO:40.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin αᵥβ₃ antibodies using a heavy and light chain variable CDR3 domain of a murine anti-integrin αᵥβ₃ antibody LM609 wherein each member antibody comprises a distinct sequence outside the CDR3 domain and capable of binding the same epitope as the parent murine antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad. Sci. U.S.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); and Ditzel et al., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab); Berezov et al., BIAjournal 8:Scientific Review 8 (2001) (describing peptide mimetics based on the CDR3 of an anti-HER2 monoclonal antibody; Igarashi et al., J. Biochem (Tokyo) 117:452-7 (1995) (describing a 12 amino acid synthetic polypeptide corresponding to the CDR3 domain of an anti-phosphatidylserine antibody); Bourgeois et al., J. Virol 72:807-10 (1998) (showing that a single peptide derived form the heavy chain CDR3 domain of an anti-respiratory syncytial virus (RSV) antibody was capable of neutralizing the virus in vitro); Levi et al., Proc. Natl. Acad. Sci. U.S.A. 90:4374-8 (1993) (describing a peptide based on the heavy chain CDR3 domain of a murine anti-HIV antibody); Polymenis and Stoller, J. Immunol. 152:5218-5329 (1994) (describing enabling binding of an scFv by grafting the heavy chain CDR3 region of a Z-DNA-binding antibody) and Xu and Davis, Immunity 13:37-45 (2000) (describing that diversity at the heavy chain CDR3 is sufficient to permit otherwise identical IgM molecules to distinguish between a variety of hapten and protein antigens). See also, U.S. Pat. Nos. 6,951,646; 6,914,128; 6,090,382; 6,818,216; 6,156,313; 6,827,925; 5,833,943; 5,762,905 and 5,760,185, describing patented antibodies defined by a single CDR domain. Each of these references is hereby incorporated by reference in its entirety.

Accordingly, the present invention provides monoclonal antibodies comprising one or more heavy and/or light chain CDR3 domains from an antibody derived from a human or non-human animal, wherein the monoclonal antibody is capable of specifically binding to PTK7. Within certain aspects, the present invention provides monoclonal antibodies comprising one or more heavy and/or light chain CDR3 domain from a non-human antibody, such as a mouse or rat antibody, wherein the monoclonal antibody is capable of specifically binding to PTK7. Within some embodiments, such inventive antibodies comprising one or more heavy and/or light chain CDR3 domain from a non-human antibody (a) are capable of competing for binding with; (b) retain the functional characteristics; (c) bind to the same epitope; and/or (d) have a similar binding affinity as the corresponding parental non-human antibody.

Within other aspects, the present invention provides monoclonal antibodies comprising one or more heavy and/or light chain CDR3 domain from a human antibody, such as, for example, a human antibody obtained from a non-human animal, wherein the human antibody is capable of specifically binding to PTK7. Within other aspects, the present invention provides monoclonal antibodies comprising one or more heavy and/or light chain CDR3 domain from a first human antibody, such as, for example, a human antibody obtained from a non-human animal, wherein the first human antibody is capable of specifically binding to PTK7 and wherein the CDR3 domain from the first human antibody replaces a CDR3 domain in a human antibody that is lacking binding specificity for PTK7 to generate a second human antibody that is capable of specifically binding to PTK7. Within some embodiments, such inventive antibodies comprising one or more heavy and/or light chain CDR3 domain from the first human antibody (a) are capable of competing for binding with; (b) retain the functional characteristics; (c) bind to the same epitope; and/or (d) have a similar binding affinity as the corresponding parental first human antibody. In preferred embodiments, the first human antibody is 3G8, #g8a, 4D5, 12C6, 12C6a or 7C8.

### Antibodies Having Particular Germline Sequences

In certain embodiments, an antibody of the invention comprises a heavy chain variable region from a particular germline heavy chain immunoglobulin gene and/or a light chain variable region from a particular germline light chain immunoglobulin gene.

For example, in a preferred embodiment, the invention provides an isolated monoclonal antibody, or an antigen-binding portion thereof, comprising a heavy chain variable region that is the product of or derived from a human V_{H} 3-30.3 gene, wherein the antibody specifically binds PTK7, preferably human PTK7. In another preferred embodiment, the invention provides an isolated monoclonal antibody, or an antigen-binding portion thereof, comprising a heavy chain variable region that is the product of or derived from a human V_{H} DP44 gene, wherein the antibody specifically binds PTK7, preferably human PTK7. In another preferred embodiment, the invention provides an isolated monoclonal antibody, or an antigen-binding portion thereof, comprising a heavy chain variable region that is the product of or derived from a human V_{H} 3-33 gene, wherein the antibody specifically binds PTK7, preferably human PTK7. In yet another preferred embodiment, the invention provides an isolated monoclonal antibody, or an antigen-binding portion thereof, comprising a light chain variable region that is the product of or derived from a human V_{K} L15 gene, wherein the antibody specifically binds PTK7, preferably human PTK7. In yet another preferred embodiment, the invention provides an isolated monoclonal antibody, or an antigen-binding portion thereof, comprising a light chain variable region that is the product of or derived from a human V_{K} A10 gene, wherein the antibody specifically binds PTK7, preferably human PTK7. In yet another preferred embodiment, the invention provides an isolated monoclonal antibody, or an antigen-binding portion thereof, comprising a light chain variable region that is the product of or derived from a human V_{K} A27 gene, wherein the antibody specifically binds PTK7, preferably human PTK7. In yet another preferred embodiment, the invention provides an isolated monoclonal antibody, or an antigen-binding portion thereof, comprising a light chain variable region that is the product of or derived from a human V_{K} L6 gene, wherein the antibody specifically binds PTK7, preferably human PTK7. In yet another preferred embodiment, the invention provides an isolated monoclonal antibody, or antigen-binding portion thereof, wherein the antibody:

(a) comprises a heavy chain variable region that is the product of or derived from a human V_{H} 3-30.3, DP44 or 3-33 gene (which gene encodes the amino acid sequence set forth in SEQ ID NOs: 51, 52 or 53, respectively);

(b) comprises a light chain variable region that is the product of or derived from a human V_{K} L15, A10, A27 or L6 gene (which gene encodes the amino acid sequence set forth in SEQ ID NO:54, 55, 56 or 57, respectively); and

(c) specifically binds to PTK7.

Examples of antibodies having V_{H} and V_{K} of V_{H} 3-30.3 and V_{K} L15, respectively, are 3G8 and 3G8a. An example of an antibody having V_{H} and V_{K} of V_{H} 3-30.3 and V_{K} A10, respectively is 4D5. An example of an antibody having V_{H} and V_{K} of V_{H} DP44 and V_{K} A27, respectively is 12C6. An example of an antibody having V_{H} and V_{K} of V_{H} DP44 and V_{K} L15, respectively is 12C6a. An example of an antibody having V_{H} and V_{K} of V_{H} 3-33 and V_{K} L6, respectively is 7C8.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-PTK7 antibodies of the invention.

For example, the invention provides an isolated monoclonal antibody, or antigen binding portion thereof, comprising a heavy chain variable region and a light chain variable region, wherein:
(a) the heavy chain variable region comprises an amino acid sequence that is at least 80% homologous to an amino acid sequence selected from the group consisting of SEQ ID NOs: 1, 2, 3 and 4;
(b) the light chain variable region comprises an amino acid sequence that is at least 80% homologous to an amino acid sequence selected from the group consisting of SEQ ID NOs: 5, 6, 7, 8, 9 and 10; and
the antibody exhibits one or more of the following properties:
(c) the antibody binds to human PTK7 with a K_{D} of 1×10⁻⁷ M or less;
(d) the antibody binds to the Wilms' tumor cell line.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 41, 42, 43, 44, 45, 46, 47, 48, 49 and 50, followed by testing of the encoded altered antibody for retained function (i.e., the functions set forth in (c) and (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 3G8, 3G8a, 4D5, 12C6, 12C6a or 7C8), or conservative modifications thereof, and wherein the antibodies retain the desired functional properties of the anti-PTK7 antibodies of the invention. It is understood in the art that certain conservative sequence modification can be made which do not remove antigen binding. See, for example, Brummell et al. (1993) Biochem 32:1180-8 (describing mutational analysis in the CDR3 heavy chain domain of antibodies specific for Salmonella); de Wildt et al. (1997) Prot. Eng. 10:835-41 (describing mutation studies in anti-UA1 antibodies); Komissarov et al. (1997) J. Biol. Chem. 272:26864-26870 (showing that mutations in the middle of HCDR3 led to either abolished or diminished affinity); Hall et al. (1992) J. Immunol. 149:1605-12 (describing that a single amino acid change in the CDR3 region abolished binding activity); Kelley and O'Connell (1993) Biochem. 32:6862-35 (describing the contribution of Tyr residues in antigen binding); Adib-Conquy et al. (1998) Int. Immunol. 10:341-6 (describing the effect of hydrophobicity in binding) and Beers et al. (2000) Clin. Can. Res. 6:2835-43 (describing HCDR3 amino acid mutants). Accordingly, the invention provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:

(a) the heavy chain variable region CDR3 sequence comprises an amino acid sequence selected from the group consisting of amino acid sequences of SEQ ID NOs: 19, 20, 21 and 22, and conservative modifications thereof;

(b) the light chain variable region CDR3 sequence comprises an amino acid sequence selected from the group consisting of amino acid sequence of SEQ ID NOs: 35, 36, 37, 38, 39 and 40, and conservative modifications thereof; and
the antibody exhibits one or more of the following properties:

(c) specifically binds to human PTK7; and

(d) binds to a Wilms' tumor cell line (ATCC Acc No. CRL-1441).

In a preferred embodiment, the heavy chain variable region CDR2 sequence comprises an amino acid sequence selected from the group consisting of amino acid sequences of SEQ ID NOs: 15, 16, 17 and 18, and conservative modifications thereof; and the light chain variable region CDR2 sequence comprises an amino acid sequence selected from the group consisting of amino acid sequences of SEQ ID NOs: 29, 30, 31, 32, 33 and 34, and conservative modifications thereof. In another preferred embodiment, the heavy chain variable region CDR1 sequence comprises an amino acid sequence selected from the group consisting of amino acid sequences of SEQ ID NOs: 11, 12, 13 and 14, and conservative modifications thereof; and the light chain variable region CDR1 sequence comprises an amino acid sequence selected from the group consisting of amino acid sequences of SEQ ID NOs: 23, 24, 25, 26, 27 and 28, and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) and (d) above) using the functional assays described herein.

Antibodies that Bind to the Same Epitope as Anti-PTK7 Antibodies of the Invention

In another embodiment, the invention provides antibodies that bind an epitope on human PTK7 recognized by any of the PTK7 monoclonal antibodies of the invention (i.e., antibodies that have the ability to cross-compete for binding to PTK7 with any of the monoclonal antibodies of the invention). In preferred embodiments, the reference antibody for cross-competition studies can be the monoclonal antibody 3G8 (having V_{H} and V_{L} sequences as shown in SEQ ID NOs: 1 and 5, respectively), or the monoclonal antibody 3G8a (having V_{H} and V_{L} sequences as shown in SEQ ID NOs: 1 and 6, respectively), or the monoclonal antibody 4D5 (having V_{H} and V_{L} sequences as shown in SEQ ID NOs: 2 and 7, respectively), or the monoclonal antibody 12C6 (having V_{H} and V_{L} sequences as shown in SEQ ID NOs: 3 and 8, respectively), or the monoclonal antibody 12C6a (having V_{H} and V_{L} sequences as shown in SEQ ID NOs: 3 and 9, respectively), or the monoclonal antibody 7C8 (having V_{H} and V_{L} sequences as shown in SEQ ID NOs: 4 and 10, respectively).

Such cross-competing antibodies can be identified based on their ability to cross-compete with 3G8, 3G8a, 4D5, 12C6, 12C6a or 7C8 in standard PTK7 binding assays. For example, BIAcore analysis, ELISA assays or flow cytometry may be used to demonstrate cross-competition with the antibodies of the current invention. The ability of a test antibody to inhibit the binding of, for example, 3G8, 3G8a, 4D5, 12C6, 12C6a or 7C8, to human PTK7 demonstrates that the test antibody can compete with 3G8, 3G8a, 4D5, 12C6, 12C6a or 7C8 for binding to human PTK7 and thus binds to the same epitope on human PTK7 as 3G8, 3G8a, 4D5, 12C6, 12C6a or 7C8. In a preferred embodiment, the antibody that binds to the same epitope on human PTK7 is recognized by 3G8 (having V_{H} and V_{L} sequences as shown in SEQ ID NOs:1 and 5, respectively) 3G8a (having V_{H} and V_{L} sequences as shown in SEQ ID NOs:1 and 6, respectively), 4D5 (having V_{H} and V_{L} sequences as shown in SEQ ID NOs:2 and 7, respectively), 12C6 (having V_{H} and V_{L} sequences as shown in SEQ ID NOs:3 and 8, respectively), 12C6a (having V_{H} and V_{L} sequences as shown in SEQ ID NOs:3 and 9, respectively) or 7C8 (having V_{H} and V_{L} sequences as shown in SEQ ID NOs:4 and 10, respectively). In a preferred embodiment the antibody that binds to the same epitope on human PTK7 as is recognized by 3G8, 3G8a, 4D5, 12C6, 12C6a or 7C8 is a human monoclonal antibody. Such human monoclonal antibodies can be prepared and isolated as described in the Examples.

### Engineered and Modified Antibodies

An antibody of the invention further can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated monoclonal antibody, or antigen binding portion thereof, comprising a heavy chain variable region comprising CDR1, CDR2, and CDR3 sequences comprising an amino acid sequence selected from the group consisting of SEQ ID NOs: 11, 12, 13 and 14, SEQ ID NOs: 15, 16, 17 and 18 and SEQ ID NOs: 19, 20, 21 and 22, respectively, and a light chain variable region comprising CDR1, CDR2, and CDR3 sequences comprising an amino acid sequence selected from the group consisting of SEQ ID NOs: 23, 24, 25, 26, 27 and 28, SEQ ID NOs: 29, 30, 31, 32, 33 and 34 and SEQ ID NOs: 35, 36, 37, 38, 39 and 40, respectively. Thus, such antibodies contain the V_{H} and V_{L} CDR sequences of monoclonal antibodies 3G8, 3G8a, 4D5, 12C6, 12C6a or 7C8 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and 3-23 (AJ406678). Other human germline sequence databases, such as that available from IMGT (http://imgt.cines.fr), can be searched similarly to VBASE as described above.

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Preferred framework sequences for use in the antibodies of the invention are those that are structurally similar to the framework sequences used by selected antibodies of the invention, e.g., similar to the V_{H} 3-30.3 framework sequences (SEQ ID NO:51) and/or the V_{H} DP44 framework sequences (SEQ ID NO:52) and/or the V_{H} 3-33 framework sequences (SEQ ID NO:53) and/or the V_{K} L15 framework sequences (SEQ ID NO:54) and/or the V_{K} A10 framework sequences (SEQ ID NO:55) and/or the V_{K} L15 framework sequences (SEQ ID NO:54) and/or the V_{K} A27 framework sequences (SEQ ID NO:56) and/or the V_{K} L15 framework sequences (SEQ ID NO:54) and/or the V_{K} L6 framework sequences (SEQ ID NO:57) used by preferred monoclonal antibodies of the invention. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the invention provides isolated anti-PTK7 monoclonal antibodies, or antigen binding portions thereof, comprising a heavy chain variable region comprising: (a) a V_{H} CDR1 region comprising an amino acid sequence selected from the group consisting of SEQ ID NOs: 11, 12, 13 and 14, or an amino acid sequence having one, two, three, four or five amino acid substitutions, deletions or additions as compared to SEQ ID NOs: 11, 12, 13 and 14; (b) a V_{H} CDR2 region comprising an amino acid sequence selected from the group consisting of SEQ ID NOs: 15, 16, 17 and 18, or an amino acid sequence having one, two, three, four or five amino acid substitutions, deletions or additions as compared to SEQ ID NOs: 15, 16, 17 and 18; (c) a V_{H} CDR3 region comprising an amino acid sequence selected from the group consisting of SEQ ID NOs: 19, 20, 21 and 22, or an amino acid sequence having one, two, three, four or five amino acid substitutions, deletions or additions as compared to SEQ ID NOs: 19, 20, 21 and 22; (d) a V_{K} CDR1 region comprising an amino acid sequence selected from the group consisting of SEQ ID NOs: 23, 24, 25, 26, 27 and 28, or an amino acid sequence having one, two, three, four or five amino acid substitutions, deletions or additions as compared to SEQ ID NOs: 23, 24, 25, 26, 27 and 28; (e) a V_{K} CDR2 region comprising an amino acid sequence selected from the group consisting of SEQ ID NOs: 29, 30, 31, 32, 33 and 34, or an amino acid sequence having one, two, three, four or five amino acid substitutions, deletions or additions as compared to SEQ ID NOs: 29, 30, 31, 32, 33 and 34; and (f) a V_{K} CDR3 region comprising an amino acid sequence selected from the group consisting of SEQ ID NOs: 35, 36, 37, 38, 39 and 40, or an amino acid sequence having one, two, three, four or five amino acid substitutions, deletions or additions as compared to SEQ ID NOs: 35, 36, 37, 38, 39 and 40.

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

For example, for 3G8 (and 3G8a), amino acid residue #28 (within FR1) of V_{H} is an isoleucine whereas this residue in the corresponding V_{H} 3-30.3 germline sequence is a threonine. To return the framework region sequences to their germline configuration, the somatic mutations can be “backmutated” to the germline sequence by, for example, site-directed mutagenesis or PCR-mediated mutagenesis (e.g., residue #28 of FR1 of the V_{H} of 3G8 (and 3G8a) can be “backmutated” from isoleucine to threonine).

As another example, for 12C6 (and 12C6a), amino acid residue #44 (within FR2) of V_{H} is a threonine whereas this residue in the corresponding V_{H} DP44 germline sequence is a glycine. To return the framework region sequences to their germline configuration, for example, residue #44 (residue #9 of FR2) of the V_{H} of 12C6 (and 12C6a) can be “backmutated” from threonine to glycine. Such “backmutated” antibodies are also intended to be encompassed by the invention.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Can et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fe region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al, and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al., Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Antibody Fragments and Antibody Mimetics

The inventions disclosed herein are not limited traditional antibodies as the antigen binding component and may be practiced through the use of antibody fragments and antibody mimetics. A wide variety of antibody fragment and antibody mimetic technologies have now been developed and are widely known in the art.

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

Nanobodies are antibody-derived proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential.

Nanobodies combine the advantages of conventional antibodies with important features of small molecule drugs. Like conventional antibodies, Nanobodies show high target specificity and affinity and low inherent toxicity. Furthermore, Nanobodies are extremely stable, can be administered by means other than injection (see, e.g., WO 2004/041867) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety).

The Nanoclone method (see, e.g., WO 06/079372, which is herein incorporated by reference in its entirety) generates Nanobodies against a desired target, based on automated high-throughout selection of B-cells and could be used in the context of the instant invention.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

Affibody molecules are affinity proteins based on a 58-amino acid residue protein domain derived from a three helix bundle IgG-binding domain of staphylococcal protein A. This domain has been used as a scaffold for the construction of combinatorial phagemid libraries, from which Affibody variants targeting the desired molecules can be selected using phage display technology (Nord et al., Nat Biotechnol 1997; 15:772-7; Ronmark et al., Eur J Biochem 2002; 269:2647-55). The simple, robust structure and low molecular weight (6 kDa) of Affibody molecules makes them suitable for a wide variety of applications, such as detection reagents and inhibitors of receptor interactions. Further details on Affibodies are found in U.S. Pat. No. 5,831,012 which is incorporated by reference in its entirety. Labelled Affibodies may also be useful in imaging applications for determining abundance of isoforms.

DARPins (Designed Ankyrin Repeat Proteins) embody DRP (Designed Repeat Protein) antibody mimetic technology that exploits the binding abilities of non-antibody polypeptides. Repeat proteins, such as ankyrin and leucine-rich repeat proteins, are ubiquitous binding molecules that, unlike antibodies, occur intra- and extracellularly. Their unique modular architecture features repeating structural units (repeats) that stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains Additional information regarding DARPins and other DRP technologies can be found in US 2004/0132028 and WO 02/20565, both of which are incorporated by reference.

Anticalins are another antibody mimetic technology. In this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved B-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids, which is marginally larger than a single immunoglobulin domain.

Lipocalins can be cloned and their loops subjected to engineering to create Anticalins. Libraries of structurally diverse Anticalins have been generated and Anticalin display allows the selection and screening of binding function, followed by the expression and production of soluble protein for further analysis in prokaryotic or eukaryotic systems. Studies have demonstrated that Anticalins can be developed that are specific for virtually any human target protein and binding affinities in the nanomolar or higher range can be obtained. Additional information regarding Anticalins can be found in U.S. Pat. No. 7,250,297 and WO 99/16873, both of which are hereby incorporated by reference in their entirety.

Avimers are another type of antibody mimetic technology useful in the context of the instant invention. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared to conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets. Additional information regarding Avimers can be found in US 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that can be used in the context of the instant invention. Versabodies are small proteins of 3-5 kDa with >15% cysteines, which form a high disulfide density scaffold replacing the hydrophobic core that typical proteins have. This replacement results in a protein that is smaller, is more hydrophilic (i.e., less prone to aggregation and non-specific binding), is more resistant to proteases and heat, and has a lower density of T-cell epitopes, because the residues that contribute most to MHC presentation are hydrophobic. these properties are well-known to affect immunogenicity, and together they are expected to cause a large decrease in immunogenicity.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

The above descriptions of antibody fragment and mimetic technologies is not intended to be comprehensive. A variety of additional technologies including alternative polypeptide-based technologies, such as fusions of complementarity determining regions as outlined in Qui et al., Nature Biotechnology, 25(8) 921-929 (2007), as well as nucleic acid-based technologies, such as the RNA aptamer technologies described in U.S. Pat. Nos. 5,789,157; 5,864,026; 5,712,375; 5,763,566; 6,013,443; 6,376,474; 6,613,526; 6,114,120; 6,261,774; and 6,387,620; all of which are hereby incorporated by reference, could be used in the context of the instant invention.

### Antibody Physical Properties

The antibodies used in the present invention may be characterized by the various physical properties.

The antibodies may contain one or more glycosylation sites in either the V_{L}, or V_{H}, which may result in it having increased immunogenicity or altered pK (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-antibody that does not contain variable region glycosylation. This can be achieved either by selecting antibodies that do not contain the glycosylation motif in the variable region or by mutating residues within the glycosylation motif using standard techniques.

In a preferred embodiment, the antibodies of the present disclosure do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect). The presence of isoaspartic acid can be measured using a reverse-phase HPLC test (iso-quant assay).

Each antibody will have a unique isoelectric point (pI), generally falling in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-PTK7 antibody that contains a pI value that falls in the normal range. This can be achieved either by selecting antibodies with a pI in the normal range or by mutating charged surface residues.

Each antibody will have a characteristic melting temperature, with a higher melting temperature indicating greater overall stability in vivo (Krishnamurthy R and Manning M C (2002) Curr Pharm Biotechnol 3:361-71). Generally, it is preferred that the T_{M1} (the temperature of initial unfolding) be greater than 60° C., preferably greater than 65° C., even more preferably greater than 70° C. The melting point of an antibody can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9).

In a preferred embodiment, antibodies are selected that do not rapidly degrade. Fragmentation of an antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects, which can lead to the triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation can be measured by several techniques, including size-exclusion column (SEC), high performance liquid chromatography (HPLC), and light scattering.

### Methods of Engineering Antibodies

As discussed above, the anti-PTK7 antibodies having V_{H} and V_{K} sequences disclosed herein can be used to create new anti-PTK7 antibodies by modifying the VH and/or V_{K} sequences, or the constant region(s) attached thereto. Thus, in another aspect of the invention, the structural features of an anti-PTK7 antibody of the invention, e.g. 3G8, 3G8a, 4D5, 12C6, 12C6a or 7C8, are used to create structurally related anti-PTK7 antibodies that retain at least one functional property of the antibodies of the invention, such as binding to human PTK7. For example, one or more CDR regions of 3G8, 3G8a, 4D5, 12C6, 12C6a or 7C8, or mutations thereof, can be combined recombinantly with known framework regions and/or other CDRs to create additional, recombinantly-engineered, anti-PTK7 antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-PTK7 antibody comprising:
(a) providing: (i) a heavy chain variable region antibody sequence comprising a CDR1 sequence selected from the group consisting of SEQ ID NOs: 11, 12, 13 and 14, a CDR2 sequence selected from the group consisting of SEQ ID NOs: 15, 16, 17 and 18, and/or a CDR3 sequence selected from the group consisting of SEQ ID NOs: 19, 20, 21 and 22; and/or (ii) a light chain variable region antibody sequence comprising a CDR1 sequence selected from the group consisting of SEQ ID NOs: 23, 24, 25, 26, 27 and 28, a CDR2 sequence selected from the group consisting of SEQ ID NOs: 29, 30, 31, 32, 33 and 34, and/or a CDR3 sequence selected from the group consisting of SEQ ID NOs: 35, 36, 37, 38, 39 and 40;
(b) altering at least one amino acid residue within the heavy chain variable region antibody sequence and/or the light chain variable region antibody sequence to create at least one altered antibody sequence; and
(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-PTK7 antibodies described herein, which functional properties include, but are not limited to:
(a) the antibody binds to human PTK7 with a K_{D} of 1×10⁻⁷ M or less;
(b) the antibody binds the Wilms' tumor cell line.

The functional properties of the altered antibodies can be assessed using standard assays available in the art and/or described herein, such as those set forth in the Examples (e.g., flow cytometry, binding assays).

In certain embodiments of the methods of engineering antibodies of the invention, mutations can be introduced randomly or selectively along all or part of an anti-PTK7 antibody coding sequence and the resulting modified anti-PTK7 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids molecules of the invention are those encoding the VH and VL sequences of the 3G8, 3G8a, 4D5, 12C6, 12C6a or 7C8 monoclonal antibodies. DNA sequences encoding the VH sequences of 3G8, 3G8a, 4D5, 12C6, 12C6a and 7C8 are shown in SEQ ID NOs: 41 (3G8 and 3G8a), 42 (4D5), 43 (12C6 and 12C6a) and 44 (7C8). DNA sequences encoding the VL sequences of 3G8, 3G8a, 4D5, 12C6, 12C6a and 7C8 are shown in SEQ ID NOs: 45, 46, 47, 48, 49 and 50, respectively.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against PTK7 can be generated using transgenic or transchromosomic mice carrying parts of the human immune system rather than the mouse system. These transgenic and transchromosomic mice include mice referred to herein as HuMAb mice and KM Mice™, respectively, and are collectively referred to herein as “human Ig mice.”

The HuMAb Mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al., (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise anti-PTK7 antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-PTK7 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-PTK7 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, human anti-PTK7 antibodies are prepared using a combination of human Ig mouse and phage display techniques, as described in U.S. Pat. No. 6,794,132 by Buechler et al. More specifically, the method first involves raising an anti-PTK7 antibody response in a human Ig mouse (such as a HuMab mouse or KM mouse as described above) by immunizing the mouse with one or more PTK7 antigens, followed by isolating nucleic acids encoding human antibody chains from lymphatic cells of the mouse and introducing these nucleic acids into a display vector (e.g., phage) to provide a library of display packages. Thus, each library member comprises a nucleic acid encoding a human antibody chain and each antibody chain is displayed from the display package. The library then is screened with PTK7 protein to isolate library members that specifically bind to PTK7. Nucleic acid inserts of the selected library members then are isolated and sequenced by standard methods to determine the light and heavy chain variable sequences of the selected PTK7 binders. The variable regions can be converted to full-length antibody chains by standard recombinant DNA techniques, such as cloning of the variable regions into an expression vector that carries the human heavy and light chain constant regions such that the V_{H} region is operatively linked to the C_{H} region and the V_{L} region is operatively linked to the C_{L} region.

### Immunization of Human Ig Mice

When human Ig mice are used to raise human antibodies of the invention, such mice can be immunized with a purified or enriched preparation of PTK7 antigen and/or recombinant PTK7, or a PTK7 fusion protein, as described by Lonberg, N. et al. (1994) Nature 368 (6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of PTK7 antigen can be used to immunize the human Ig mice intraperitoneally.

Detailed procedures to generate fully human monoclonal antibodies to PTK7 are described in Example 1 below. Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-PTK7 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse™ strain can be used, as described in Example 1.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The antibodies can be aliquoted and stored at −80° C.

### Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies of the invention can be tested for binding to PTK7 by, for example, standard ELISA. Briefly, microtiter plates are coated with purified PTK7 at 0.25 μg/ml in PBS, and then blocked with 5% bovine serum albumin in PBS. Dilutions of antibody (e.g., dilutions of plasma from PTK7-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with PTK7 immunogen. Hybridomas that bind with high avidity to PTK7 are subcloned and further characterized. One clone from each hybridoma, which retains the reactivity of the parent cells (by ELISA), can be chosen for making a 5-10 vial cell bank stored at −140° C., and for antibody purification.

To purify anti-PTK7 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by 0D₂₈₀ using 1.43 extinction coefficient. The monoclonal antibodies can be aliquoted and stored at −80° C.

To determine if the selected anti-PTK7 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using PTK7 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-PTK7 human IgGs can be further tested for reactivity with PTK7 antigen by Western blotting. Briefly, PTK7 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Bispecific Molecules

In another aspect, the present invention features bispecific molecules comprising an anti-PTK7 antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for PTK7 and a second binding specificity for a second target epitope. In a particular embodiment of the invention, the second target epitope is an Fc receptor, e.g., human FcγRI (CD64) or a human Fcα receptor (CD89). Therefore, the invention includes bispecific molecules capable of binding both to FcγR or FcαR expressing effector cells (e.g., monocytes, macrophages or polymorphonuclear cells (PMNs)), and to target cells expressing PTK7. These bispecific molecules target PTK7 expressing cells to effector cell and trigger Fc receptor-mediated effector cell activities, such as phagocytosis of an PTK7 expressing cells, antibody dependent cell-mediated cytotoxicity (ADCC), cytokine release, or generation of superoxide anion.

In an embodiment of the invention in which the bispecific molecule is multispecific, the molecule can further include a third binding specificity, in addition to an anti-Fc binding specificity and an anti-PTK7 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the F_{c} receptor or target cell antigen. The “anti-enhancement factor portion” can bind an F_{c} receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), Fcγ RII(CD32), and FcγRIII (CD16). In one preferred embodiment, the Fcγ receptor a human high affinity FcγRI. The human FcγRI is a 72 kDa molecule, which shows high affinity for monomeric IgG (10⁸-10⁹M⁻¹).

The production and characterization of certain preferred anti-Fcγ monoclonal antibodies are described by Fanger et al. in PCT Publication WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol 155 (10): 4996-5002 and PCT Publication WO 94/10332. The H22 antibody producing cell line was deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (Fcα RI (CD89)), the binding of which is preferably not blocked by human immunoglobulin A (IgA). The term “IgA receptor” is intended to include the gene product of one α-gene (Fcα RI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity (≈5×10⁷M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of the invention are murine, chimeric and humanized monoclonal antibodies.

The bispecific molecules of the present invention can be prepared by conjugating the constituent binding specificities, e.g., the anti-FcR and anti-PTK7 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb x mAb, mAb x Fab, Fab x F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a y counter or a scintillation counter or by autoradiography.

### Conjugates

In conjugates of this invention, the partner molecule is conjugated to an antibody by a chemical linker (sometimes referred to herein simply as “linker”). The partner molecule can be a therapeutic agent or a marker. The therapeutic agent can be, for example, a cytotoxin, a non-cytotoxic drug (e.g., an immunosuppressant), a radioactive agent, another antibody, or an enzyme. Preferably, the partner molecule is a cytotoxin. The marker can be any label that generates a detectable signal, such as a radiolabel, a fluorescent label, or an enzyme that catalyzes a detectable modification to a substrate. The antibody serves a targeting function: by binding to a target tissue or cell where its antigen is found, the antibody steers the conjugate to the target tissue or cell. There, the linker is cleaved, releasing the partner molecule to perform its desired biological function.

The ratio of partner molecules attached to an antibody can vary, depending on factors such as the amount of partner molecule employed during conjugation reaction and the experimental conditions. Preferably, the ratio of partner molecules to antibody is between 1 and 3, more preferably between 1 and 1.5. Those skilled in the art will appreciate that, while each individual molecule of antibody Z is conjugated to an integer number of partner molecules, a preparation of the conjugate may analyze for a non-integer ratio of partner molecules to antibody, reflecting a statistical average.

### Linkers

In some embodiments, the linker is a peptidyl linker, depicted herein as (L⁴)ₚ-F-(L¹)ₘ. Other linkers include hydrazine and disulfide linkers, depicted herein as (L⁴)ₚ-H-(L¹)ₘ and (L⁴)ₚ-J-(L¹)ₘ, respectively. F, H, and J are peptidyl, hydrazine, and disulfide moieties, respectively, that are cleavable to release the partner molecule from the antibody, while L¹ and L⁴ are linker groups. F, H, J, L¹, and L⁴ are more fully defined hereinbelow, along with the subscripts p and m. The preparation and use of these and other linkers are described in WO 2005/112919, the disclosure of which is incorporated herein by reference.

The use of peptidyl and other linkers in antibody-partner conjugates is described in US 2006/0004081; 2006/0024317; 2006/0247295; U.S. Pat. Nos. 6,989,452; 7,087,600; and 7,129,261; WO 2007/051081; 2007/038658; 2007/059404; and 2007/089100; all of which are incorporated herein by reference.

Additional linkers are described in U.S. Pat. No. 6,214,345; 2003/0096743; and 2003/0130189; de Groot et al., J. Med. Chem. 42, 5277 (1999); de Groot et al. J. Org. Chem. 43, 3093 (2000); de Groot et al., J. Med. Chem. 66, 8815, (2001); WO 02/083180; Carl et al., J. Med. Chem. Lett. 24, 479, (1981); Dubowchik et al., Bioorg & Med. Chem. Lett. 8, 3347 (1998), the disclosures of which are incorporated herein by reference.

In addition to connecting the antibody and the partner molecule, a linker can impart stability to the partner molecule, reduce its in vivo toxicity, or otherwise favorably affect its pharmacokinetics, bioavailability and/or pharmacodynamics. It is generally preferred that the linker is cleaved, releasing the partner molecule, once the conjugate is delivered to its site of action. Also preferably, the linkers are traceless, such that once cleaved, no trace of the linker's presence remains.

In another embodiment, the linkers are characterized by their ability to be cleaved at a site in or near a target cell such as at the site of therapeutic action or marker activity of the partner molecule. Such cleavage can be enzymatic in nature. This feature aids in reducing systemic activation of the partner molecule, reducing toxicity and systemic side effects. Preferred cleavable groups for enzymatic cleavage include peptide bonds, ester linkages, and disulfide linkages, such as the aforementioned F, H, and J moieties. In other embodiments, the linkers are sensitive to pH and are cleaved through changes in pH.

An important aspect is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. The aforecited WO 2005/112919 discloses hydrazine linkers that can be designed to cleave at a range of speeds, from very fast to very slow.

The linkers can also serve to stabilize the partner molecule against degradation while the conjugate is in circulation, before it reaches the target tissue or cell. This is a significant benefit since it prolongates the circulation half-life of the partner molecule. The linker also serves to attenuate the activity of the partner molecule so that the conjugate is relatively benign while in circulation but the partner molecule has the desired effect—for example is cytotoxic—after activation at the desired site of action. For therapeutic agent conjugates, this feature of the linker serves to improve the therapeutic index of the agent.

In addition to the cleavable peptide, hydrazine, or disulfide groups F, H, or J, respectively, one or more linker groups L¹ are optionally introduced between the partner molecule and F, H, or J, as the case may be. These linker groups L¹ may also be described as spacer groups and contain at least two functional groups. Depending on the value of the subscript m (i.e., the number of L¹ groups present) and the location of a particular group L¹, a chemical functionality of a group L¹ can bond to a chemical functionality of the partner molecule, of F, H or J, as the case may be, or of another linker group L¹ (if more than one L¹ is present). Examples of suitable chemical functionalities for spacer groups L¹ include hydroxy, mercapto, carbonyl, carboxy, amino, ketone, aldehyde, and mercapto groups.

The linkers L¹ can be a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a polyethylene glycol moiety.

Exemplary groups L¹ include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, aminal esters, nucleic acids, peptides and the like.

One function of the groups L¹ is to provide spatial separation between F, H or J, as the case may be, and the partner molecule, lest the latter interfere (e.g., via steric or electronic effects) with cleavage chemistry at F, H, or J. The groups L¹ also can serve to introduce additional molecular mass and chemical functionality into conjugate. Generally, the additional mass and functionality affects the serum half-life and other properties of the conjugate. Thus, through careful selection of spacer groups, conjugates with a range of serum half-lives can be produced. Optionally, one or more linkers L¹ can be a self-immolative group, as described hereinbelow.

The subscript m is an integer selected from 0, 1, 2, 3, 4, 5, and 6. When multiple L¹ groups are present, they can be the same or different.

L⁴ is a linker moiety that provides spatial separation between F, H, or J, as the case may be, and the antibody, lest F, H, or J interfere with the antigen binding by the antibody or the antibody interfere with the cleavage chemistry at F, H, or J. Preferably, L⁴ imparts increased solubility or decreased aggregation properties to conjugates utilizing a linker that contains the moiety or modifies the hydrolysis rate of the conjugate. As in the case of L¹, L⁴ optionally is a self immolative group. In one embodiment, L⁴ is substituted alkyl, unsubstituted alkyl, substituted aryl, unsubstituted aryl, substituted heteroalkyl, or unsubstituted heteroalkyl, any of which may be straight, branched, or cyclic. The substitutions can be, for example, a lower (C₁-C₆) alkyl, alkoxy, alkylthio, alkylamino, or dialkylamino. In certain embodiments, L⁴ comprises a non-cyclic moiety. In another embodiment, L⁴ comprises a positively or negatively charged amino acid polymer, such as polylysine or polyarginine. L⁴ can comprise a polymer such as a polyethylene glycol moiety. Additionally, L⁴ can comprise, for example, both a polymer component and a small molecule moiety.

In a preferred embodiment, L⁴ comprises a polyethylene glycol (PEG) moiety. The PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

The subscript p is 0 or 1; that is, the presence of L⁴ is optional. Where present, L⁴ has at least two functional groups, with one functional group binding to a chemical functionality in F, H, or J, as the case may be, and the other functional group binding to the antibody. Examples of suitable chemical functionalities of groups L⁴ include hydroxy, mercapto, carbonyl, carboxy, amino, ketone, aldehyde, and mercapto groups. As antibodies typically are conjugated via sulfhydryl groups (e.g., from unoxidized cysteine residues, the addition of sulfhydryl-containing extensions to lysine residues with iminothiolane, or the reduction of disulfide bridges), amino groups (e.g., from lysine residues), aldehyde groups (e.g., from oxidation of glycoside side chains), or hydroxyl groups (e.g., from serine residues), preferred chemical functionalities for attachment to the antibody are those reactive with the foregoing groups, examples being maleimide, sulfhydryl, aldehyde, hydrazine, semicarbazide, and carboxyl groups. The combination of a sulfhydryl group on the antibody and a maleimide group on L⁴ is preferred.

In some embodiments, L⁴ comprises

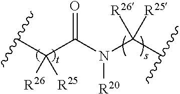

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²⁰, R²⁵, R^{25′}, R²⁶ and R^{26′} are hydrophobic. In some embodiments, R²⁰ is H or alkyl (preferably, unsubstituted lower alkyl). In some embodiments, R²⁵, R^{25′}, R²⁶ and R^{26′} are independently H or alkyl (preferably, unsubstituted C¹ to C⁴ alkyl). In some embodiments, R²⁵, R^{25′}, R²⁶ and R^{26′} are all H. In some embodiments, t is 1 and s is 1 or 2.

### Peptide Linkers (F)

As discussed above, the peptidyl linkers of the invention can be represented by the general formula: (L⁴)ₚ-F-(L¹)ₘ, wherein F represents the portion comprising the peptidyl moiety. In one embodiment, the F portion comprises an optional additional self-immolative linker L² and a carbonyl group, corresponding to a conjugate of formula (a):

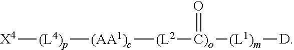

In this embodiment, L¹, L⁴, p, and m are as defined above. X⁴ is an antibody and D is a partner molecule. The subscript o is 0 or 1 and L², if present, represents a self-immolative linker. AA¹ represents one or more natural amino acids, and/or unnatural α-amino acids; c is an integer from 1 and 20. In some embodiments, c is in the range of 2 to 5 or c is 2 or 3.

In formula (a), AA¹ is linked, at its amino terminus, either directly to L⁴ or, when L⁴ is absent, directly to X⁴. In some embodiments, when L⁴ is present, L⁴ does not comprise a carboxylic acyl group directly attached to the N-terminus of (AA¹)_{c}.

In another embodiment, the F portion comprises an amino group and an optional spacer group L³ and L¹ is absent (i.e., m is 0), corresponding to a conjugate of formula (b):

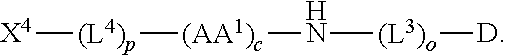

In this embodiment, X⁴, D, L⁴, AA¹, c, and p are as defined above. The subscript o is 0 or 1. L³, if present, is a spacer group comprising a primary or secondary amine or a carboxyl functional group, and either the amine of L³ forms an amide bond with a pendant carboxyl functional group of D or the carboxyl of L³ forms an amide bond with a pendant amine functional group of D.

### Self-Immolative Linkers

A self-immolative linker is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleavable by such target enzyme at the bond covalently linking the spacer moiety and the peptide moiety to thereby effect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby effect release of the drug in pharmacologically active form. See, for example, Carl et al., J. Med. Chem., 24 (3), 479-480 (1981); Carl et al., WO 81/01145 (1981); Told et al., J. Org. Chem. 67, 1866-1872 (2002); Boyd et al., WO 2005/112919; and Boyd et al., WO 2007/038658, the disclosures of which are incorporated herein by reference.

One particularly preferred self-immolative spacer may be represented by the formula (c):

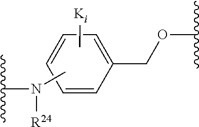

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹R²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In one preferred embodiment, i is 0.

The ether oxygen atom of the above structure is connected to a carbonyl group (not shown). The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

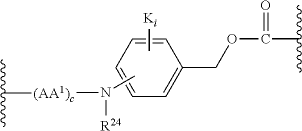

where R²⁴, AA¹, K, i, and c are as defined above.

In another embodiment, the peptide linker of formula (a) above comprises a —F-(L¹)ₘ- that comprises the structure:

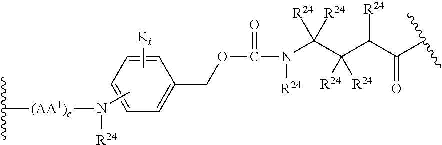

where R²⁴, AA¹, K, i, and c are as defined above.

In some embodiments, a self-immolative spacer L¹ or L² includes

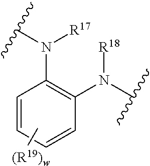

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹⁷ and R¹⁸ are independently H or alkyl (preferably, unsubstituted C₁-C₄ alkyl). Preferably, R¹⁷ and R¹⁸ are C₁₋₄ alkyl, such as methyl or ethyl. In some embodiments, w is 0. It has been found experimentally that this particular self-immolative spacer cyclizes relatively quickly.

In some embodiments, L¹ or L² includes

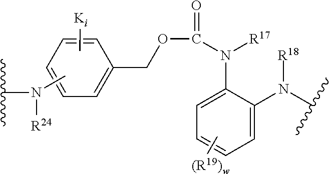

where R¹⁷, R¹⁸, R¹⁹, R²⁴, x and K are as defined above.

### Spacer Groups

The spacer group L³ is characterized by comprises a primary or secondary amine or a carboxyl functional group, and either the amine of L³ forms an amide bond with a pendant carboxyl functional group of D or the carboxyl of L³ forms an amide bond with a pendant amine functional group of D. L³ can be selected from the group consisting of substituted or unsubstituted alkyl, substituted or unsubstituted heteroalkyl, substituted or unsubstituted aryl, substituted or unsubstituted heteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

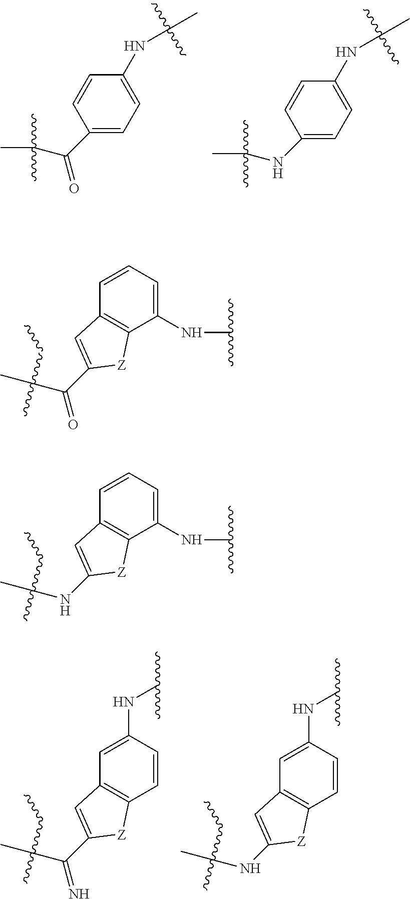

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its attachment to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

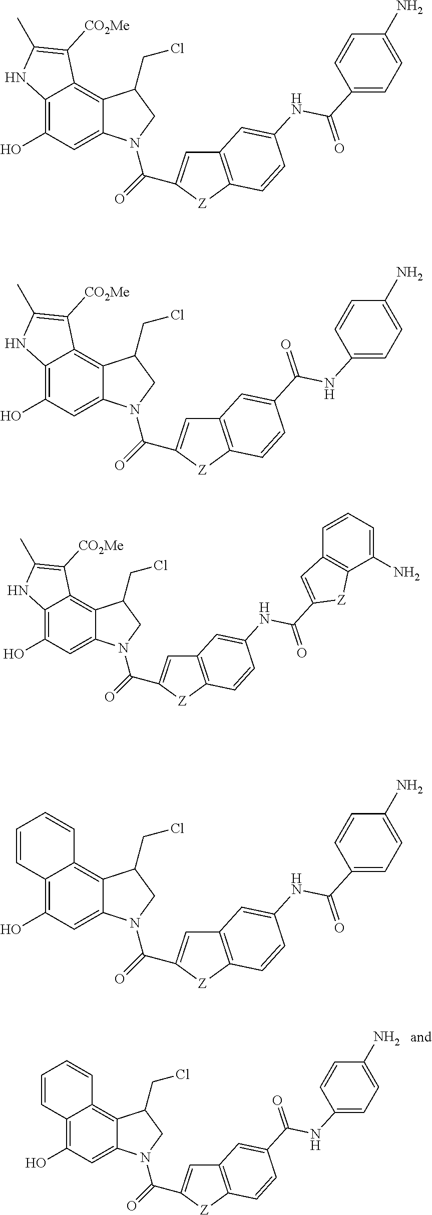

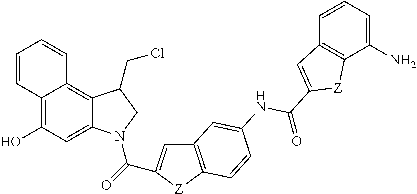

where Z is O, S or NR²³, where R²³ is H, substituted or unsubstituted alkyl, substituted or unsubstituted heteroalkyl, or acyl; and the NH₂ group on each structure reacts with (AA¹)_{c} to form -(AA¹)_{c}-NH—.

### Peptide Sequence (AA¹)_{c}

The group AA¹ represents a single amino acid or a plurality of amino acids joined together by amide bonds. The amino acids may be natural amino acids and/or unnatural α-amino acids. They may be in the L or the D configuration. In one embodiment, at least three different amino acids are used. In another embodiment, only two amino acids are used.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, citrulline, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. One amino acid that may be used in particular is citrulline, which is a precursor to arginine and is involved in the formation of urea in the liver. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When attached to a linker or conjugate of the invention, the amino acid is in the form of an “amino acid side chain”, where the carboxylic acid group of the amino acid has been replaced with a keto (C(O)) group. Thus, for example, an alanine side chain is —C(O)—CH(NH₂)—CH₃, and so forth.

The peptide sequence (AA¹)_{c} is functionally the amidification residue of a single amino acid (when c=1) or a plurality of amino acids joined together by amide bonds. The peptide sequence (AA¹)_{c} preferably is selected for enzyme-catalyzed cleavage by an enzyme in a location of interest in a biological system. For example, for conjugates that are targeted to but not internalized by a cell, a peptide is chosen that is cleaved by a protease that is in the extracellular matrix, e.g., a protease released by nearby dying cells or a tumor-associated protease, such that the peptide is cleaved extracellularly. For conjugates that are designed for internalization by a cell, the sequence (AA¹)_{c} preferably is selected for cleavage by an endosomal or lysosomal protease. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Preferably, (AA¹)_{c} contains an amino acid sequence (“cleavage recognition sequence”) that is a cleavage site by the protease. Many protease cleavage sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β-Ala-Leu-Ala-Leu (SEQ ID NO: 27). This can be combined with a stabilizing group to form succinyl-β-Ala-Leu-Ala-Leu (SEQ ID NO: 30). Other examples of suitable cleavable peptides are provided in the references cited below. Alternatively, linkers comprising a single amino acid residue can be used, as disclosed in WO 2008/103693, the disclosure of which is incorporated herein by reference.

In a preferred embodiment, the peptide sequence (AA¹)_{c} is chosen based on its ability to be cleaved by a lysosomal proteases, examples of which include cathepsins B, C, D, H, L and S. Preferably, the peptide sequence (AA¹)_{c} is capable of being cleaved by cathepsin B in vitro. Though cathepsin B is a lysosomal protease, it is believed that a certain concentration of it is found in the extracellular matrix surrounding tumor tissues.

In another embodiment, the peptide sequence (AA¹)_{c} is chosen based on its ability to be cleaved by a tumor-associated protease, such as a protease found extracellularly in the vicinity of tumor cells, examples of which include thimet oligopeptidase (TOP) and CD10. Or, the sequence (AA¹)_{c} is designed for selective cleavage by urokinase or tryptase.

As one illustrative example, CD10, also known as neprilysin, neutral endopeptidase (NEP), and common acute lymphoblastic leukemia antigen (CALLA), is a type II cell-surface zinc-dependent metalloprotease. Cleavable substrates suitable for use with CD10 include Leu-Ala-Leu and Ile-Ala-Leu.

Another illustrative example is based on matrix metalloproteases (MMP). Probably the best characterized proteolytic enzymes associated with tumors, there is a clear correlation of activation of MMPs within tumor microenvironments. In particular, the soluble matrix enzymes MMP2 (gelatinase A) and MMP9 (gelatinase B), have been intensively studied, and shown to be selectively activated during tissue remodeling including tumor growth. Peptide sequences designed to be cleaved by MMP2 and MMP9 have been designed and tested for conjugates of dextran and methotrexate (Chau et al., Bioconjugate Chem. 15:931-941 (2004)); PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, Pro-Val-Gly-Leu-Ile-Gly (SEQ. ID NO: 21), Gly-Pro-Leu-Gly-Val (SEQ. ID NO: 22), Gly-Pro-Leu-Gly-Ile-Ala-Gly-Gln (SEQ. ID NO: 23), Pro-Leu-Gly-Leu (SEQ. ID NO: 24), Gly-Pro-Leu-Gly-Met-Leu-Ser-Gln (SEQ. ID NO: 25), and Gly-Pro-Leu-Gly-Leu-Trp-Ala-Gln (SEQ. ID NO: 26). (See, e.g., the previously cited references as well as Kline et al., Mol. Pharmaceut. 1:9-22 (2004) and Liu et al., Cancer Res. 60:6061-6067 (2000).)

Yet another example is type II transmembrane serine proteases. This group of enzymes includes, for example, hepsin, testisin, and TMPRSS4. Gln-Ala-Arg is one substrate sequence that is useful with matriptase/MT-SP1 (which is over-expressed in breast and ovarian cancers) and Leu-Ser-Arg is useful with hepsin (over-expressed in prostate and some other tumor types). (See, e.g., Lee et. al., J. Biol. Chem. 275:36720-36725 and Kurachi and Yamamoto, Handbook of Proteolytic Enzymes Vol. 2, 2^{nd} edition (Barrett A J, Rawlings N D & Woessner J F, eds) pp. 1699-1702 (2004).)

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu (SEQ ID NO: 27), Gly-Phe-Leu-Gly (SEQ. ID NO: 28), Val-Ala, Leu-Leu-Gly-Leu (SEQ ID NO: 29), Leu-Asn-Ala, and Lys-Leu-Val. Preferred peptides sequences are Val-Cit and Val-Lys.

In another embodiment, the amino acid located the closest to the drug moiety is selected from the group consisting of: Ala, Asn, Asp, Cit, Cys, Gln, Glu, Gly, Ile, Leu, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Glu, Gly, Ile, Leu, Met, Phe, Pro, Ser, Thr, Trp, Tyr, and Val.

One of skill in the art can readily evaluate an array of peptide sequences to determine their utility in the present invention without resort to undue experimentation. See, for example, Zimmerman, M., et al., (1977) Analytical Biochemistry 78:47-51; Lee, D., et al., (1999) Bioorganic and Medicinal Chemistry Letters 9:1667-72; and Rano, T. A., et al., (1997) Chemistry and Biology 4:149-55.

A conjugate of this invention may optionally contain two or more linkers. These linkers may be the same or different. For example, a peptidyl linker may be used to connect the drug to the ligand and a second peptidyl linker may attach a diagnostic agent to the complex. Other uses for additional linkers include linking analytical agents, biomolecules, targeting agents, and detectable labels to the antibody-partner complex.

### Hydrazine Linkers (H)

In another embodiment, the conjugate of the invention comprises a hydrazine self-immolative linker, wherein the conjugate has the structure:

X⁴-(L⁴)ₚ-H-(L¹)ₘ-D

wherein D, L¹, L⁴, p, m, and X⁴ are as defined above and described further herein, and H is a linker comprising the structure:

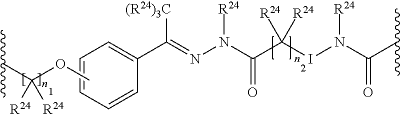

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

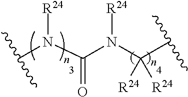

wherein n₃ is 0 or 1, with the proviso that when n₃ is 0, n₂ is not 0; and n₄ is 1, 2, or 3.

In one embodiment, the substitution on the phenyl ring is a para substitution. In preferred embodiments, n₁ is 2, 3, or 4 or n₁ is 3. In preferred embodiments, n₂ is 1. In preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Another hydrazine structure, H, has the formula:

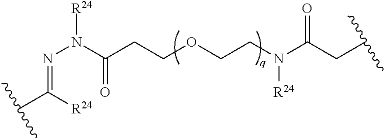

where q is 0, 1, 2, 3, 4, 5, or 6; and each R²⁴ is a member independently selected from the group consisting of H, substituted alkyl, unsubstituted alkyl, substituted heteroalkyl, and unsubstituted heteroalkyl. This hydrazine structure can also form five-, six-, or seven-membered rings and additional components can be added to form multiple rings.

The preparation, cleavage chemistry and cyclization kinetics of the various hydrazine linkers is disclosed in WO 2005/112919, the disclosure of which is incorporated herein by reference.

### Disulfide Linkers (J)

In yet another embodiment, the linker comprises an enzymatically cleavable disulfide group. In one embodiment, the invention provides a cytotoxic antibody-partner compound having a structure according to Formula (d):

X⁴L⁴ₚJL¹ₘD

wherein D, L¹, L⁴, p, m, and X⁴ are as defined above and described further herein, and J is a disulfide linker comprising a group having the structure:

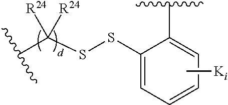

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0, 1, 2, 3, or 4; and d is an integer of 0, 1, 2, 3, 4, 5, or 6.

The aromatic ring of a disulfide linker can be substituted with one or more “K” groups. A “K” group is a substituent that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In a specific embodiment, i is 0.

In a preferred embodiment, the linker comprises an enzymatically cleavable disulfide group of the following formula:

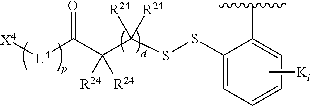

wherein L⁴, X⁴, p, and R²⁴ are as described above, and d is 0, 1, 2, 3, 4, 5, or 6. In a particular embodiment, d is 1 or 2.

A more specific disulfide linker is shown in the formula below:

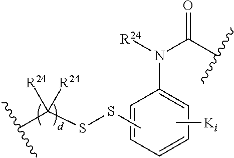

Preferably, d is 1 or 2 and each K is H.

Another disulfide linker is shown in the formula below:

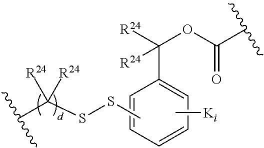

Preferably, d is 1 or 2 and each K is H.

In various embodiments, the disulfides are ortho to the amine. In another specific embodiment, a is 0. In preferred embodiments, R²⁴ is independently selected from H and CH₃.

The preparation and use of disulfide linkers such as those described above is disclosed in WO 2005/112919, the disclosure of which is incorporated herein by reference.

For further discussion of types of cytotoxins, linkers and the conjugation of therapeutic agents to antibodies, see also U.S. Pat. No. 7,087,600; U.S. Pat. No. 6,989,452; U.S. Pat. No. 7,129,261; US 2006/0004081; US 2006/0247295; WO 02/096910; WO 2007/051081; WO 2005/112919; WO 2007/059404; WO 2008/083312; WO 2008/103693; Saito et al. (2003) Adv. Drug Deliv. Rev. 55:199-215; Trail et al. (2003) Cancer Immunol. Immunother. 52:328-337; Payne. (2003) Cancer Cell 3:207-212; Allen (2002) Nat. Rev. Cancer 2:750-763; Pastan and Kreitman (2002) Curr. Opin. Investig. Drugs 3:1089-1091; Senter and Springer (2001) Adv. Drug Deliv. Rev. 53:247-264, each of which is hereby incorporated by reference.

### Cytotoxins as Partner Molecules

In one aspect, the present invention features an antibody conjugated to a partner molecule, such as a cytotoxin, a drug (e.g., an immunosuppressant) or a radiotoxin. Such conjugates are also referred to as “immunotoxins.” A cytotoxin or cytotoxic agent includes any agent that is detrimental to (e.g., kills) cells. Herein, “cytotoxin” includes compounds that are in a prodrug form and are converted in vivo to the actual toxic species.

Examples of partner molecules of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Examples of partner molecules also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, tubulysin, dibromomannitol, streptozotocin, mitomycin C, cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other preferred examples of partner molecules that can be conjugated to an antibody of the invention include calicheamicins, maytansines and auristatins, and derivatives thereof.

Preferred examples of partner molecule are analogs and derivatives of CC-1065 and the structurally related duocarmycins. Despite its potent and broad antitumor activity, CC-1065 cannot be used in humans because it causes delayed death in experimental animals, prompting a search for analogs or derivatives with a better therapeutic index.

Many analogues and derivatives of CC-1065 and the duocarmycins are known in the art. The research into the structure, synthesis and properties of many of the compounds has been reviewed. See, for example, Boger et al., Angew. Chem. Int. Ed. Engl. 35: 1438 (1996); and Boger et al., Chem. Rev. 97: 787 (1997). Other disclosures relating to CC-1065 analogs or derivatives include: U.S. Pat. No. 5,101,038; U.S. Pat. No. 5,641,780; U.S. Pat. No. 5,187,186; U.S. Pat. No. 5,070,092; U.S. Pat. No. 5,703,080; U.S. Pat. No. 5,070,092; U.S. Pat. No. 5,641,780; U.S. Pat. No. 5,101,038; U.S. Pat. No. 5,084,468; U.S. Pat. No. 5,739,350; U.S. Pat. No. 4,978,757, U.S. Pat. No. 5,332,837 and U.S. Pat. No. 4,912,227; WO 96/10405; and EP 0,537,575 A1

In a particularly preferred aspect, the partner molecule is a CC-1065/duocarmycin analog having a structure according to the following formula (e):

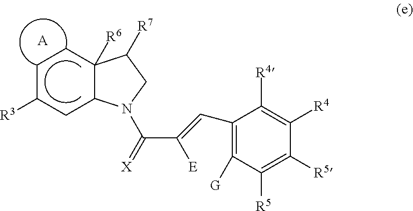

in which ring system A is a member selected from substituted or unsubstituted aryl substituted or unsubstituted heteroaryl and substituted or unsubstituted heterocycloalkyl groups. Exemplary ring systems A include phenyl and pyrrole.

The symbols E and G are independently selected from H, substituted or unsubstituted alkyl, substituted or unsubstituted heteroalkyl, a heteroatom, a single bond or E and G are optionally joined to form a ring system selected from substituted or unsubstituted aryl, substituted or unsubstituted heteroaryl and substituted or unsubstituted heterocycloalkyl.

The symbol X represents a member selected from O, S and NR²³. R²³ is a member selected from H, substituted or unsubstituted alkyl, substituted or unsubstituted heteroalkyl, and acyl.

The symbol R³ represents a member selected from (═O), SR¹¹, NHR¹¹ and OR¹¹, in which R¹¹ is H, substituted or unsubstituted alkyl, substituted or unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² or SiR¹²R¹³R¹⁴. The symbols R¹², R¹³, and R¹⁴ independently represent H, substituted or unsubstituted alkyl, substituted or unsubstituted heteroalkyl and substituted or unsubstituted aryl, where R¹² and R¹³ together with the nitrogen or carbon atom to which they are attached are optionally joined to form a substituted or unsubstituted heterocycloalkyl ring system having from 4 to 6 members, optionally containing two or more heteroatoms.

R⁴, R⁴′, R⁵ and R⁵′ are members independently selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20, or any adjacent pair of R⁴, R⁴′, R⁵ and R⁵′, together with the carbon atoms to which they are attached, are joined to form a substituted or unsubstituted cycloalkyl or heterocycloalkyl ring system having from 4 to 6 members. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. One exemplary structure is aniline.

One of R³, R⁴, R⁴′, R⁵, and R⁵′ joins the cytotoxin to a linker or enzyme cleavable substrate of the present invention, as described herein, for example to L¹ or L³, if present or to F, H, or J.

R⁶ is a single bond which is either present or absent. When R⁶ is present, R⁶ and R⁷ are joined to form a cyclopropyl ring. R⁷ is CH₂—X¹ or —CH₂—. When R⁷ is —CH₂— it is a component of the cyclopropane ring. The symbol X¹ represents a leaving group such as a halogen, for example Cl, Br or F. The combinations of R⁶ and R⁷ are interpreted in a manner that does not violate the principles of chemical valence.

X¹ may be any leaving group. Useful leaving groups include, but are not limited to, halogens, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. Particular halogens useful as leaving groups are F, Cl and Br.

The curved line within the six-membered ring indicates that the ring may have one or more degrees of unsaturation, and it may be aromatic. Thus, ring structures such as those set forth below, and related structures, are within the scope of Formula (f):

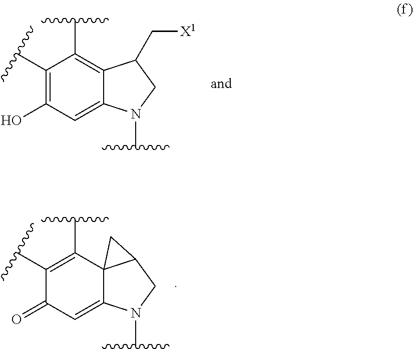

In one embodiment, R¹¹ includes a moiety, X⁵, that does not self-cyclize and links the drug to L¹ or L³, if present, or to F, H, or J. The moiety, X⁵, is preferably cleavable using an enzyme and, when cleaved, provides the active drug. As an example, R¹¹ can have the following structure (with the right side coupling to the remainder of the drug):

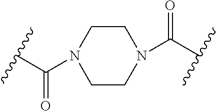

In some embodiments, at least one of R⁴, R⁴′, R⁵, and R⁵′ links said drug to L¹, if present, or to F, H, J, or X², and R³ is selected from SR¹¹, NHR¹¹ and OR¹¹. R¹¹ is selected from —SO(OH)₂, —PO(OH)₂, -AAₙ, —Si(CH₃)₂C(CH₃)₃, —C(O)OPhNH(AA)ₘ,

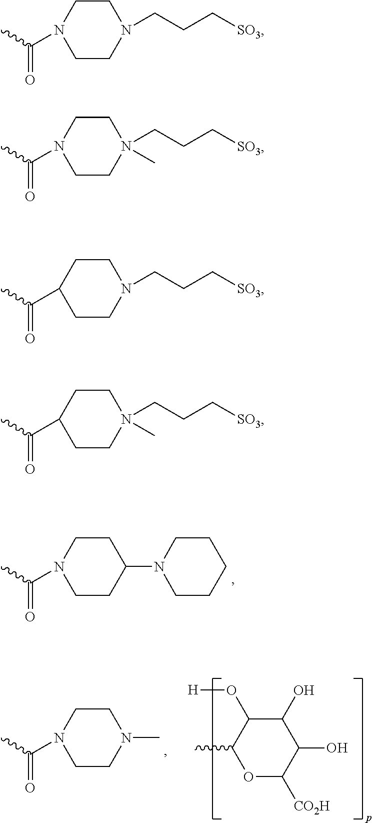

or any other sugar or combination of sugars

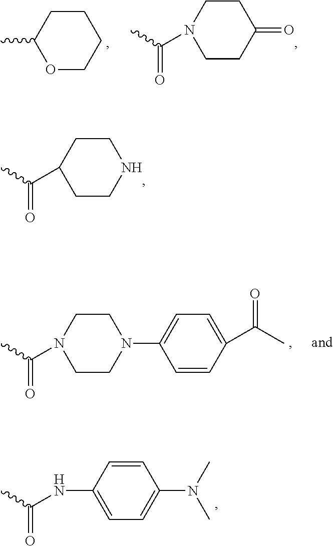

and pharmaceutically acceptable salts thereof, where n is any integer in the range of 1 to 10, m is any integer in the range of 1 to 4, p is any integer in the range of 1 to 6, and AA is any natural or non-natural amino acid. Where the compound of formula (e) is conjugated via R⁴, R⁴′, R⁵, or R⁶, R³ preferably comprises a cleavable blocking group whose presence blocks the cytotoxic activity of the compound but is cleavable under conditions found at the intended site of action by a mechanism different from that for cleavage of the linker conjugating the cytotoxin to the antibody. In this way, if there is adventitious cleavage of the conjugate in the plasma, the blocking group attenuates the cytotoxicity of the released cytotoxin. For instance, if the conjugate has a hydrazone or disulfide linker, the blocking group can be an enzymatically cleavable amide. Or, if the linker is a peptidyl one cleavable by a protease, the blocking group can be an ester or carbamate cleavable by a carboxyesterase.

For example, in a preferred embodiment, D is a cytotoxin having a structure (j):

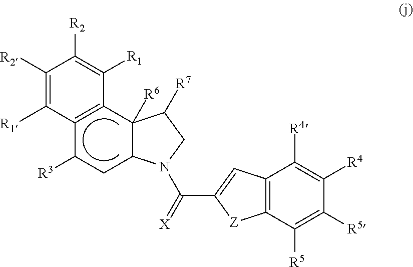

In this structure, R³, R⁶, R⁷, R⁴, R⁴′, R⁵, R⁵′ and X are as described above for Formula (e). Z is a member selected from O, S and NR²³, where R²³ is a member selected from H, substituted or unsubstituted alkyl, substituted or unsubstituted heteroalkyl, and acyl.

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R² is H, or substituted or unsubstituted lower alkyl or unsubstituted heteroalkyl or cyano or alkoxy; and R²′ is H, or substituted or unsubstituted lower alkyl or unsubstituted heteroalkyl.

One of R³, R⁴, R⁴′, R⁵, or R⁵′ links the cytotoxin to L¹ or L³, if present, or to F, H, or J.

A further embodiment has the formula:

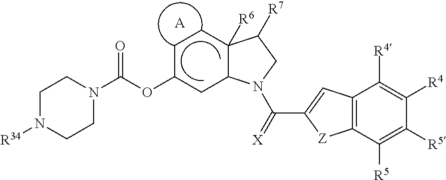

In this structure, A, R⁶, R⁷, X, R⁴, R⁴′, R⁵, and R⁵′ are as described above for Formula (e). Z is a member selected from O, S and NR²³, where R²³ is a member selected from H, substituted or unsubstituted alkyl, substituted or unsubstituted heteroalkyl, and acyl;

R³⁴ is C(═O)R³³ or C₁-C₆ alkyl, where R³³ is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms.

Preferably, A is substituted or unsubstituted phenyl or substituted or unsubstituted pyrrole. Further, any selection of substituents described herein for R¹¹ is also applicable to R³³.

A preferred partner molecule has a structure represented by formula (I)

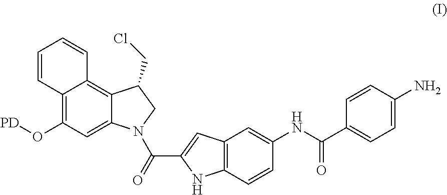

In formula (I), PD represents a prodrugging group (sometimes also referred to as a protecting group). Compound (I) is hydrolyzed in situ (preferably enzymatically) to release the compound of formula (II). As those skilled in the art will recognize, compound (II) belongs to the class of compounds known as CBI compounds (Boger et al., J. Org. Chem. 2001, 66, 6654-6661 and Boger et al., US 2005/0014700 A1 (2005). CBI compounds are converted in situ (or, when administered to a patient, in vivo) to their cyclopropyl derivatives such as compound (III), bind to the minor groove of DNA, and then alkylate DNA on an adenine group, with the cyclopropyl derivative believed to be the actual alkylating species.

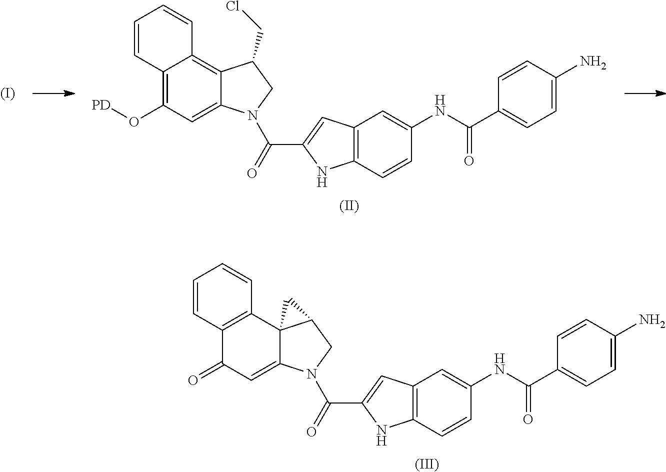

Non-limiting examples of suitable prodrugging groups PD include esters, carbamates, phosphates, and glycosides, as illustrated following:

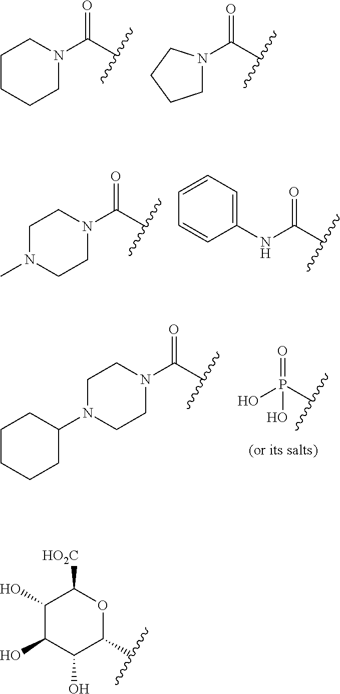

Preferred prodrugging groups PD are carbamates (exemplified by the first five structures above), which are hydrolyzable by carboxyesterases; phosphates (the sixth structure above), which are hydrolyzable by alkaline phosphatase, and β-glucuronic acid derivatives, which are hydrolyzable by β-glucuronidase. A specific preferred partner molecule is a carbamate prodrugged one, represented by formula (IV):

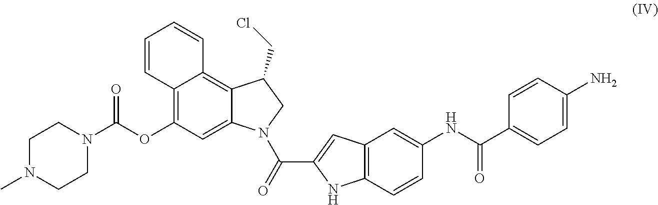

### Markers as Partner Molecules

Where the partner molecule is a marker, it can be any moiety having or generating a detectable physical or chemical property, thereby indicating its presence in a particular tissue or cell. Markers (sometimes also called reporter groups) have been well developed in the area of immunoassays, biomedical research, and medical diagnosis. A marker may be detected by spectroscopic, photochemical, biochemical, immunochemical, electrical, optical or chemical means. Examples include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The marker is preferably a member selected from the group consisting of radioactive isotopes, fluorescent agents, fluorescent agent precursors, chromophores, enzymes and combinations thereof. Examples of suitable enzymes are horseradish peroxidase, alkaline phosphatase, β-galactosidase, and glucose oxidase. Fluorescent agents include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Markers can be attached by indirect means: a ligand molecule (e.g., biotin) is covalently bound to an antibody. The ligand then binds to another molecule (e.g., streptavidin), which is either inherently detectable or covalently bound to a signal system, such as a detectable enzyme, a fluorescent compound, or a chemiluminescent compound.

### Examples of Conjugates

Specific examples of partner molecule-linker combinations suitable for conjugation to an antibody of this invention are shown following:

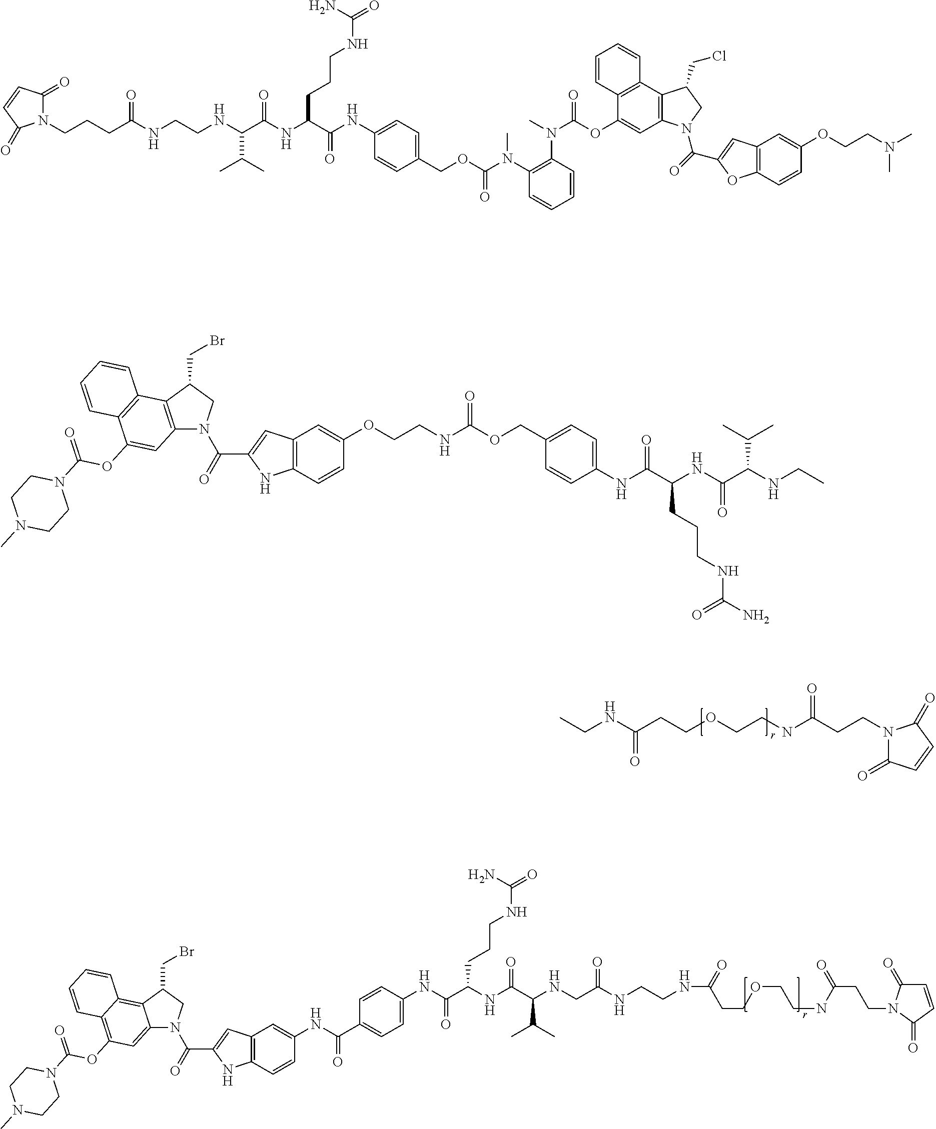

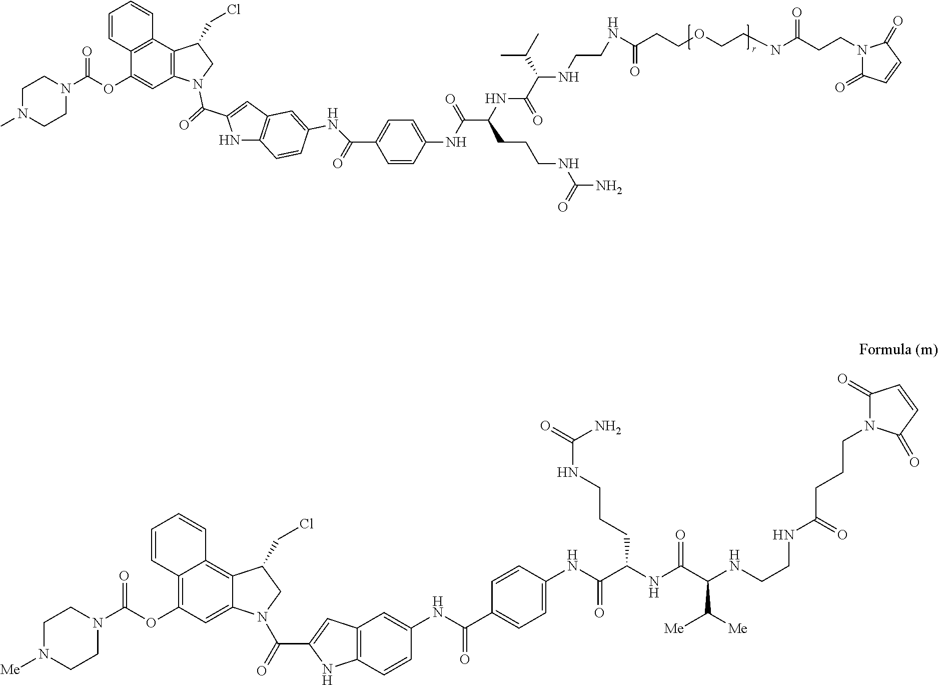

Formula (o) is shown below:

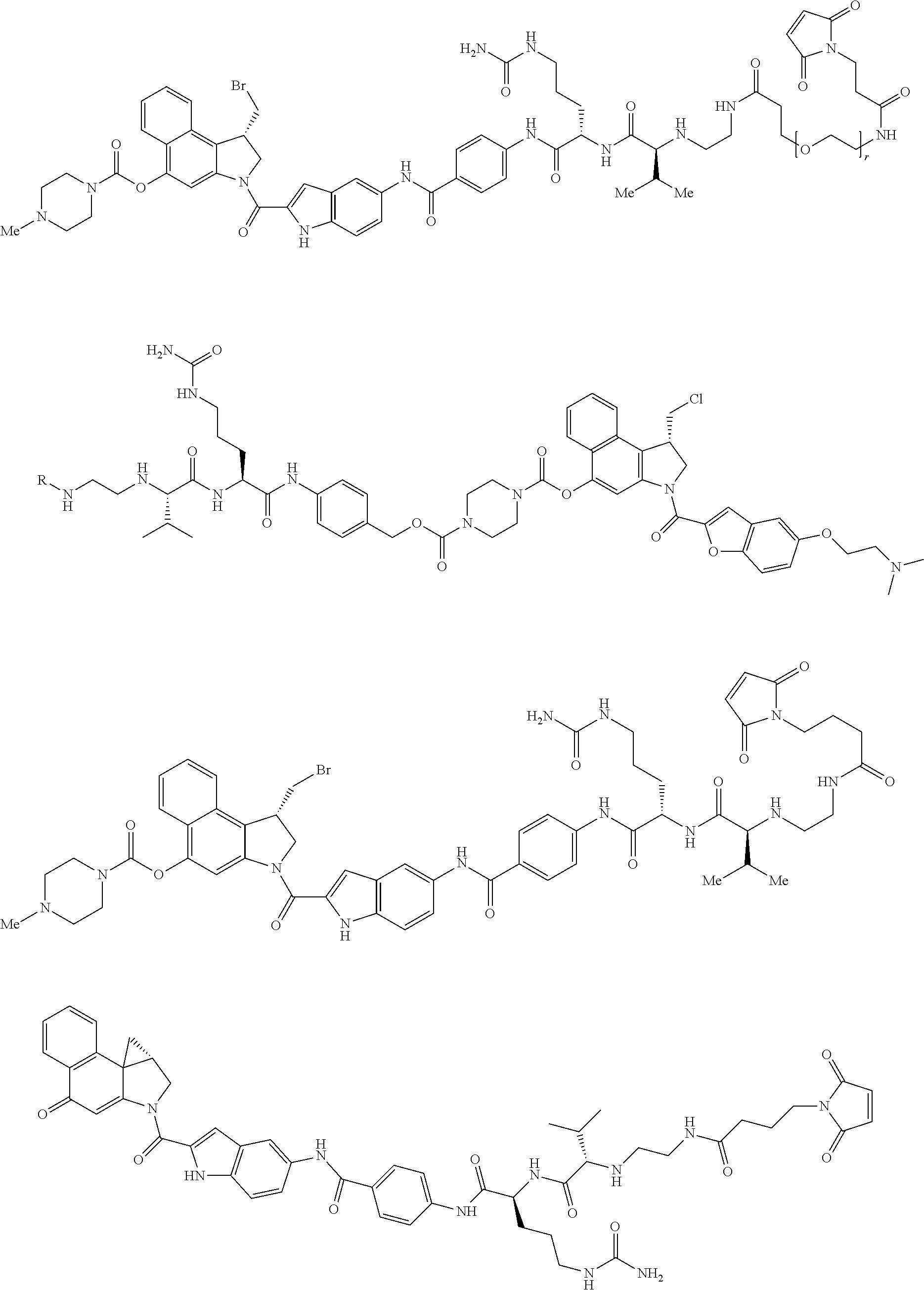

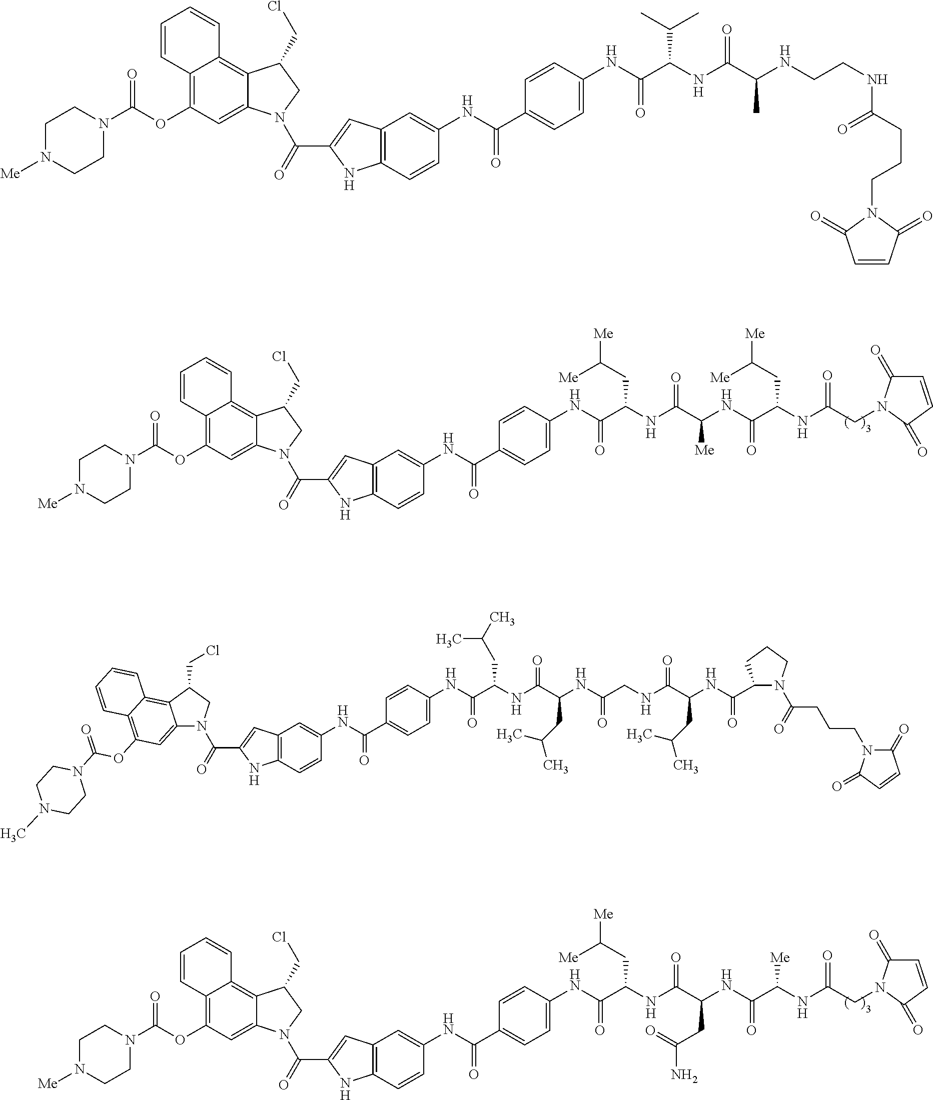

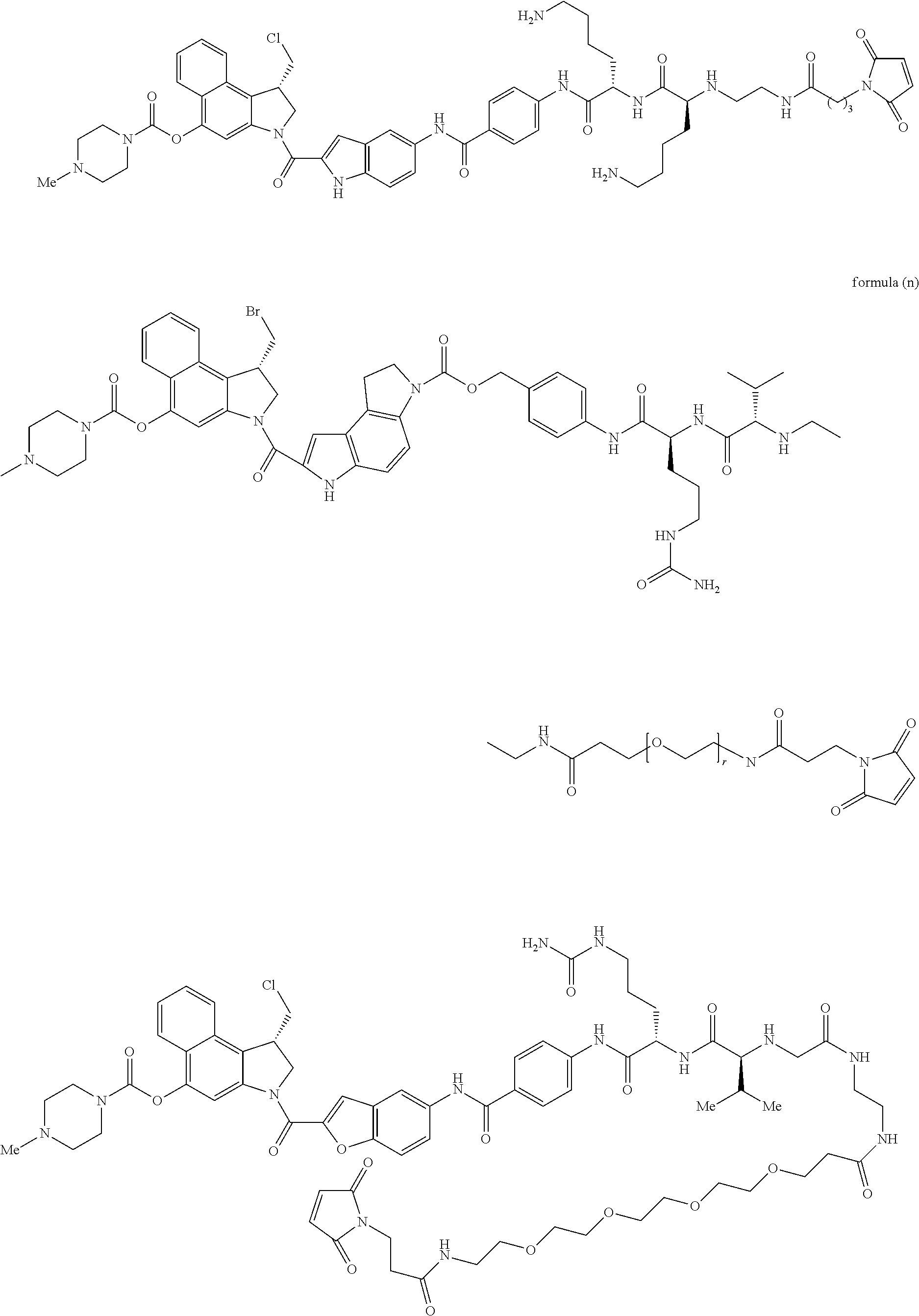

Formula (p) is shown below:

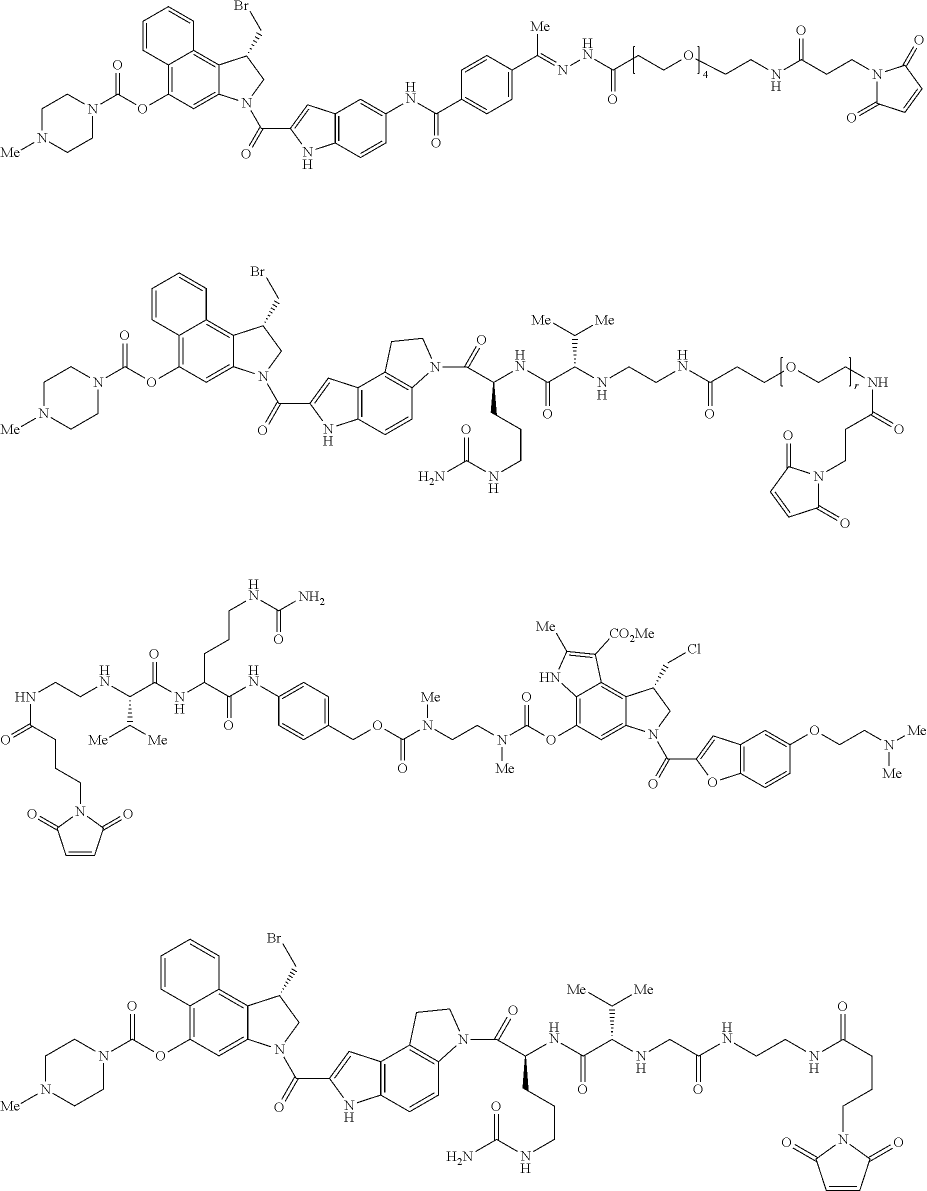

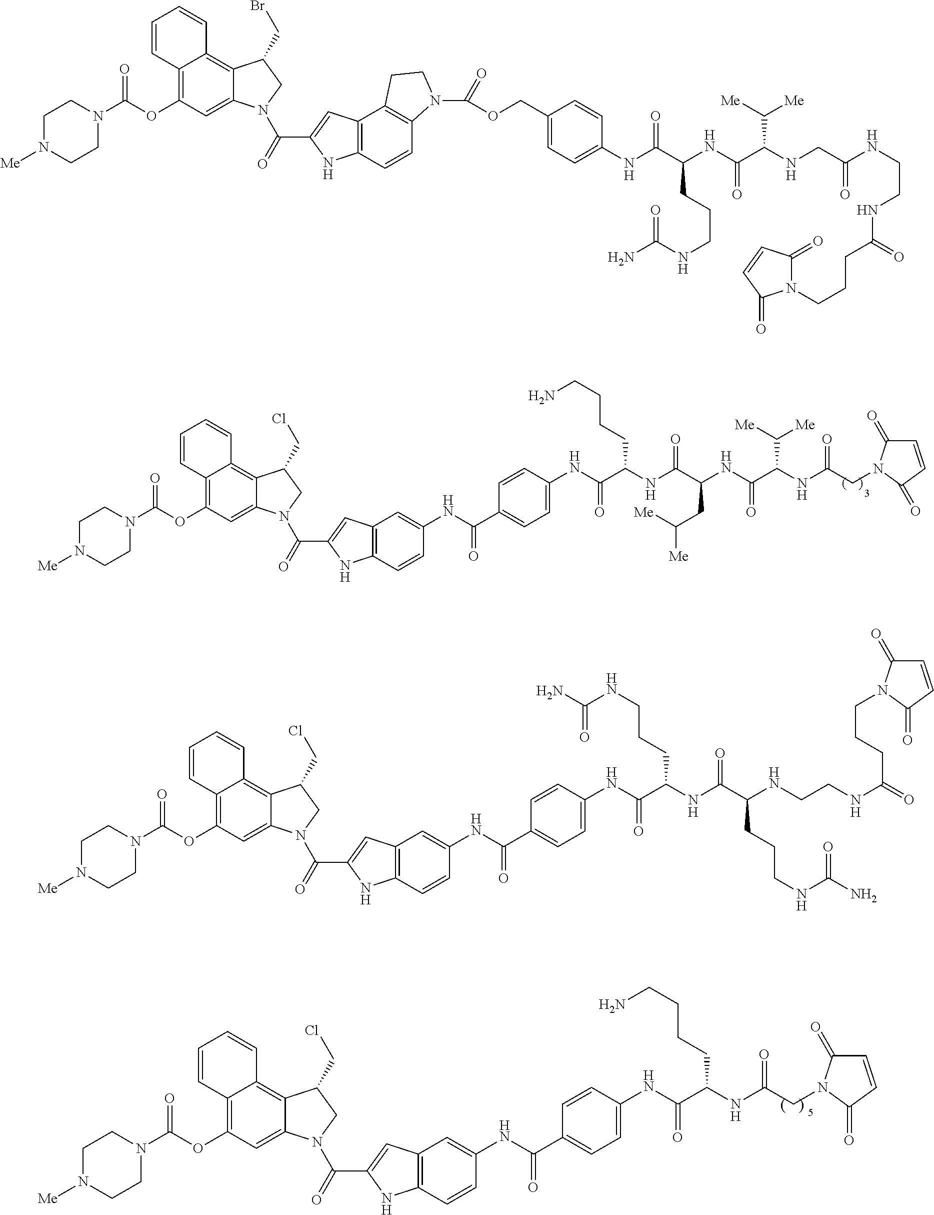

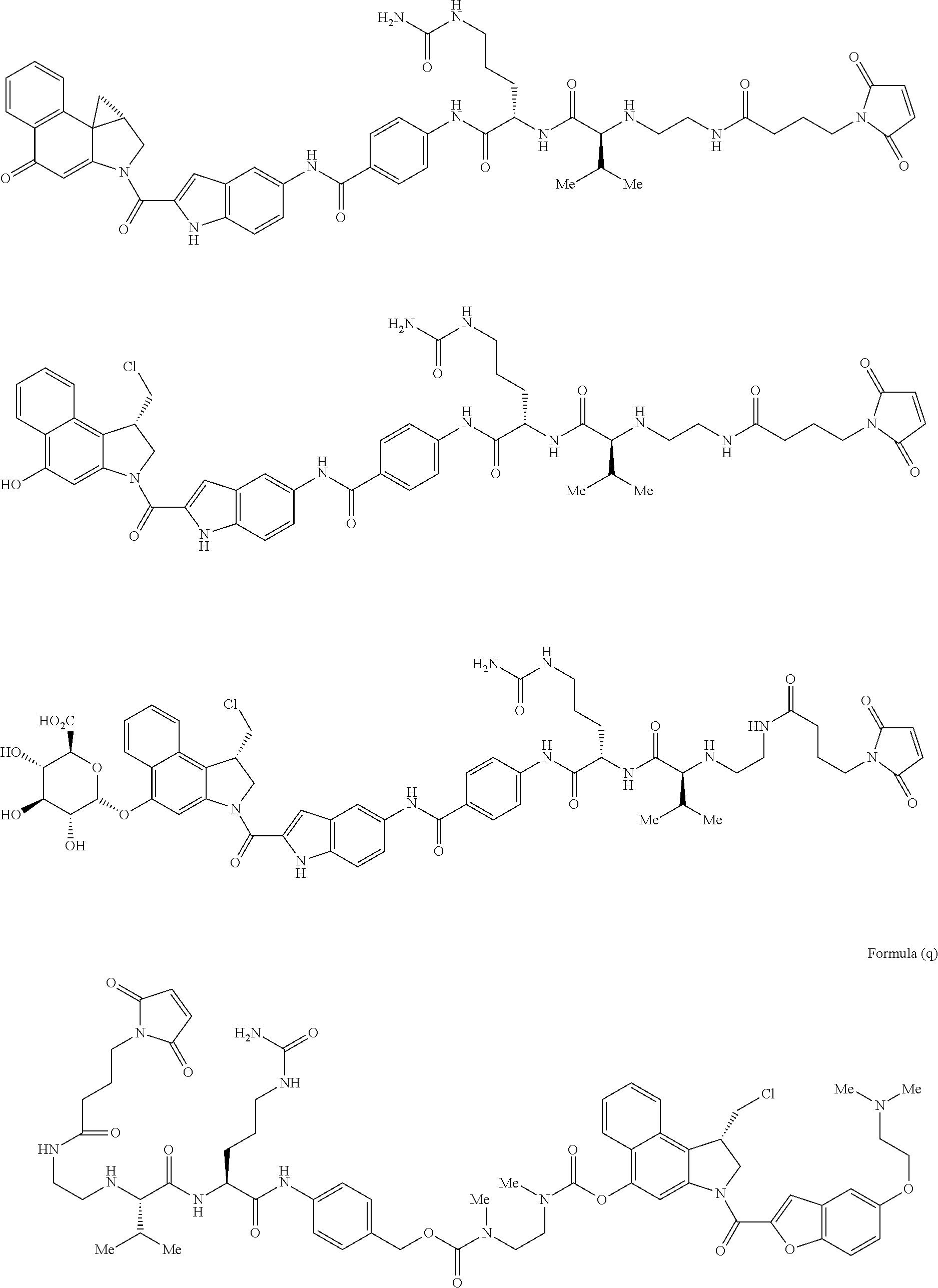

In the foregoing compounds, where the subscript r is present in a formula, it is an integer in the range of 0 to 24. R, wherever it occurs, is

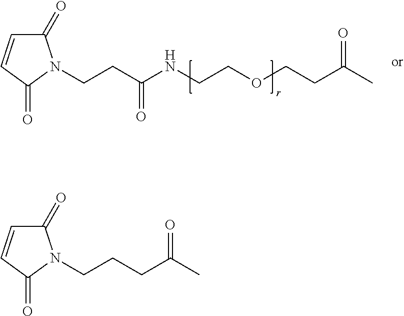

Each of the foregoing compounds has a maleimide group and is ready for conjugation to an antibody via a sulfhydryl group thereon.

### Pharmaceutical Compositions

In another aspect, the present invention provides a pharmaceutical composition containing a conjugate of the present invention formulated together with a pharmaceutically acceptable carrier and, optionally, other active or inactive ingredients.

Pharmaceutical compositions of the invention also can be administered in combination therapy with other agents. For example, the combination therapy can include a conjugate of the present invention combined with at least one other anti-inflammatory or immunosuppressant agent. Examples of therapeutic agents that can be used in combination therapy are described in greater detail below.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable carriers include water, ethanol, polyols (such as glycerol, propylene glycol, polyethylene glycol, and the like), and mixtures thereof, vegetable oils such as olive oil, and injectable organic esters, such as ethyl oleate. Proper fluidity can be maintained by the use of coating materials, such as lecithin, by the maintenance of the required particle size in the case of dispersions, and by the use of surfactants.

The compositions may also contain adjuvants such as preservatives, wetting agents, emulsifying agents and dispersing agents. Prevention of presence of microorganisms may be ensured both by sterilization and by the inclusion of antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, e.g., water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient that can be combined with a carrier to produce a single dosage form will vary depending upon the subject being treated and the particular mode of administration and will generally be that amount of the composition that produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of a conjugate, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for conjugate of the invention include 1 mg/kg body weight or 3 mg/kg body weight via intravenous administration, with the conjugate being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma conjugate concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In some embodiments, patient doses that retard or inhibit tumor growth can be 1 μmol/kg/day or less. For example, the patient doses can be 0.9, 0.6, 0.5, 0.45, 0.3, 0.2, 0.15, or 0.1 μmmol/kg/day or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

Actual dosage levels may be varied so as to obtain an amount of the active ingredient effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion, the duration of the treatment, other drugs, compounds and/or materials used in combination with the particular compositions employed, the age, sex, weight, condition, general health and prior medical history of the patient, and like factors.

A “therapeutically effective dosage” of a conjugate of the invention preferably results in a decrease in severity of disease symptoms, an increase in frequency and duration of disease symptom-free periods, and/or a prevention of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a conjugate to inhibit tumor growth can be evaluated in an animal model system predictive of efficacy in human tumors. Alternatively, this property of a composition can be evaluated by examining its ability to inhibit cell growth, such ability being measurable in vitro by assays known to the skilled practitioner. A therapeutically effective amount of a therapeutic compound can decrease tumor size, or otherwise ameliorate symptoms in a subject. One of ordinary skill in the art can determine such amounts based on such factors as the subject's size, the severity of symptoms, and the particular composition or route of administration selected.

A conjugate of this invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically.

The active compounds can be prepared with carriers that will protect them against premature release, such as a controlled release formulation, implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as disclosed in U.S. Pat. No. 5,399,163; 5,383,851; 5,312,335; 5,064,413; 4,941,880; 4,790,824; or 4,596,556. Examples of other suitable devices include those disclosed in: U.S. Pat. No. 4,487,603; U.S. Pat. No. 4,486,194; U.S. Pat. No. 4,447,233; U.S. Pat. No. 4,447,224; U.S. Pat. No. 4,439,196; and U.S. Pat. No. 4,475,196. These patents are incorporated herein by reference.

In certain embodiments, the conjugates of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V.V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Uses and Methods of the Invention

The antibodies, antibody compositions and methods of the present invention have numerous in vitro and in vivo diagnostic and therapeutic utilities involving the diagnosis and treatment of PTK7 mediated disorders. In a preferred embodiment, the antibodies of the present invention are human antibodies. For example, these molecules can be administered to cells in culture, in vitro or ex vivo, or to human subjects, e.g., in vivo, to treat, prevent and to diagnose a variety of disorders. As used herein, the term “subject” is intended to include human and non-human animals. Non-human animals includes all vertebrates, e.g., mammals and non-mammals, such as non-human primates, sheep, dogs, cats, cows, horses, chickens, amphibians, and reptiles. Preferred subjects include human patients having disorders mediated by PTK7 activity. The methods are particularly suitable for treating human patients having a disorder associated with aberrant PTK7 expression. When antibodies to PTK7 are administered together with another agent, the two can be administered in either order or simultaneously.

Given the specific binding of the antibodies of the invention for PTK7, the antibodies of the invention can be used to specifically detect PTK7 expression on the surface of cells and, moreover, can be used to purify PTK7 via immunoaffinity purification.

The invention further provides methods for detecting the presence of human PTK7 antigen in a sample, or measuring the amount of human PTK7 antigen, comprising contacting the sample, and a control sample, with a human monoclonal antibody, or an antigen binding portion thereof, which specifically binds to human PTK7, under conditions that allow for formation of a complex between the antibody or portion thereof and human PTK7. The formation of a complex is then detected, wherein a difference complex formation between the sample compared to the control sample is indicative the presence of human PTK7 antigen in the sample.

PTK7 is expressed in colon carcinoma derived cell lines but not found to be expressed in human adult colon tissues (Mossie et al. (1995) Oncogene 11:2179-84). PTK7 expression was also seen in some melanoma cell lines and melanoma biopsies (Easty, et al. (1997) Int. J. Cancer 71:1061-5). In addition, PTK7 was found to be highly overexpressed in acute myeloid leukemia samples (Muller-Tidow et al., (2004) Clin. Cancer Res. 10:1241-9). An anti-PTK7 antibody may be used alone to inhibit the growth of cancerous tumors. Alternatively, an anti-PTK7 antibody may be used in conjunction with other immunogenic agents, standard cancer treatments or other antibodies, as described below.

Preferred cancers whose growth may be inhibited using the antibodies of the invention include cancers typically responsive to immunotherapy. Non-limiting examples of preferred cancers for treatment include colon cancer (including small intestine cancer), lung cancer, breast cancer, pancreatic cancer, melanoma (e.g., metastatic malignant melanoma), acute myeloid leukemia, kidney cancer, bladder cancer, ovarian cancer and prostate cancer. Examples of other cancers that may be treated using the methods of the invention include renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma and combinations of said cancers.

Furthermore, given the expression of PTK7 on various tumor cells, the human antibodies, antibody compositions and methods of the present invention can be used to treat a subject with a tumorigenic disorder, e.g., a disorder characterized by the presence of tumor cells expressing PTK7 including, for example, colon cancer (including small intestine cancer), melanoma (e.g., metastatic malignant melanoma), acute myeloid leukemia, lung cancer, breast cancer, bladder cancer, pancreatic cancer, ovarian cancer and prostate cancer. Examples of other subjects with a tumorigenic disorder include subjects having renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma and combinations of said cancers.

Accordingly, in one embodiment, the invention provides a method of inhibiting growth of tumor cells in a subject, comprising administering to the subject a therapeutically effective amount of an anti-PTK7 antibody or antigen-binding portion thereof. Preferably, the antibody is a human anti-PTK7 antibody (such as any of the human anti-human PTK7 antibodies described herein). Additionally or alternatively, the antibody may be a chimeric or humanized anti-PTK7 antibody.

In one embodiment, the antibodies (e.g., human monoclonal antibodies, multispecific and bispecific molecules and compositions) of the invention can be used to detect levels of PTK7 or levels of cells which contain PTK7 on their membrane surface, which levels can then be linked to certain disease symptoms. Alternatively, the antibodies can be used to inhibit or block PTK7 function which, in turn, can be linked to the prevention or amelioration of certain disease symptoms, thereby implicating PTK7 as a mediator of the disease. This can be achieved by contacting an experimental sample and a control sample with the anti-PTK7 antibody under conditions that allow for the formation of a complex between the antibody and PTK7. Any complexes formed between the antibody and PTK7 are detected and compared in the experimental sample and the control.

In another embodiment, the antibodies (e.g., human antibodies, multispecific and bispecific molecules and compositions) of the invention can be initially tested for binding activity associated with therapeutic or diagnostic use in vitro. For example, compositions of the invention can be tested using the flow cytometric assays described in the Examples below.

The antibodies (e.g., human antibodies, multispecific and bispecific molecules, immunoconjugates and compositions) of the invention have additional utility in therapy and diagnosis of PTK7-related diseases. For example, the human monoclonal antibodies, the multispecific or bispecific molecules and the immunoconjugates can be used to elicit in vivo or in vitro one or more of the following biological activities: to inhibit the growth of and/or kill a cell expressing PTK7; to mediate phagocytosis or ADCC of a cell expressing PTK7 in the presence of human effector cells; or to block PTK7 ligand binding to PTK7.

In a particular embodiment, the antibodies (e.g., human antibodies, multispecific and bispecific molecules and compositions) are used in vivo to treat, prevent or diagnose a variety of PTK7-related diseases. Examples of PTK7-related diseases include, among others, colon cancer (including small intestine cancer), melanoma (e.g., metastatic malignant melanoma), acute myeloid leukemia, lung cancer, breast cancer, bladder cancer, pancreatic cancer, ovarian cancer and prostate cancer.

Suitable routes of administering the antibody compositions (e.g., human monoclonal antibodies, multispecific and bispecific molecules and immuno conjugates) of the invention in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, human anti-PTK7 antibodies of the invention can be coadministered with one or other more therapeutic agents, e.g., a cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the human anti-PTK7 antibodies or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

When administering antibody-partner molecule conjugates of the present invention for use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmmol/kg/day or less. For example, the patient doses can be 0.9, 0.6, 0.5, 0.45, 0.3, 0.2, 0.15, or 0.1 μmmol/kg/day or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

In one embodiment, immunoconjugates of the invention can be used to target compounds (e.g., therapeutic agents, labels, cytotoxins, radiotoxoins immunosuppressants, etc.) to cells which have PTK7 cell surface receptors by linking such compounds to the antibody. For example, an anti-PTK7 antibody can be conjugated to any of the toxin compounds described in U.S. Pat. Nos. 6,281,354 and 6,548,530, US patent publication Nos. 20030050331, 20030064984, 20030073852 and 20040087497 or published in WO 03/022806, which are hereby incorporated by reference in their entireties. Thus, the invention also provides methods for localizing ex vivo or in vivo cells expressing PTK7 (e.g., with a detectable label, such as a radioisotope, a fluorescent compound, an enzyme or an enzyme co-factor). Alternatively, the immunoconjugates can be used to kill cells which have PTK7 cell surface receptors by targeting cytotoxins or radiotoxins to PTK7.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing PTK7 and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions can be used in conjunction with chemotherapy. Additionally, combination immunotherapy may be used to direct two distinct cytotoxic effector populations toward tumor cell rejection. For example, anti-PTK7 antibodies linked to anti-Fc-gamma RI or anti-CD3 may be used in conjunction with IgG- or IgA-receptor specific binding agents.

Bispecific and multispecific molecules of the invention can also be used to modulate FcγR or FcγR levels on effector cells, such as by capping and elimination of receptors on the cell surface. Mixtures of anti-Fc receptors can also be used for this purpose.

The compositions (e.g., human antibodies, multispecific and bispecific molecules and immunoconjugates) of the invention which have complement binding sites, such as portions from IgG1, -2 or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions (e.g., human antibodies, multispecific and bispecific molecules and immunoconjugates) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Accordingly, patients treated with antibody compositions of the invention can be additionally administered (prior to, simultaneously with or following administration of a human antibody of the invention) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ) and tumor necrosis factor (TNF).

The compositions (e.g., human antibodies, multispecific and bispecific molecules) of the invention can also be used to target cells expressing FcγR or PTK7, for example for labeling such cells. For such use, the binding agent can be linked to a molecule that can be detected. Thus, the invention provides methods for localizing ex vivo or in vitro cells expressing Fc receptors, such as FcγR or PTK7. The detectable label can be, e.g., a radioisotope, a fluorescent compound, an enzyme or an enzyme co-factor.

Also within the scope of the present invention are kits comprising the antibody compositions of the invention (e.g., human antibodies, bispecific or multispecific molecules, or immunoconjugates) and instructions for use. The kit can further contain one more additional reagents, such as an immunosuppressive reagent, a cytotoxic agent or a radiotoxic agent or one or more additional human antibodies of the invention (e.g., a human antibody having a complementary activity which binds to an epitope in the PTK7 antigen distinct from the first human antibody). Kits typically include a label indicating the intended use of the contents of the kit. The term label includes any writing, or recorded material supplied on or with the kit, or which otherwise accompanies the kit.

The present invention is further illustrated by the following examples, which should not be construed as further limiting. The contents of all figures and all references, patents and published patent applications cited throughout this application are expressly incorporated herein by reference in their entirety.

### EXAMPLES

### Example 1

### Generation of Human Monoclonal Antibodies Against PTK7

### Antigen

Immunization protocols utilized as antigen both (i) a recombinant fusion protein comprising the extracellular portion of PTK7 with both a myc and his tag and (ii) membrane bound full-length PTK7. Both antigens were generated by recombinant transfection methods in a CHO cell line.

### Transgenic HuMab and KM Mice™

Fully human monoclonal antibodies to PTK7 were prepared using the HCo7 and HCo12 strains of HuMab transgenic mice and the KM strain of transgenic transchromosomic mice, each of which express human antibody genes. In each of these mouse strains, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Each of these mouse strains carries a human kappa light chain transgene, KCo5, as described in Fishwild et al. (1996) Nature Biotechnology 14:845-851. The HCo7 strain carries the HCo7 human heavy chain transgene as described in U.S. Pat. Nos. 5,770,429; 5,545,806; 5,625,825; and 5,545,807. The HCo12 strain carries the HCo12 human heavy chain transgene as described in Example 2 of WO 01/09187 or example 2 WO 01/14424. The KM strain contains the SC20 transchromosome as described in PCT Publication WO 02/43478.

### HuMab and KM Immunizations:

To generate fully human monoclonal antibodies to PTK7, HuMab mice and KM Mice™ were immunized with purified recombinant PTK7 fusion protein and PTK7-transfected CHO cells as antigen. General immunization schemes for HuMab mice are described in Lonberg, N. et al (1994) Nature 368(6474): 856-859; Fishwild, D. et al. (1996) Nature Biotechnology 14: 845-851 and PCT Publication WO 98/24884. The mice were 6-16 weeks of age upon the first infusion of antigen. A purified recombinant preparation (5-50 μg) of PTK7 fusion protein antigen and 5-10×10⁶ cells were used to immunize the HuMab mice and KM Mice™ intraperitonealy, subcutaneously (Sc) or via footpad injection.

Transgenic mice were immunized twice with antigen in complete Freund's adjuvant or Ribi adjuvant IP, followed by 3-21 days IP (up to a total of 11 immunizations) with the antigen in incomplete Freund's or Ribi adjuvant. The immune response was monitored by retroorbital bleeds. The plasma was screened by ELISA (as described below), and mice with sufficient titers of anti-PTK7 human immunoglobulin were used for fusions. Mice were boosted intravenously with antigen 3 days before sacrifice and removal of the spleen. Typically, 10-35 fusions for each antigen were performed. Several dozen mice were immunized for each antigen.

### Selection of HuMab or KM Mice™ Producing Anti-PTK7 Antibodies:

To select HuMab or KM Mice™ producing antibodies that bound PTK7, sera from immunized mice were tested by ELISA as described by Fishwild, D. et al. (1996). Briefly, microtiter plates were coated with purified recombinant PTK7 fusion protein from transfected CHO cells at 1-2 μg/ml in PBS, 100 μl/wells incubated 4° C. overnight then blocked with 200 μl/well of 5% fetal bovine serum in PBS/Tween (0.05%). Dilutions of sera from PTK7-immunized mice were added to each well and incubated for 1-2 hours at ambient temperature. The plates were washed with PBS/Tween and then incubated with a goat-anti-human IgG polyclonal antibody conjugated with horseradish peroxidase (HRP) for 1 hour at room temperature. After washing, the plates were developed with ABTS substrate (Sigma, A-1888, 0.22 mg/ml) and analyzed by spectrophotometer at OD 415-495. Mice that developed the highest titers of anti-PTK7 antibodies were used for fusions. Fusions were performed as described below and hybridoma supernatants were tested for anti-PTK7 activity by ELISA.

### Generation of Hybridomas Producing Human Monoclonal Antibodies to PTK7:

The mouse splenocytes, isolated from the HuMab mice, were fused with PEG to a mouse myeloma cell line based upon standard protocols. The resulting hybridomas were then screened for the production of antigen-specific antibodies. Single cell suspensions of splenocytes from immunized mice were fused to one-fourth the number of SP2/0 nonsecreting mouse myeloma cells (ATCC, CRL 1581) with 50% PEG (Sigma). Cells were plated at approximately 1×10⁵/well in flat bottom microtiter plate, followed by about two week incubation in selective medium containing 10% fetal bovine serum, 10% P388D1 (ATCC, CRL TIB-63) conditioned medium, 3-5% origen (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After 1-2 weeks, cells were cultured in medium in which the HAT was replaced with HT. Individual wells were then screened by ELISA (described above) for human anti-PTK7 monoclonal IgG antibodies. Once extensive hybridoma growth occurred, medium was monitored usually after 10-14 days. The antibody-secreting hybridomas were replated, screened again and, if still positive for human IgG, anti-PTK7 monoclonal antibodies were subcloned at least twice by limiting dilution. The stable subclones were then cultured in vitro to generate small amounts of antibody in tissue culture medium for further characterization.

Hybridoma clones 3G8, 3G8a, 4D5, 12C6, 12C6a and 7C8 were selected for further analysis.

### Example 2

### Structural Characterization of Human Monoclonal Antibodies 3G8, 3G8a, 4D5, 12C6, 12C6a and 7C8

The cDNA sequences encoding the heavy and light chain variable regions of the 3G8, 3G8a, 4D5, 12C6, 12C6a and 7C8 monoclonal antibodies were obtained from the 3G8, 3G8a, 4D5, 12C6, 12C6a and 7C8 hybridomas, respectively, using standard PCR techniques and were sequenced using standard DNA sequencing techniques.

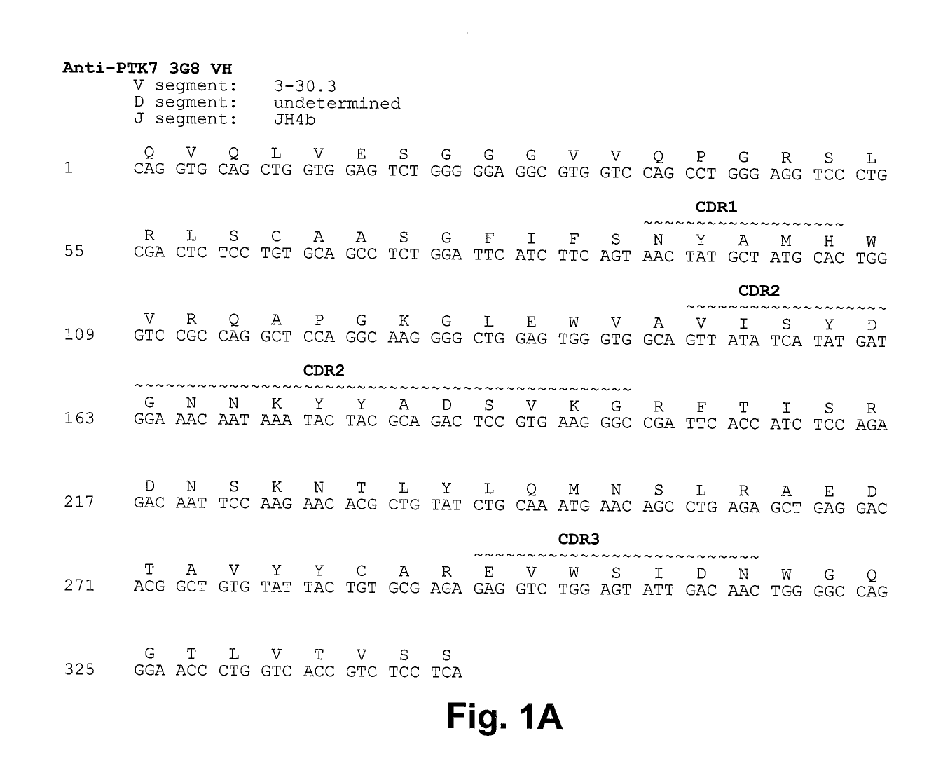

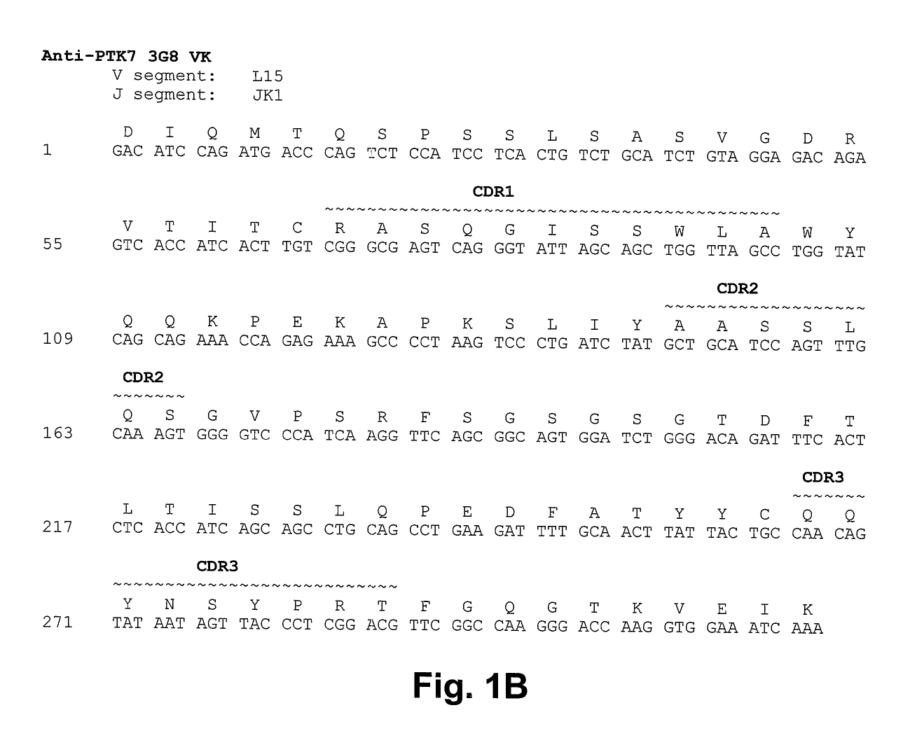

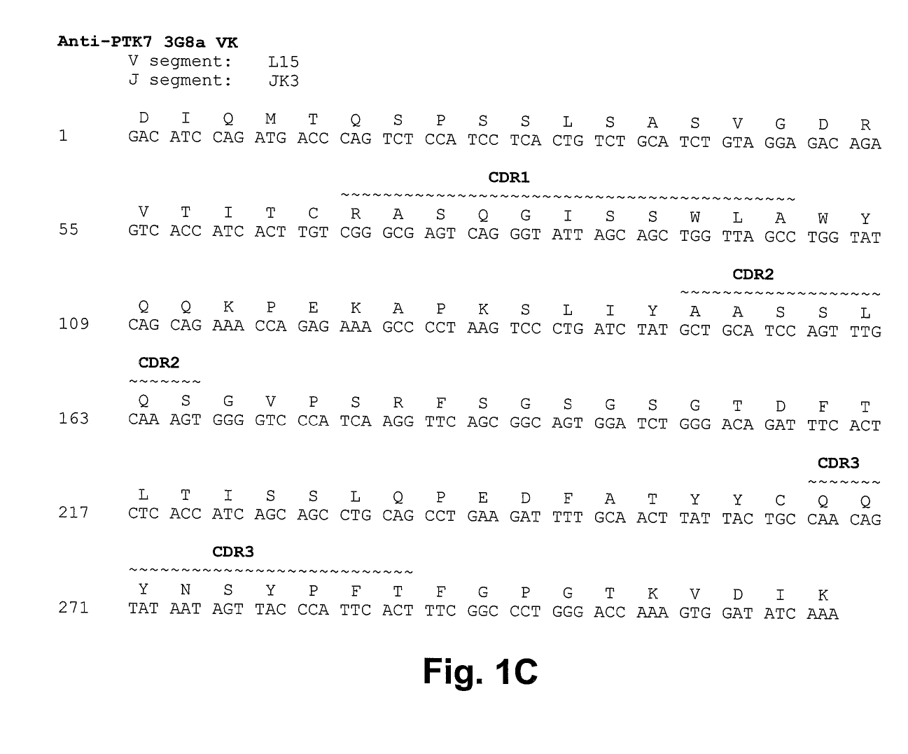

The nucleotide and amino acid sequences of the heavy chain variable region of 3G8 are shown in FIG. 1A and in SEQ ID NO:41 and 1, respectively.

The nucleotide and amino acid sequences of the light chain variable region of 3G8 are shown in FIG. 1B and in SEQ ID NO:45 and 5, respectively.

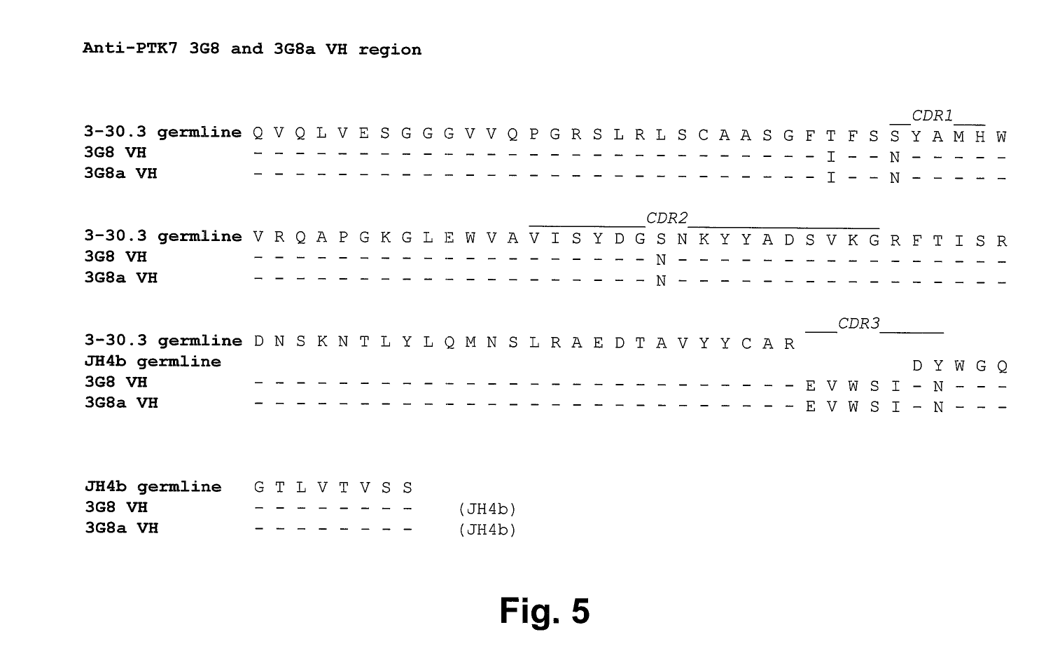

Comparison of the 3G8 heavy chain immunoglobulin sequence to the known human germline immunoglobulin heavy chain sequences demonstrated that the 3G8 heavy chain utilizes a VH segment from human germline VH 3-30.3, an undetermined D segment, and a JH segment from human germline JH 4b. The alignment of the 3G8 VH sequence to the germline VH 3-30.3 sequence is shown in FIG. 5. Further analysis of the 3G8 VH sequence using the Kabat system of CDR region determination led to the delineation of the heavy chain CDR1, CDR2 and CD3 regions as shown in FIGS. 1A and 5, and in SEQ ID NOs: 11, 15 and 19, respectively.

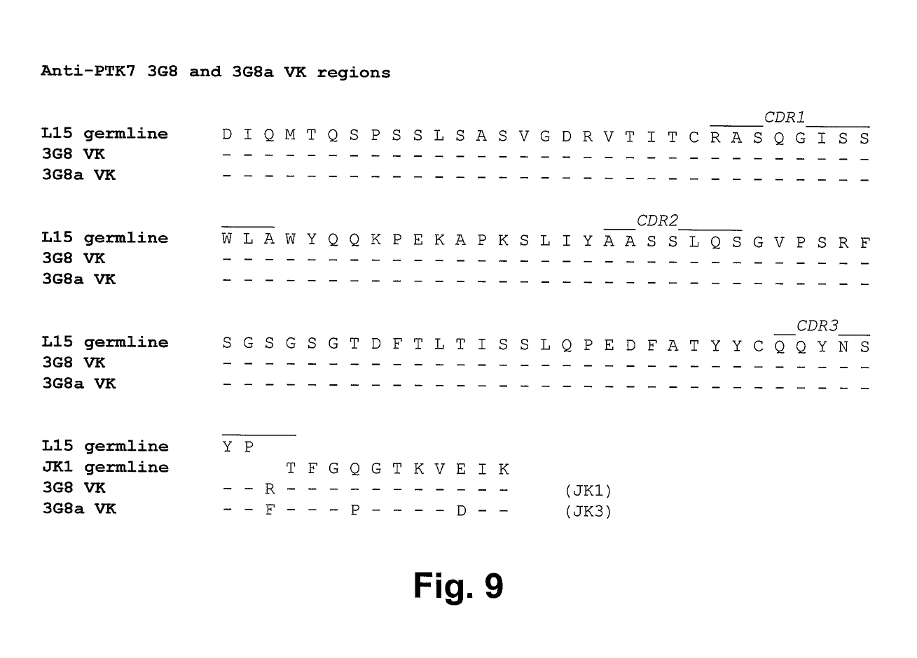

Comparison of the 3G8 light chain immunoglobulin sequence to the known human germline immunoglobulin light chain sequences demonstrated that the 3G8 light chain utilizes a VL segment from human germline VK L15 and a JK segment from human germline JK 1. The alignment of the 3G8 VL sequence to the germline VK L15 sequence is shown in FIG. 9. Further analysis of the 3G8 VL sequence using the Kabat system of CDR region determination led to the delineation of the light chain CDR1, CDR2 and CD3 regions as shown in FIGS. 1B and 9, and in SEQ ID NOs: 23, 29 and 35, respectively.

The nucleotide and amino acid sequences of the heavy chain variable region of 3G8a are shown in FIG. 1A and in SEQ ID NO:41 and 1, respectively.

The nucleotide and amino acid sequences of the light chain variable region of 3G8a are shown in FIG. 1C and in SEQ ID NO:46 and 6, respectively.

Comparison of the 3G8a heavy chain immunoglobulin sequence to the known human germline immunoglobulin heavy chain sequences demonstrated that the 3G8a heavy chain utilizes a VH segment from human germline VH 3-30.3, an undetermined D segment, and a JH segment from human germline JH 4b. The alignment of the 3G8a VH sequence to the germline VH 3-30.3 sequence is shown in FIG. 5. Further analysis of the 3G8a VH sequence using the Kabat system of CDR region determination led to the delineation of the heavy chain CDR1, CDR2 and CD3 regions as shown in FIGS. 1A and 5, and in SEQ ID NOs: 11, 15 and 19, respectively.

Comparison of the 3G8a light chain immunoglobulin sequence to the known human germline immunoglobulin light chain sequences demonstrated that the 3G8a light chain utilizes a VL segment from human germline VK L15 and a JK segment from human germline JK 3. The alignment of the 3G8a VL sequence to the germline VK L15 sequence is shown in FIG. 9. Further analysis of the 3G8a VL sequence using the Kabat system of CDR region determination led to the delineation of the light chain CDR1, CDR2 and CD3 regions as shown in FIGS. 1C and 9, and in SEQ ID NOs: 24, 30 and 36, respectively.

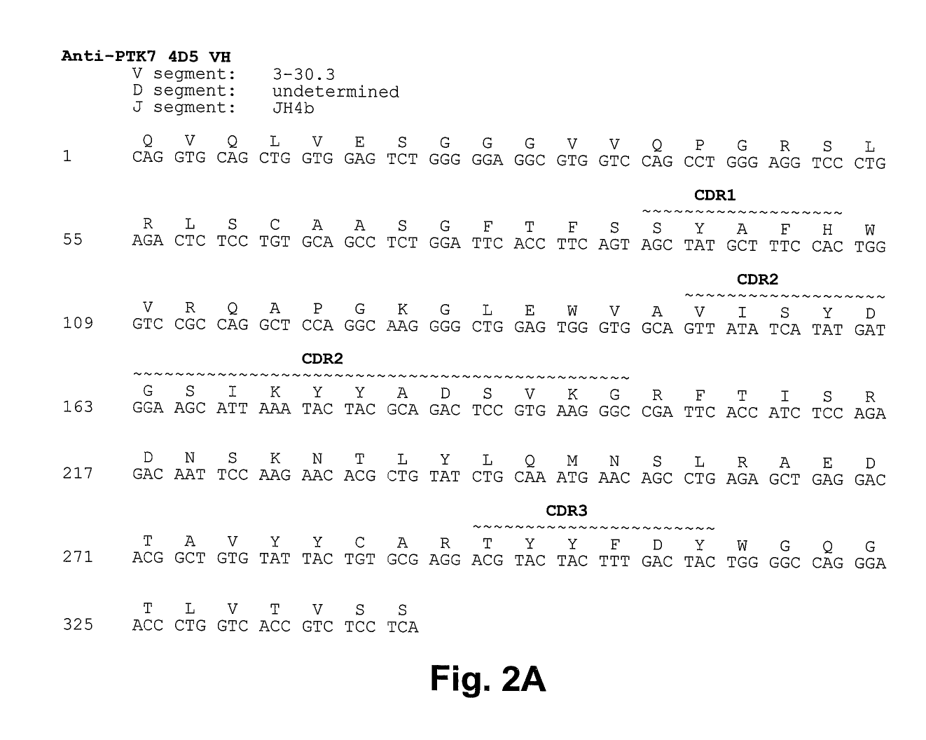

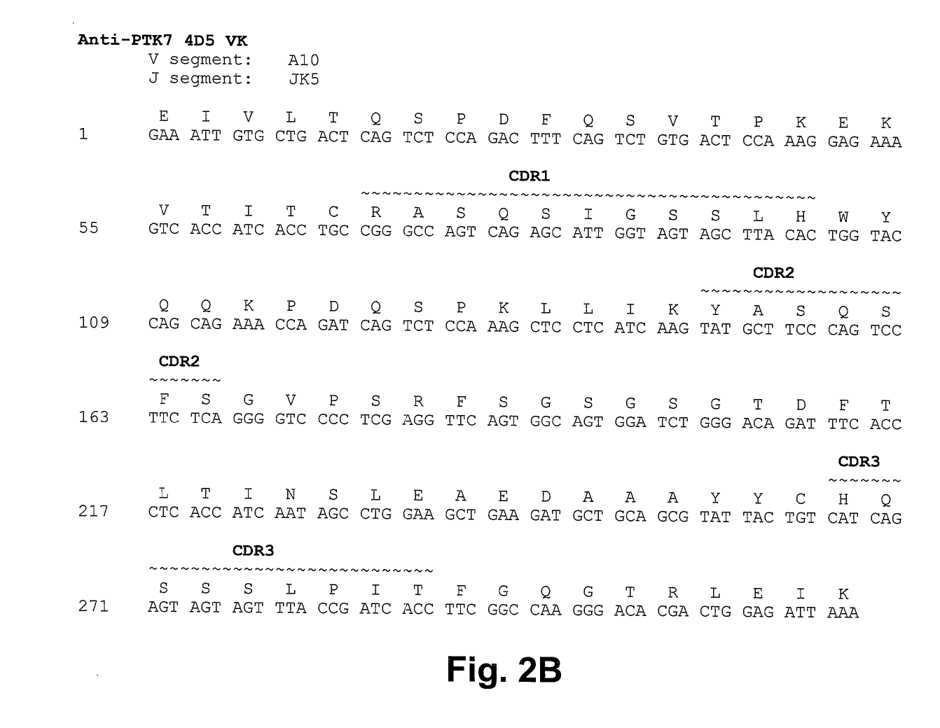

The nucleotide and amino acid sequences of the heavy chain variable region of 4D5 are shown in FIG. 2A and in SEQ ID NO:42 and 2, respectively.

The nucleotide and amino acid sequences of the light chain variable region of 4D5 are shown in FIG. 2B and in SEQ ID NO:47 and 7, respectively.

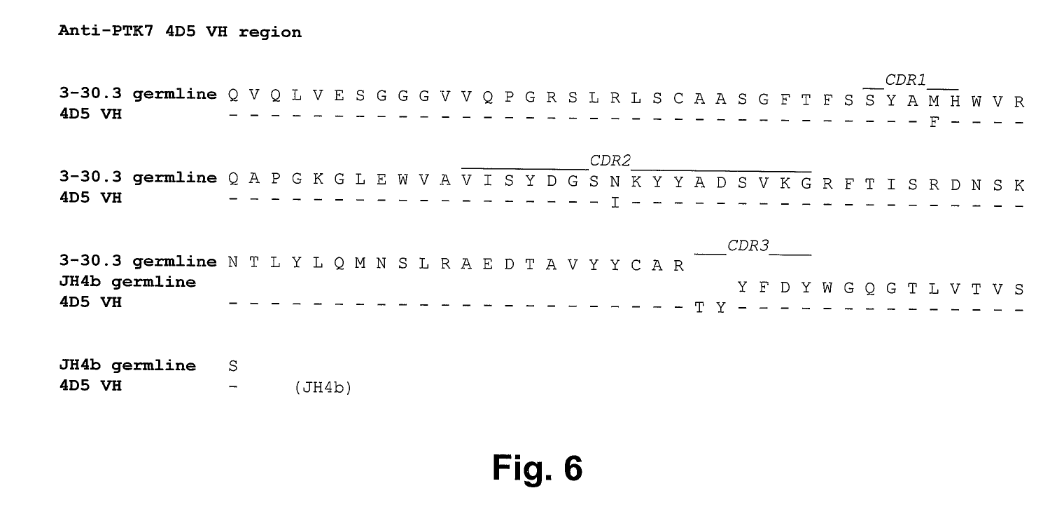

Comparison of the 4D5 heavy chain immunoglobulin sequence to the known human germline immunoglobulin heavy chain sequences demonstrated that the 4D5 heavy chain utilizes a VH segment from human germline VH 3-30.3, an undetermined D segment, and a JH segment from human germline JH 4b. The alignment of the 4D5 VH sequence to the germline VH 3-30.3 sequence is shown in FIG. 6. Further analysis of the 4D5 VH sequence using the Kabat system of CDR region determination led to the delineation of the heavy chain CDR1, CDR2 and CD3 regions as shown in FIGS. 2A and 6, and in SEQ ID NOs: 12, 16 and 20, respectively.

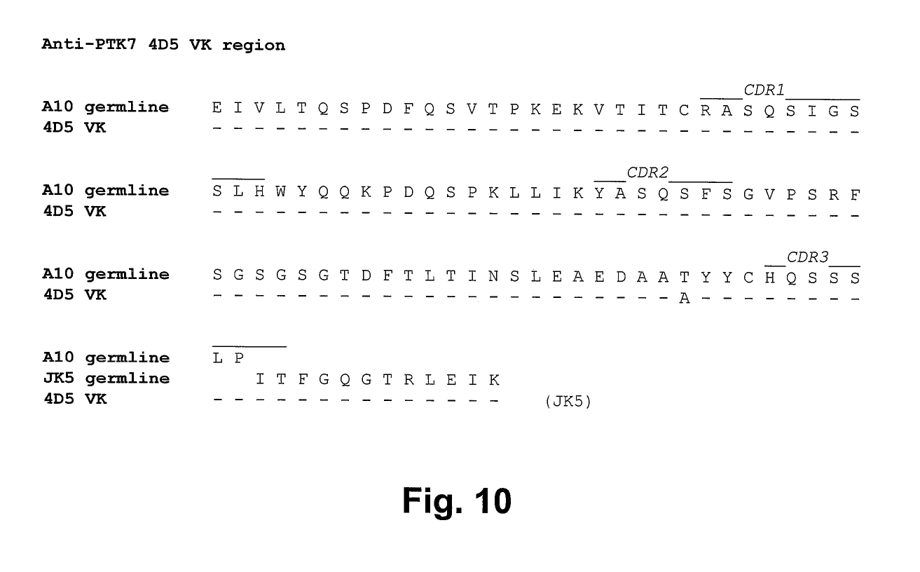

Comparison of the 4D5 light chain immunoglobulin sequence to the known human germline immunoglobulin light chain sequences demonstrated that the 4D5 light chain utilizes a VL segment from human germline VK A10 and a JK segment from human germline JK 5. The alignment of the 4D5 VL sequence to the germline VK A10 sequence is shown in FIG. 10. Further analysis of the 4D5 VL sequence using the Kabat system of CDR region determination led to the delineation of the light chain CDR1, CDR2 and CD3 regions as shown in FIGS. 2B and 10, and in SEQ ID NOs: 25, 31 and 37, respectively.

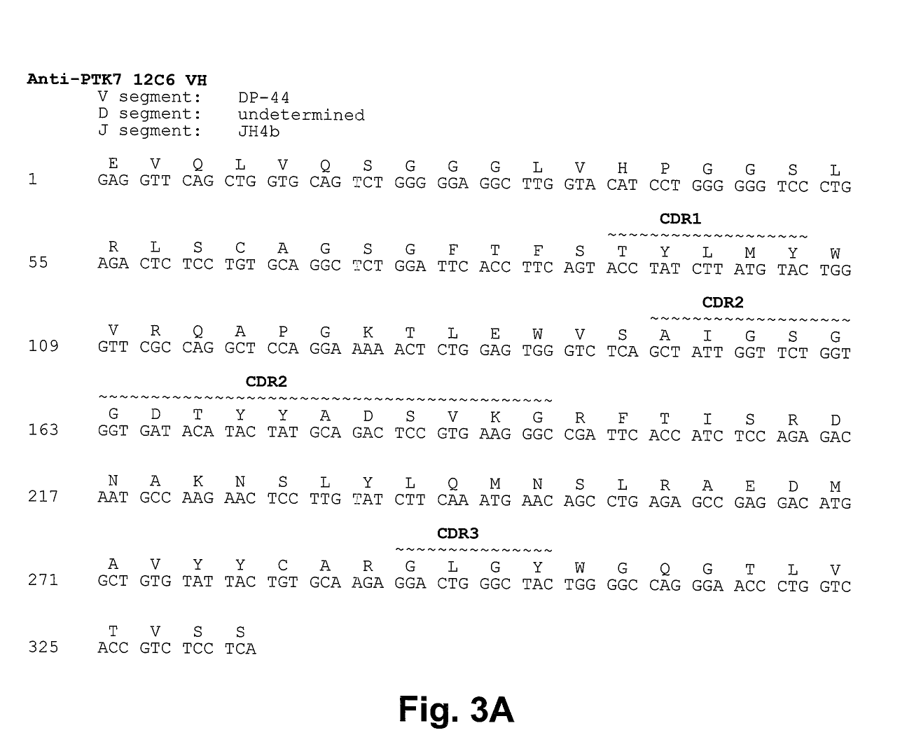

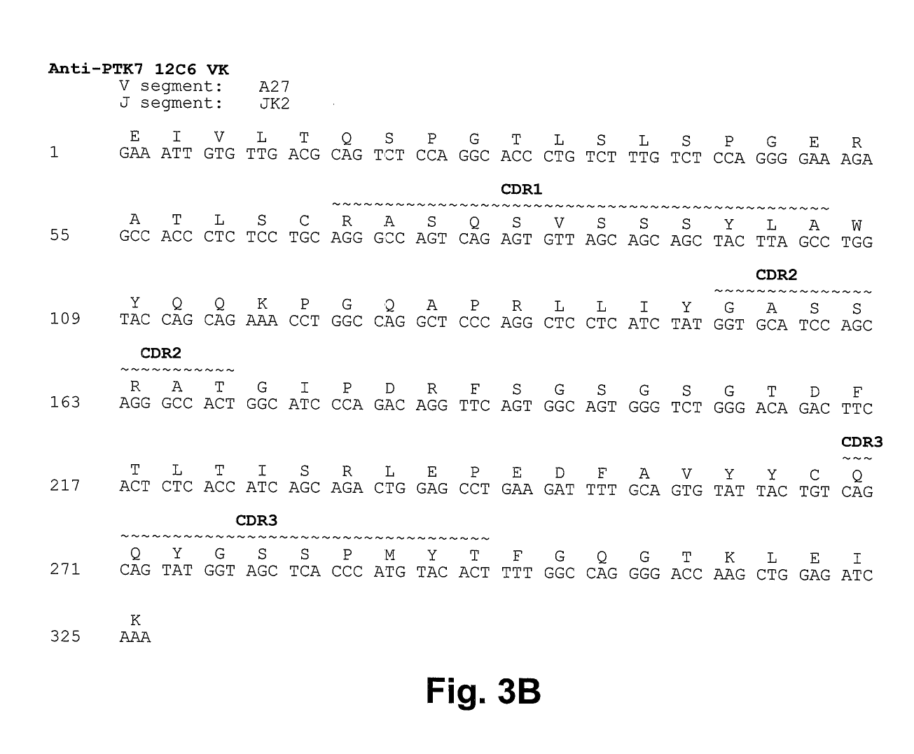

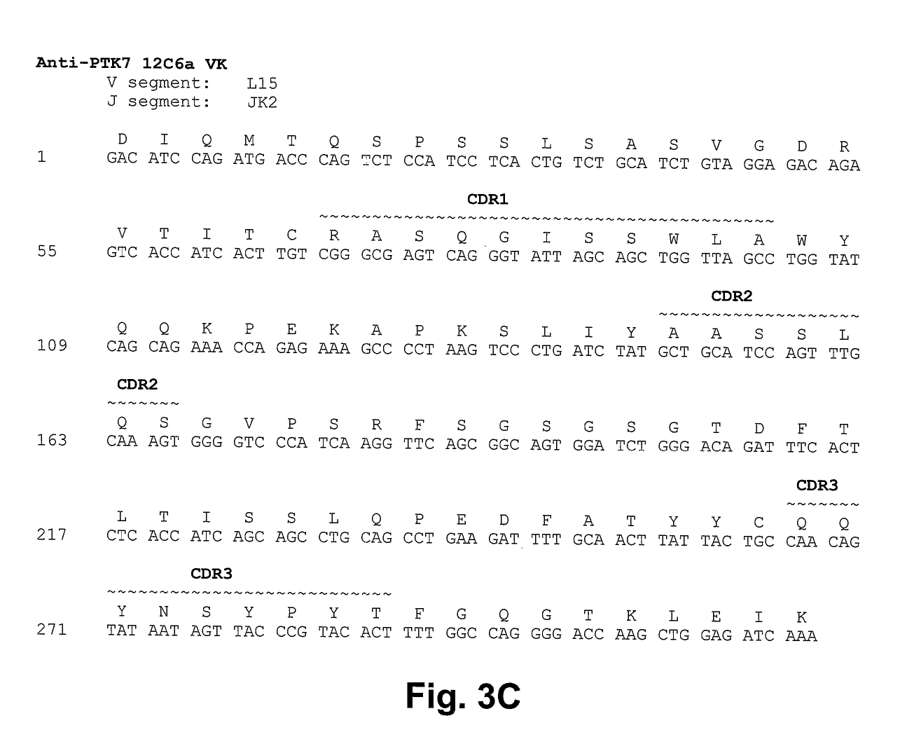

The nucleotide and amino acid sequences of the heavy chain variable region of 12C6 are shown in FIG. 3A and in SEQ ID NO:43 and 3, respectively.

The nucleotide and amino acid sequences of the light chain variable region of 12C6 are shown in FIG. 3B and in SEQ ID NO:48 and 8, respectively.

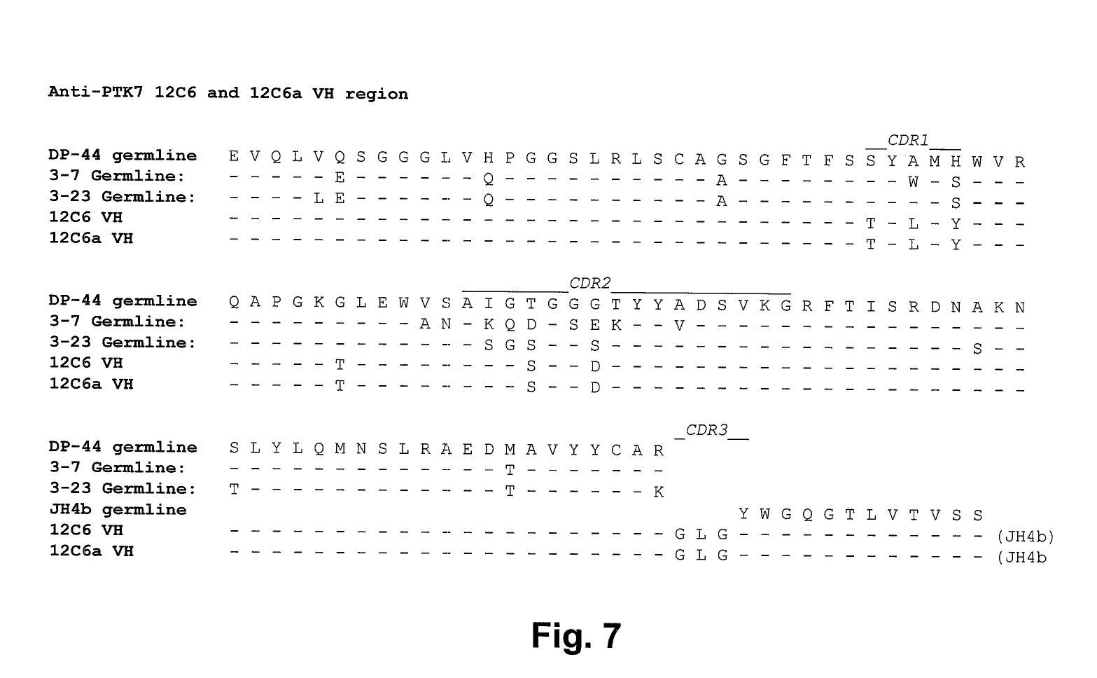

Comparison of the 12C6 heavy chain immunoglobulin sequence to the known human germline immunoglobulin heavy chain sequences demonstrated that the 12C6 heavy chain utilizes a VH segment from human germline VH DP44, an undetermined D segment, and a JH segment from human germline JH 4b. The alignment of the 12C6 VH sequence to the germline VH DP44 sequence is shown in FIG. 7. Further analysis of the 12C6 VH sequence using the Kabat system of CDR region determination led to the delineation of the heavy chain CDR1, CDR2 and CD3 regions as shown in FIGS. 3A and 7, and in SEQ ID NOs: 13, 17 and 21, respectively.

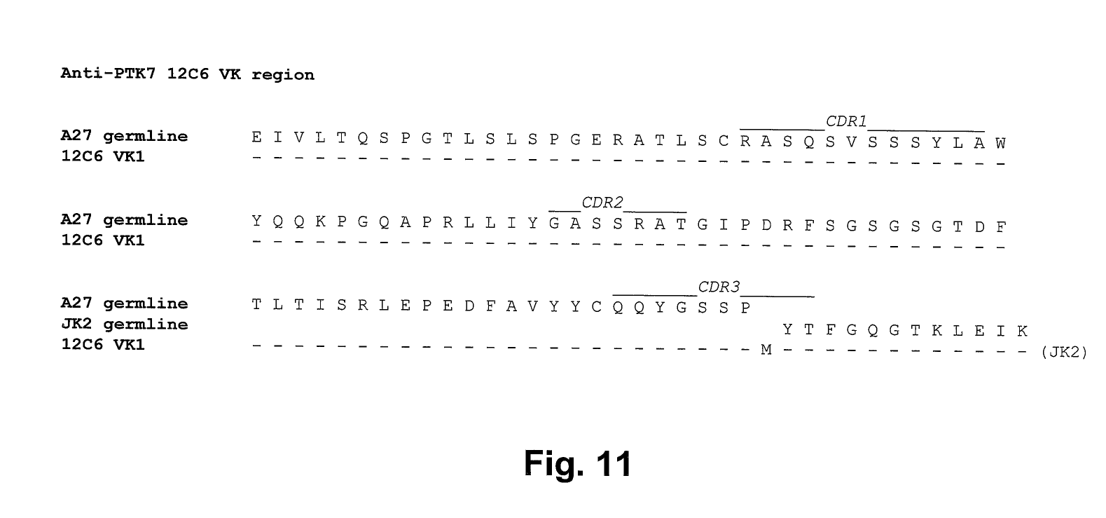

Comparison of the 12C6 light chain immunoglobulin sequence to the known human germline immunoglobulin light chain sequences demonstrated that the 12C6 light chain utilizes a VL segment from human germline VK A27 and a JK segment from human germline JK 2. The alignment of the 12C6 VL sequence to the germline VK A27 sequence is shown in FIG. 11. Further analysis of the 12C6 VL sequence using the Kabat system of CDR region determination led to the delineation of the light chain CDR1, CDR2 and CD3 regions as shown in FIGS. 3B and 11, and in SEQ ID NOs: 26, 32 and 38, respectively.

The nucleotide and amino acid sequences of the heavy chain variable region of 12C6a are shown in FIG. 3A and in SEQ ID NO:43 and 3, respectively.

The nucleotide and amino acid sequences of the light chain variable region of 12C6a are shown in FIG. 3C and in SEQ ID NO:49 and 9, respectively.

Comparison of the 12C6a heavy chain immunoglobulin sequence to the known human germline immunoglobulin heavy chain sequences demonstrated that the 12C6a heavy chain utilizes a VH segment from human germline VH DP44, an undetermined D segment, and a JH segment from human germline JH 4b. The alignment of the 12C6a VH sequence to the germline VH DP44 sequence is shown in FIG. 7. Further analysis of the 12C6a VH sequence using the Kabat system of CDR region determination led to the delineation of the heavy chain CDR1, CDR2 and CD3 regions as shown in FIGS. 3A and 7, and in SEQ ID NOs: 13, 17 and 21, respectively.

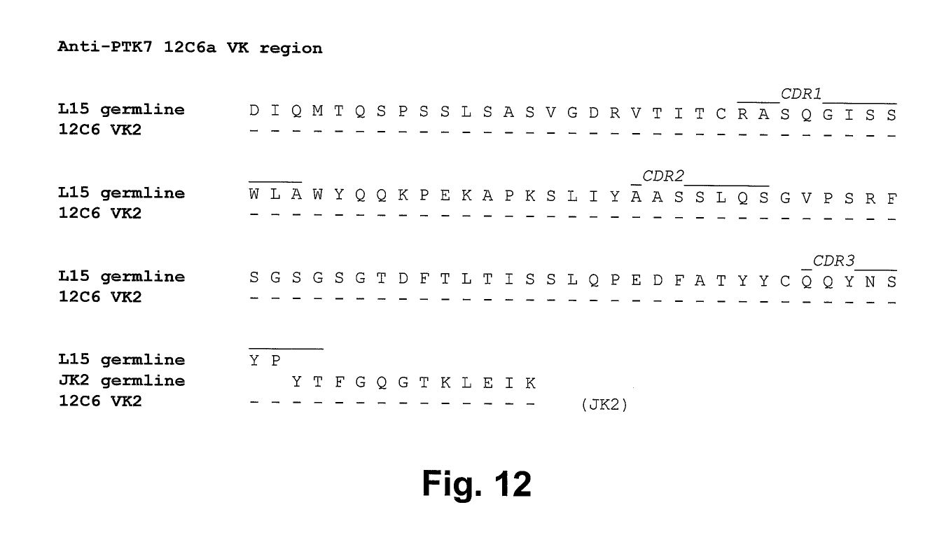

Comparison of the 12C6a light chain immunoglobulin sequence to the known human germline immunoglobulin light chain sequences demonstrated that the 12C6a light chain utilizes a VL segment from human germline VK L15 and a JK segment from human germline JK 2. The alignment of the 12C6a VL sequence to the germline VK L15 sequence is shown in FIG. 12. Further analysis of the 12C6a VL sequence using the Kabat system of CDR region determination led to the delineation of the light chain CDR1, CDR2 and CD3 regions as shown in FIGS. 3C and 12, and in SEQ ID NOs: 27, 33 and 39, respectively.

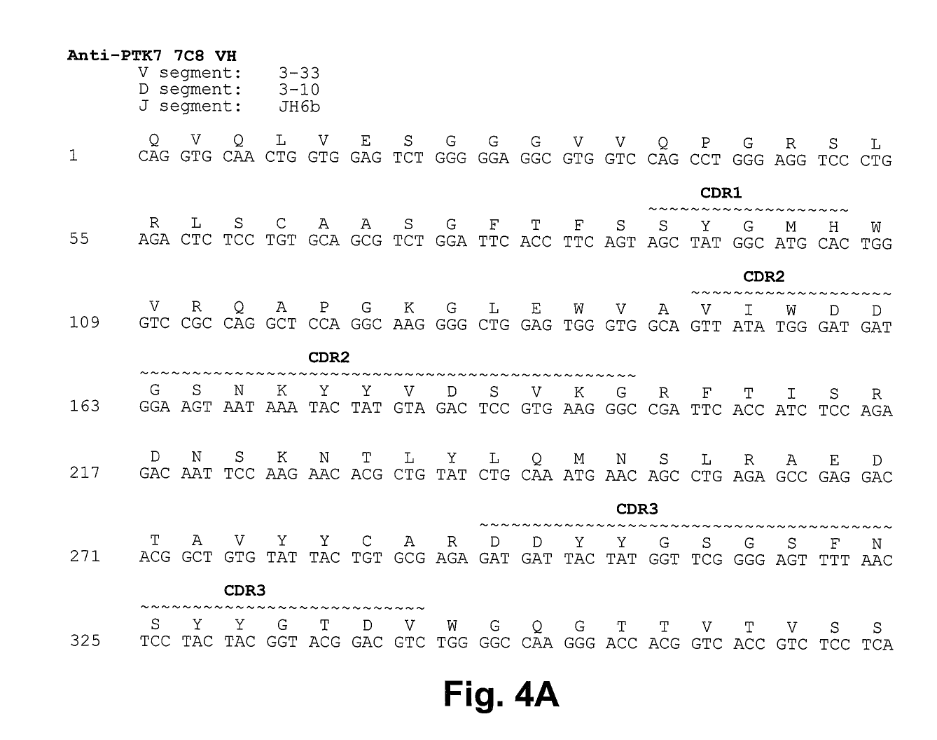

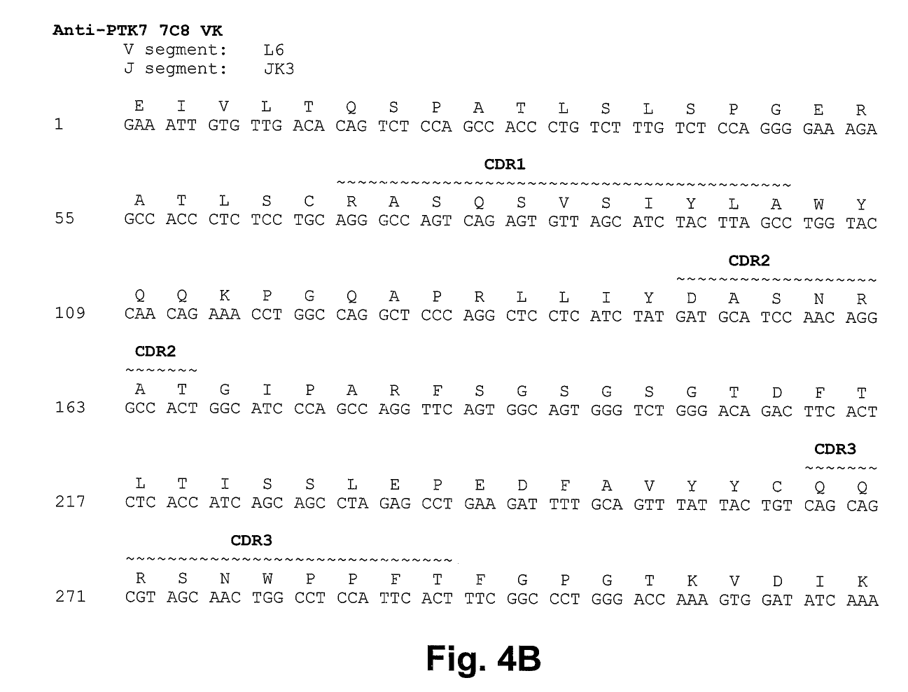

The nucleotide and amino acid sequences of the heavy chain variable region of 7C8 are shown in FIG. 4A and in SEQ ID NO:44 and 4, respectively.

The nucleotide and amino acid sequences of the light chain variable region of 7C8 are shown in FIG. 4B and in SEQ ID NO:50 and 10, respectively.

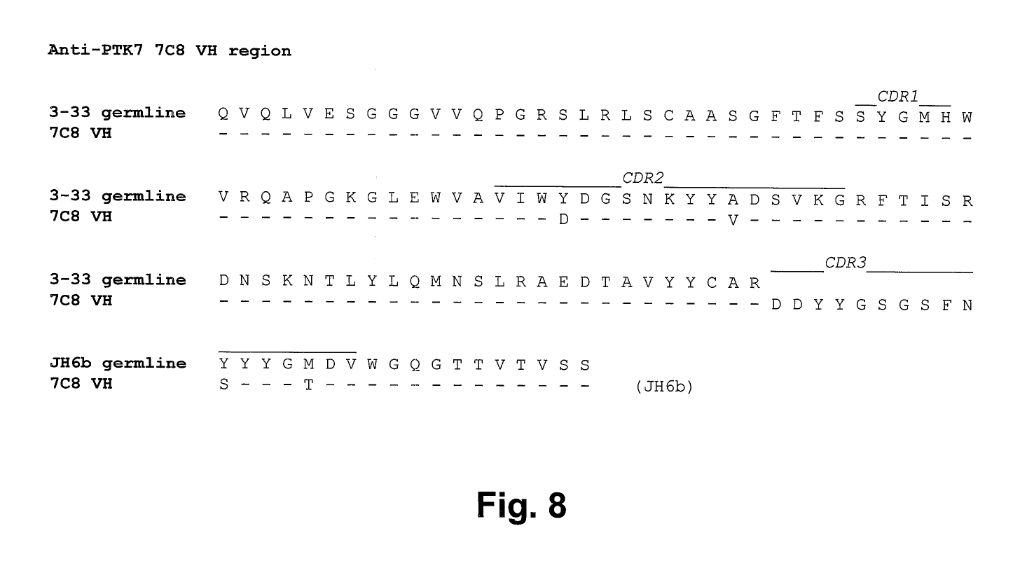

Comparison of the 7C8 heavy chain immunoglobulin sequence to the known human germline immunoglobulin heavy chain sequences demonstrated that the 7C8 heavy chain utilizes a VH segment from human germline VH 3-33, a D segment from human germline 3-10, and a JH segment from human germline JH 6b. The alignment of the 7C8 VH sequence to the germline VH 3-33 sequence is shown in FIG. 8. Further analysis of the 7C8 VH sequence using the Kabat system of CDR region determination led to the delineation of the heavy chain CDR1, CDR2 and CD3 regions as shown in FIGS. 4A and 8, and in SEQ ID NOs: 14, 18 and 22, respectively.

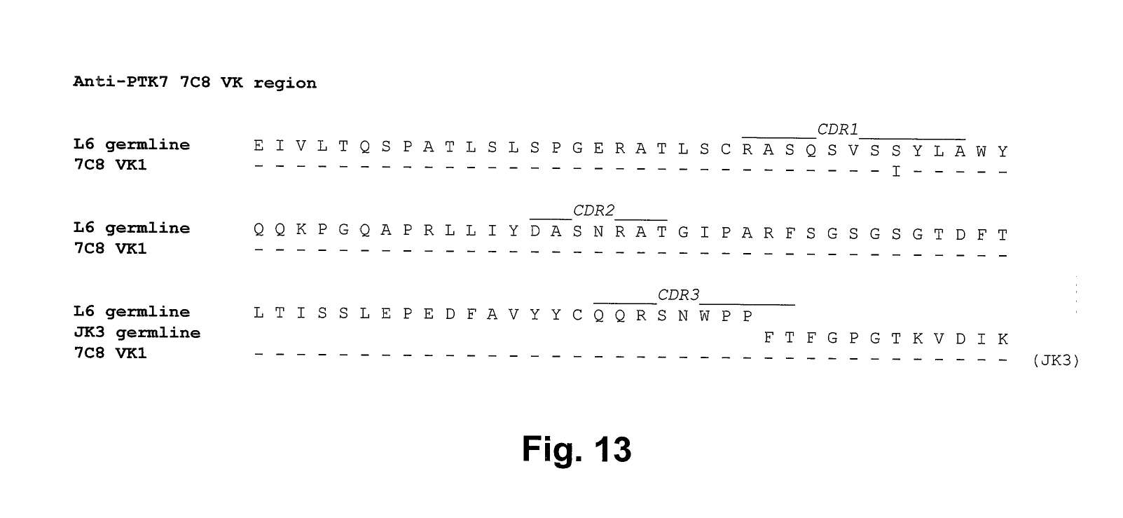

Comparison of the 7C8 light chain immunoglobulin sequence to the known human germline immunoglobulin light chain sequences demonstrated that the 7C8 light chain utilizes a VL segment from human germline VK L6 and a JK segment from human germline JK 3. The alignment of the 7C8 VL sequence to the germline VK L6 sequence is shown in FIG. 13. Further analysis of the 7C8 VL sequence using the Kabat system of CDR region determination led to the delineation of the light chain CDR1, CDR2 and CD3 regions as shown in FIGS. 4B and 13, and in SEQ ID NOs: 28, 34 and 40, respectively.

### Example 3

### Mutation of mAb 12C6 and Alternative Germline Usage

As discussed in Example 2 above, monoclonal antibodies 12C6 and 12C6a utilize a heavy chain variable region derived from a human DP-44 germline sequence present in the HCo7 transgene of the HuMab Mouse® strain. Since DP-44 is not a germline sequence that is utilized in the native human immunoglobulin repertoire, it may be advantageous to mutate the VH sequence of 12C6 and 12C6a to reduce potential immunogenicity. Preferably, one or more framework residues of the 12C6 or 12C6a VH sequence is mutated to a residue(s) present in the framework of a structurally related VH germline sequence that is utilized in the native human immunoglobulin repertoire. For example, FIG. 7 shows the alignment of the 12C6 and 12C6a VH sequence with the DP44 germline sequence and also to two structurally related human germline sequences, VH 3-23 and VH 3-7. Given the relatedness of these sequences, one can predict that one can select a human antibody that specifically binds to human PTK7 and that utilizes a VH region derived from a VH 3-23 or VH 3-7 germline sequence. Moreover, one can mutate one or more residues within the 12C6 or 12C6a VH sequence that differ from the residue(s) at the comparable position in the VH 3-23 or VH 3-7 sequence to the residue(s) that is present in VH 3-23 or VH 3-7, or to a conservative amino acid substitution thereof.

### Example 4

### Characterization of Binding Specificity and Binding Kinetics of Anti-PTK7 Human Monoclonal Antibodies

In this example, binding affinity and binding kinetics of anti-PTK7 antibodies were examined by Biacore analysis. Binding specificity, and cross-competition were examined by flow cytometry.

### Binding Specificity by Flow Cytometry

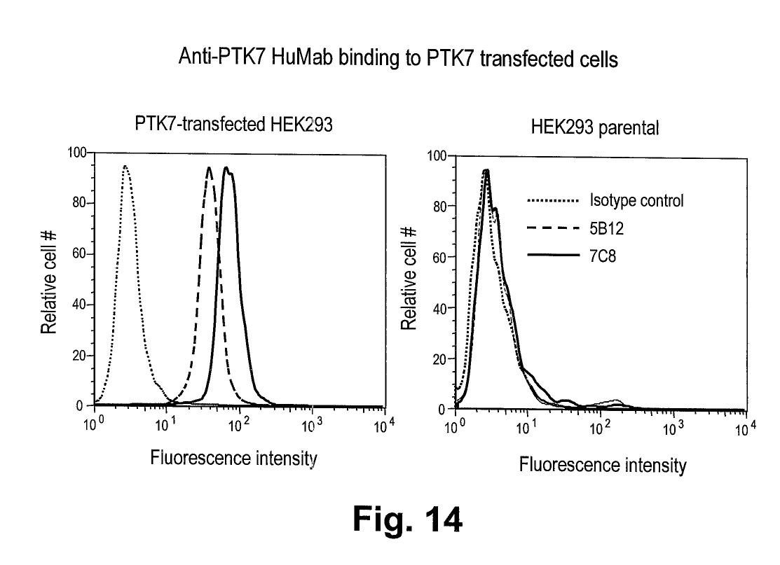

HEK3 cell lines that express recombinant human PTK7 at the cell surface were developed and used to determine the specificity of PTK7 human monoclonal antibodies by flow cytometry. HEK3 cells were transfected with expression plasmids containing full length cDNA encoding transmembrane forms of PTK7. Binding of the 7C8 anti-PTK7 human monoclonal antibody was assessed by incubating the transfected cells with the anti-PTK7 human monoclonal antibody at a concentration of 10 μg/ml. The cells were washed and binding was detected with a FITC-labeled anti-human IgG Ab. Flow cytometric analyses were performed using a FACScan flow cytometry (Becton Dickinson, San Jose, Calif.). The results are depicted in FIG. 14. The anti-PTK7 human monoclonal antibody 7C8 bound to the HEK3 cells transfected with PTK7 but not to HEK3 cells that were not transfected with human PTK7. This data demonstrates the specificity of anti-PTK7 human monoclonal antibodies for PTK7.

### Binding Specificity by ELISA

The binding of anti-PTK7 antibodies was also assessed by standard ELISA to examine the specificity of binding for PTK7.

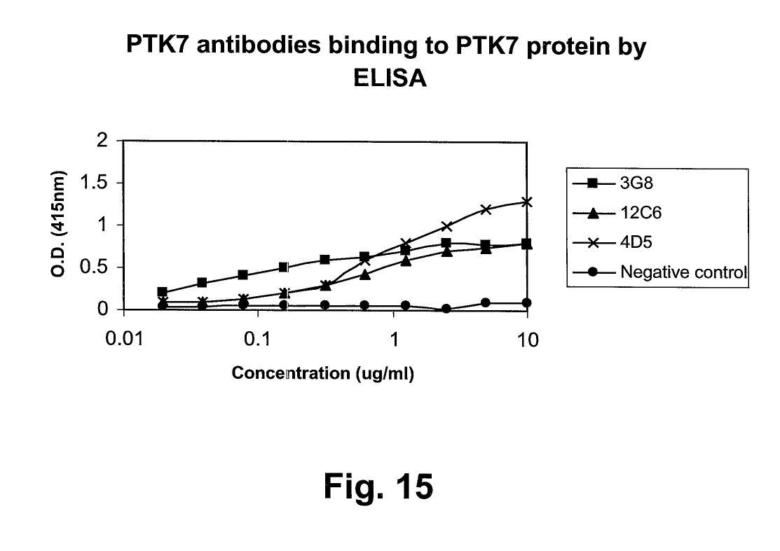

Recombinant extracellular domain of PTK7 was tested for binding against the anti-PTK7 human monoclonal antibodies 3G8, 4D5, 12C6 and 12C6a at different concentrations. Standard ELISA procedures were performed. The anti-PTK7 human monoclonal antibodies were added at a starting concentration of 10 μg/ml and serially diluted at a 1:2 dilution. Goat-anti-human IgG (kappa chain-specific) polyclonal antibody conjugated with horseradish peroxidase (HRP) was used as secondary antibody. The results are shown in FIG. 15. Each of the anti-PTK7 human monoclonal antibodies 3G8, 4D5, 12C6 and 12C6a bound to PTK7. This data demonstrates the specificity of anti-PTK7 human monoclonal antibodies for PTK7.

### Epitope Mapping of Anti-PTK7 Antibodies

Flow cytometry was used to determine epitope grouping of anti-PTK7 HuMAbs. Wilms' tumor cells G-401 (ATCC Acc No. CRL-1441) were transfected with expression plasmids containing full length cDNA encoding transmembrane forms of PTK7. Epitope binding of each anti-PTK7 human monoclonal antibody was assessed by incubating 1×10⁵ transfected cells with 10 μg/ml of cold anti-PTK7 human monoclonal antibody, washed, and followed by the addition of 10 μg/ml of a fluorescence-conjugated anti-PTK7 human monoclonal antibody. Binding was detected with a FITC-labeled anti-human IgG Ab. Flow cytometric analyses were performed using a FACScan flow cytometry (Becton Dickinson, San Jose, Calif.). Upon analysis of the data, the anti-PTK7 antibodies have been categorized into 3 epitope groups—group A, which includes 7D11, group B, which includes 3G8 and 3G8a and group C, which includes 7C8, 12C6 and 12C6a.

### Example 5

### Characterization of Anti-PTK7 Antibody Binding to PTK7 Expressed on the Surface of Human Cancer Cells

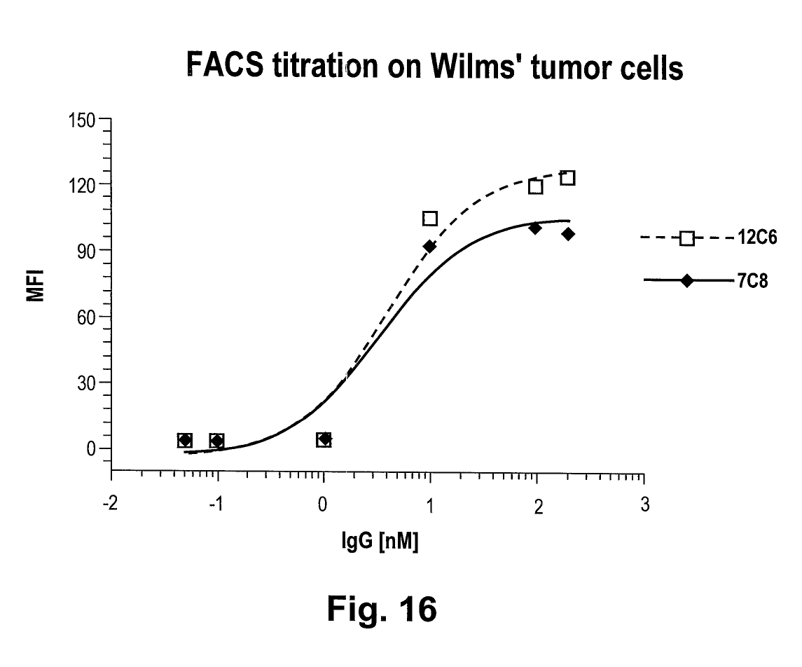

The nephroblastoma Wilms' tumor cell line G-401 (ATCC Acc No. CRL-1441) was tested for binding of the HuMAb anti-PTK7 human monoclonal antibodies 12C6 and 7C8 at different concentrations. Binding of the anti-PTK7 human monoclonal antibodies was assessed by incubating 1×10⁵ cells with antibody at a starting concentration of 30 μg/ml and serially diluting the antibody at a 1:10 dilution. The cells were washed and binding was detected with a PE-labeled anti-human IgG Ab. Flow cytometric analyses were performed using a FACScan flow cytometry (Becton Dickinson, San Jose, Calif.). The results are shown in FIG. 16. The anti-PTK7 monoclonal antibodies 12C6 and 7C8 bound to the nephroblastoma Wilms' tumor cell line in a concentration dependent manner, as measured by the mean fluorescent intensity (MFI) of staining. The EC₅₀ values for the anti-PTK7 monoclonal antibodies 12C6 and 7C8 was 4.0 nM and 3.4 nM, respectively.

These data demonstrate that the anti-PTK7 HuMAbs bind to kidney cancer cell lines.

### Example 6

### Binding of Human Anti-PTK7 Antibody to Cancer Cell Lines

Anti-PTK7 antibodies were tested for binding to a variety of cancer cell lines by flow cytometry.

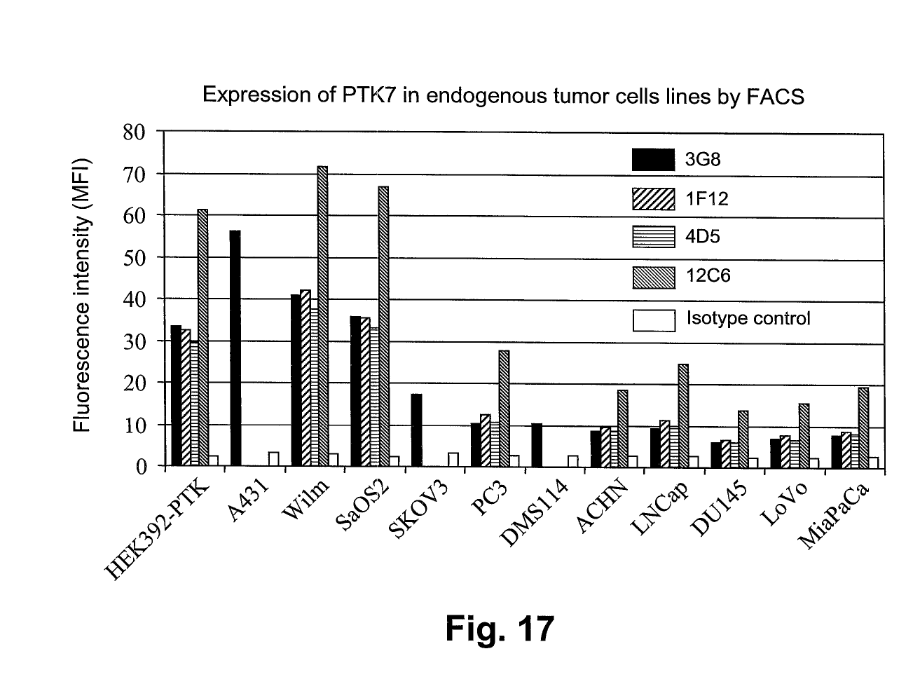

Binding of the 3G8, 12C6a, 4D5 and 12C6 anti-PTK7 human monoclonal antibodies to a panel of cancer cell lines was assessed by incubating cancer cell lines with anti-PTK7 human monoclonal antibodies at a concentration of 10 μg/ml. The cancer cell lines that were tested were A-431 (ATCC Acc No. CRL-1555), Wilms tumor cells G-401 (ATCC Acc No. CRL-1441), Saos-2 (ATCC Acc No. HTB-85), SKOV-3 (ATCC Acc No. HTB-77), PC3 (ATCC Acc No. CRL-1435), DMS 114 (ATCC Acc No. CRL-2066), ACHN (ATCC Ace No. CRL-1611), LNCaP (ATCC Acc No. CRL-1740), DU 145 (ATCC Acc No. HTB-81), LoVo (ATCC Acc No. CCL-229) and MIA PaCa-2 (ATCC Acc No. CRL-1420). An isotype control antibody was used as a negative control. The cells were washed and binding was detected with a FITC-labeled anti-human IgG Ab. Flow cytometric analyses were performed using a FACScan flow cytometry (Becton Dickinson, San Jose, Calif.). The results are shown in FIG. 17. The anti-PTK7 monoclonal antibodies 3G8, 12C6a, 4D5 and 12C6 bound to the cancer cell lines A-431, Wilms tumor cells G-401, Saos-2, SKOV-3, PC3, DMS 114, ACHN, LNCaP, DU 145, LoVo and MIA PaCa-2, as measured by the mean fluorescent intensity (MFI) of staining. These data demonstrate that the anti-PTK7 HuMAbs bind to a range of cancer cells that express cell surface PTK7.

### Example 7

### Binding of Anti-PTK7 to Human T, B and Dendritic Cells

Anti-PTK7 antibodies were tested for binding to CD4+, CD8+ T-cells, CD19+ B-cells and human blood myeloid dendritic cells expressing PTK7 on their cell surface by flow cytometry.

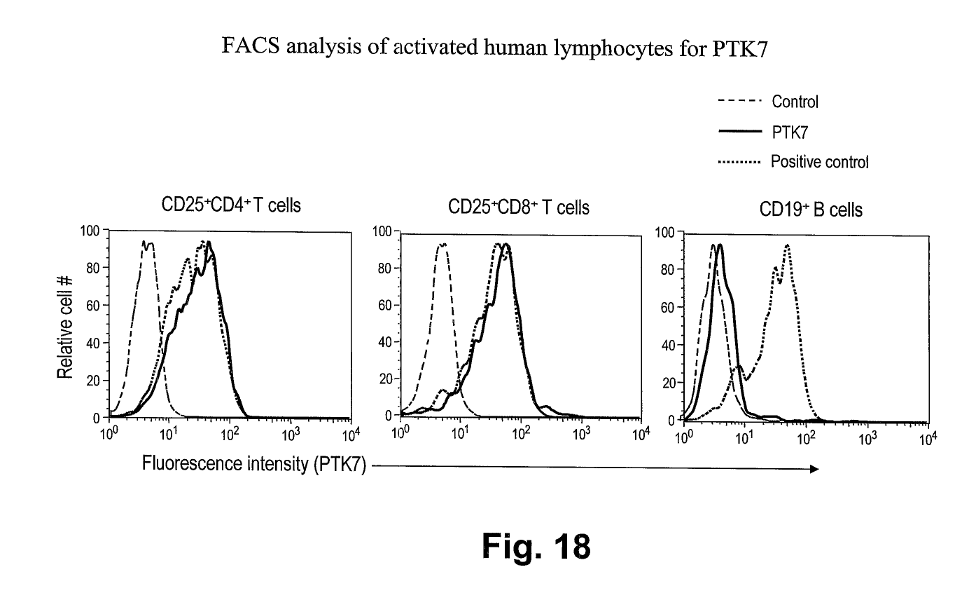

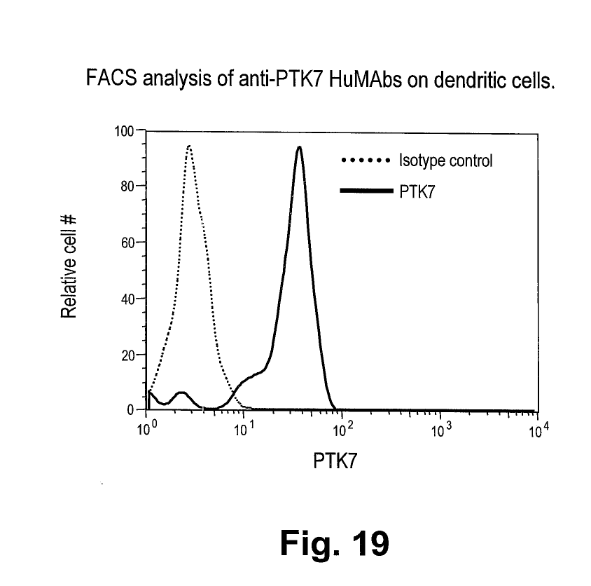

Human T cells were activated by anti-CD3 antibody to induce PTK7 expression on T cells prior to binding with a human anti-PTK7 monoclonal antibody. Binding of the 7c8 anti-PTK7 human monoclonal antibody was assessed by incubating the cells with anti-PTK7 human monoclonal antibodies at a concentration of 10 μg/ml. In some experiments, a known antibody that binds a T and B-cell specific marker was used as a positive control. The cells were washed and binding was detected with a FITC-labeled anti-human IgG Ab. Flow cytometric analyses were performed using a FACScan flow cytometry (Becton Dickinson, San Jose, Calif.). The results are shown in FIG. 18 (activated human T cells and B-cells) and 19 (dendritic cells). The anti-PTK7 monoclonal antibody 7C8 bound to activated human CD4+ and CD8+ T cells and dendritic cells, but not to B-cells, as measured by the mean fluorescent intensity (MFI) of staining. These data demonstrate that the anti-PTK7 HuMAbs bind to human T-cells and dendritic cells.

### Example 8

### Internalization of Anti-PTK7 Monoclonal Antibody

Anti-PTK7 HuMAbs were tested for the ability to internalize into PTK7-expressing cell lines using a Hum-Zap internalization assay. The Hum-Zap assay tests for internalization of a primary human antibody through binding of a secondary antibody with affinity for human IgG conjugated to the cytotoxin saporin.

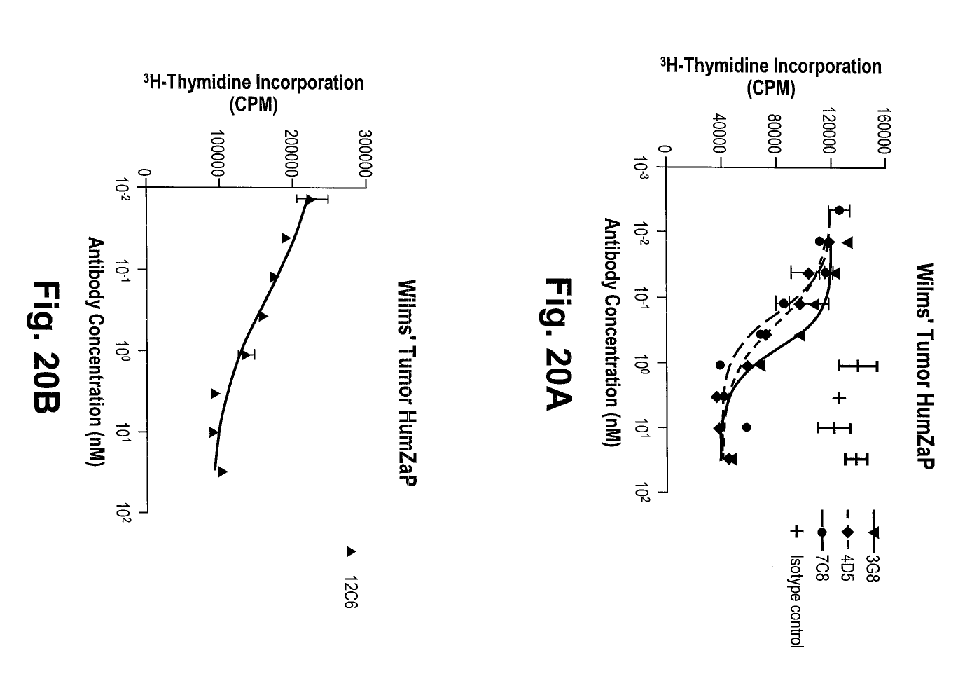

The PTK7-expressing cancer cell lines Wilms tumor G-401 (ATCC Acc No. CRL-1441), A-431 (ATCC Acc No. CRL-1555) and PC3 (ATCC Acc No. CRL-1435) were seeded at 1×10⁴ cells/well in 100 μl wells directly. The anti-PTK7 HuMAb antibodies 3G8, 4D5, 12C6 or 7C8 were added to the wells at a starting concentration of 30 nM and titrated down at 1:3 serial dilutions. An isotype control antibody that is non-specific for PTK7 was used as a negative control. The Hum-Zap (Advanced Targeting Systems, San Diego, Calif., IT-22-25) was added at a concentration of 11 nM and plates were allowed to incubate for 72 hours. The plates were then pulsed with 1.0 μCi of ³H-thymidine for 24 hours, harvested and read in a Top Count Scintillation Counter (Packard Instruments, Meriden, Conn.). The results are shown in FIGS. 20A-D. The anti-PTK7 antibodies 3G8, 4D5, 12C6 and 7C8 showed an antibody concentration dependent decrease in ³H-thymidine incorporation in the PTK7-expressing Wilms' Tumor cancer cell line. The anti-PTK7 antibodies 12C6 and 7C8 showed an antibody concentration dependent decrease in ³H-thymidine incorporation in the PTK7-expressing cancer cell lines A-431 and PC3. The EC₅₀ value for the anti-PTK7 antibodies 3G8, 4D5, 12C6 and 7C8 in Wilms' tumor cells was 0.6 nM, 0.3 nM, 0.2 nM and 0.2 nM, respectively. The EC₅₀ value for the anti-PTK7 antibodies 12C6 and 7C8 in A-431 cells was 0.2 nM and 0.2 nM, respectively. The EC₅₀ value for the anti-PTK7 antibodies 12C6 and 7C8 in PC3 tumor cells was 0.3 nM and 0.3 nM, respectively. This data demonstrates that the anti-PTK7 antibodies 3G8, 4D5, 12C6 and 7C8 internalize into cancer cells.

### Example 9

### Assessment of Cell Killing of a Cytotoxin-Conjugated Anti-PTK7 Antibody on Human Cancer Cell Lines

In this example, anti-PTK7 monoclonal antibodies conjugated to a cytotoxin were shown to kill PTK7+ human cancer cell lines in a cell proliferation assay. Anti-PTK7 antibodies may be conjugated to cytotoxins via a linker, such as a peptidyl, hydrazone or disulfide linker. Examples of cytotoxin compounds that may be conjugated to the antibodies of the current invention as well as linkers are described herein and in U.S. Application Ser. No. 60/720,499, filed on Sep. 26, 2005, incorporated herein by reference in its entirety.

The anti-PTK7 antibody 1F12 (SEQ ID NOS:84-98) was conjugated to formula (q), disclosed herein, to make 1F12-formula (q). The conjugation was performed as follows: The antibody at approximately 5 mg/ml in 100 mM Na-phosphate, 50 mM NaCl, 2 mM DTPA, pH 8.0, was thiolated with a 15-fold molar excess of 2-Iminothiolane for 1 hour at room temperature with continuous mixing. Following thiolation, the thiolated 1F12 was buffer exchanged into conjugation buffer (50 mM HEPES, 5 mM Glycine, 3% Glycerol, pH 6.0 by a PD10 column (Sephadex G-25) The concentration of the thiolated antibody and thiol concentration was determined. A 5 mM stock of formula (q) in DMSO was added at a 3-fold molar excess per thiol of antibody and mixed for 90 minutes at room temperature. The conjugated antibody was filtered through a 0.2 μM filter. The resulting conjugate was purified by size-exclusion chromatography on a Sephacryl-200 Size Exclusion column run in 50 mM HEPES, 5 mM glycine, 100 mM NaCl, pH 7.2. Fractions containing monomeric antibody conjugate were pooled and concentrated by ultrafiltration. Antibody conjugate concentration and substitution ratios were determined by measuring absorbance at 280 and 340 nm.

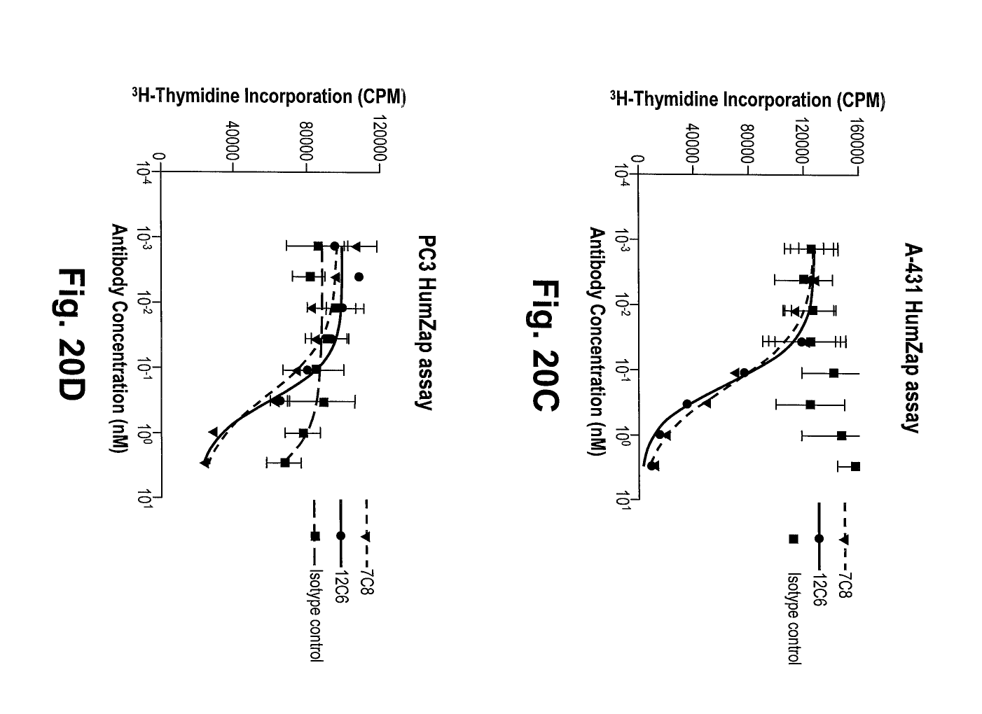

The PTK7-expressing Wilms' tumor human kidney cancer cell line G-401 (ATCC Acc No. CRL-1441) was seeded at 10⁴ cells/well in 100 μl wells for 3 hours. A 1F12-formula (p) was added to the wells at a starting concentration of 100 nM and titrated down at 1:3 serial dilutions. Plates were allowed to incubate for 48 hours. The plates were then pulsed with 1 μCi of ³H-thymidine for 24 hours before termination of the culture, harvested and read in a Top Count Scintillation Counter (Packard Instruments). FIG. 21 shows a decrease in ³H-thymidine incorporation into the PTK7-expressing Wilms' tumor human kidney cancer cell line with increasing concentrations of 1F12-formula (q). These data demonstrate that anti-PTK7 antibodies conjugated to cytotoxins show specific cytotoxicity to human kidney cancer cells.

### Example 10

### Assessment of Cell Killing of a Cytotoxin-Conjugated Anti-PTK7 Antibody on Human Tumor Cell Lines

In this example, anti-PTK7 monoclonal antibodies conjugated to a cytotoxin were shown to kill PTK7⁺ human tumor cell lines having either low, intermediate or high cell surface expression of PTK7 in a cell proliferation assay.

The anti-PTK7 HuMAb antibody 12C6a was conjugated to formula (p) resulting in the antibody conjugate referred to herein as 12C6a-formula (p). The conjugation of 12C6a to formula (p) was performed as follows: Approximately 5 mg/ml of 12C6a in 100 mM Na-phosphate, 50 mM NaCl, 2 mM DTPA, pH 8.0, was thiolated with a 15-fold molar excess of 2-Iminothiolane. The thiolation reaction was allowed to proceed for 1 hour at room temperature with continuous mixing. Following thiolation, the antibody was buffer exchanged into conjugation buffer (50 mM HEPES, 5 mM Glycine, 3% Glycerol, pH 6.0 by a PD10 column (Sephadex G-25) The concentration of the thiolated antibody and thiol concentration was determined.

A 5 mM stock of formula (p) in DMSO was then added at a 3-fold molar excess per thiol of antibody and mixed for 90 minutes at room temperature. The conjugated antibody was filtered through a 0.2 μm filter. The resulting conjugate was purified by size-exclusion chromatography on a Sephacryl-200 Size Exclusion column run in 50 mM HEPES, 5 mM glycine, 100 mM NaCl, pH 7.2. Fractions containing monomeric antibody conjugate were pooled and concentrated by ultrafiltration. Antibody conjugate concentration and substitution ratios were determined by measuring absorbance at 280 and 340 nm.

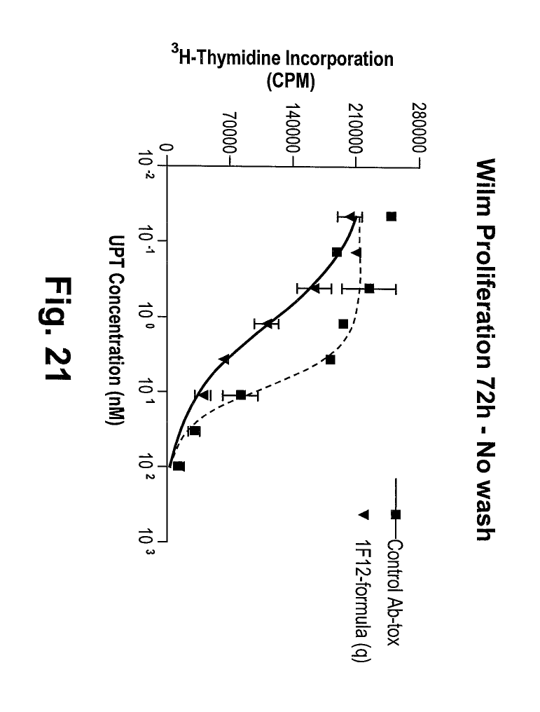

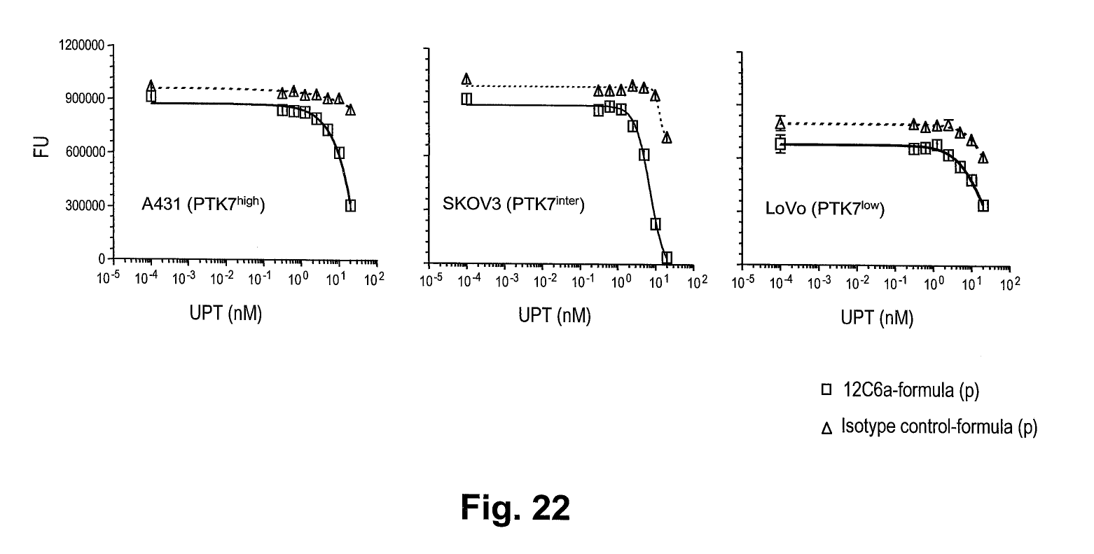

The PTK7-expressing human tumor cancer cell lines A-431, SKOV3, and LoVo were seeded at 10⁴ cells/well in 100 μl wells. The cell lines were previously tested for cell surface expression of PTK7 in a standard FACS assay. The A-431 cell line expressed the highest level of PTK7 cell surface expression and the LoVo cell line expressed the lowest level of PTK7 cell surface expression. 12C6a-formula (p) was added to the wells at a starting concentration of 20 nM and titrated down at 1:2 serial dilutions. An isotype control antibody was used as a negative control. The plates were incubated for 3 hours and the unbound (free) antibody-cytotoxin conjugates were washed out. The plates continued to incubate for 96 hrs and cell killing activity (FU, fluorescent unit) was measured using the CellTiter-Glo® Luminescent assay (Promega, WI, USA, Technical bulletin No. 288) and a BIO-TEK reader (Bio-Tek Instruments, Inc, VT, USA). The results are shown in FIG. 22. 12C6a-formula (p) showed a concentration-dependent decrease in living cells with all three cell lines, demonstrating that anti-PTK7 antibodies conjugated to a cytotoxin show specific cytotoxicity to various human cancer cells.

### Example 11

### Immunohistochemistry with 3G8, 12C6a, 2E11 and 7C8

The ability of the anti-PTK7 HuMAbs 3G8, 12C6a, 2E11 and 7C8 to recognize PTK7 by immunohistochemistry was examined using clinical biopsies from lung cancer, breast cancer, renal cancer, bladder cancer, pancreatic cancer, colon cancer, ovarian cancer, small intestine cancer, prostate cancer, melanoma, and cancers of the head and neck.

For immunohistochemistry, 5 μm frozen sections were used (Ardais Inc, USA). After drying for 30 minutes, sections were fixed with acetone (at room temperature for 10 minutes) and air-dried for 5 minutes. Slides were rinsed in PBS and then pre-incubated with 10% normal goat serum in PBS for 20 min and subsequently incubated with 10 μg/ml fitcylated 3G8, 12C6a or 2E11 antibody in PBS with 10% normal goat serum for 30 min at room temperature. Next, slides were washed three times with PBS and incubated for 30 min with mouse anti-FITC (10 μg/ml DAKO) at room temperature. Slides were washed again with PBS and incubated with Goat anti-mouse HRP conjugate (DAKO) for 30 minutes at room temperature. Slides were washed again 3× with PBS. Diaminobenzidine (Sigma) was used as substrate, resulting in brown staining. After washing with distilled water, slides were counter-stained with hematoxyllin for 1 min. Subsequently, slides were washed for 10 secs in running distilled water and mounted in glycergel (DAKO). Clinical biopsy immunohistochemical staining displayed positive staining in the lung cancer, breast cancer, bladder cancer, pancreatic cancer, colon cancer, ovarian cancer, small intestine cancer & prostate cancer sections. Normal tissue was always negative for PTK7 staining whereas within malignant tissue, both cancer activated fibroblasts and cancerous epithelial cells were observed to be positive for PTK7 staining. The identity of the cancer activated fibroblasts was confirmed in bladder cancer and breast cancer sections by staining with a Fibroblast Activation Protein antibody (FAP, Alexis Biochemicals, San Diego, USA). FAP is a known marker of cancer activated fibroblasts (Hofheinz et al. (2003) Oncologie 26:44-48).

7C8 was pre-complexed with a Fitc-labeled Goat anti Human Fab (Jackson #109-097-003) so that the final concentration of 7C8 was 5 □g/ml. This complex was then used with standard IHC methods to determine binding. 7C8 bound to ovarian cancers, pancreatic cancers, lung cancers (small cell and non small cell), melanomas, renal cancers, colon cancers, breast cancers, bladder cancers and cancers of the head and neck.

### Example 12

### Invasion Assay

In this example, antibodies directed against PTK7 were tested for the ability to affect cell invasion in a CHO cell line transfected with PTK7.

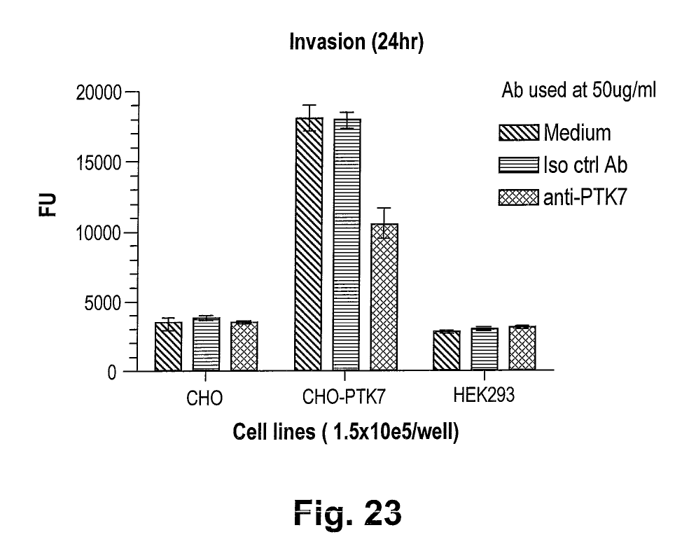

The assay was done using a HTS 96-Multiwell Insert System (Cat#351162, BD Biosciences, CA) according to the protocol. Either a CHO parent cell line, CHO cells transfected with full-length PTK7 or a control HEK293 cell line were mixed with either a pool of antiPTK7 HuMabs or an isotype control antibody prior to the addition of the cells into the inserts. The mixture (cells+Ab pool) was added into an insert well in the invasion plate. Following incubation at 37° C. with 5% CO2 for 24 hours, the cells were labeled with a fluorescent dye and cells that invaded to the bottom of the membrane were quantitated using a fluorescence plate reader. The results are shown in FIG. 23. This data demonstrates that anti-PTK7 antibodies inhibit the invasion mobility of cells expressing PTK7 on the cell surface.

### Example 13

### Treatment of In Vivo Pancreatic Cancer Cell Xenograft Model Using Unmodified and Cytotoxin-Conjugated Anti-PTK7 Antibodies

This Example shows that cytotoxin-conjugated anti-PTK7 antibodies inhibit tumor growth in mice implanted with a pancreatic cell carcinoma tumor. Examples of cytotoxin compounds that may be conjugated to the antibodies of the current invention were described in the pending U.S. patent application Ser. No. 11/134,826, incorporated herein by reference in its entirety. Two HuMAb anti-PTK7 antibody-toxin conjugates described herein were examined: 7C8-formula (o) and 7C8-formula (p).

Formula (p) was conjugated to 7C8 using the protocol described in Example 10 above. Formula (o) was conjugated to 7C8 as follows: Approximately 5 mg/ml of 7C8 in 100 mM Na-phosphate, 50 mM NaCl, 2 mM DTPA, pH 8.0, was thiolated with a 15-fold molar excess of 2-Iminothiolane for 1 hour at room temperature with continuous mixing. The antibody was then buffer exchanged into conjugation buffer (50 mM HEPES, 5 mM Glycine, 0.5% Povidone (10K) 2 mM DTPA, pH 5.5) by a PD10 column (Sephadex G-25). The concentration of the thiolated antibody and thiol concentration was determined. A 5 mM stock of formula (o) (r=4) in DMSO was added at a 3-fold molar excess per thiol of antibody and mixed for 90 minutes at room temperature. The conjugated antibody was filtered through a 0.2 μm filter. Following conjugation, 100 mM N-ethylmaleimide in DMSO was added at a 10-fold molar excess of thiol per antibody to quench any unreacted thiols. This quenching reaction was done for one hour at room temperature with continuous mixing. After incubation in the presence of NEM, the resulting conjugates were purified by size-exclusion chromatography on a Sephacryl-200 Size Exclusion column run in 50 mM HEPES, 5 mM glycine, 100 mM NaCl, pH 6.0. Fractions containing monomeric antibody conjugate were pooled and concentrated by ultrafiltration. Antibody conjugate concentration and substitution ratios were determined by measuring absorbance at 280 and 340 nm.

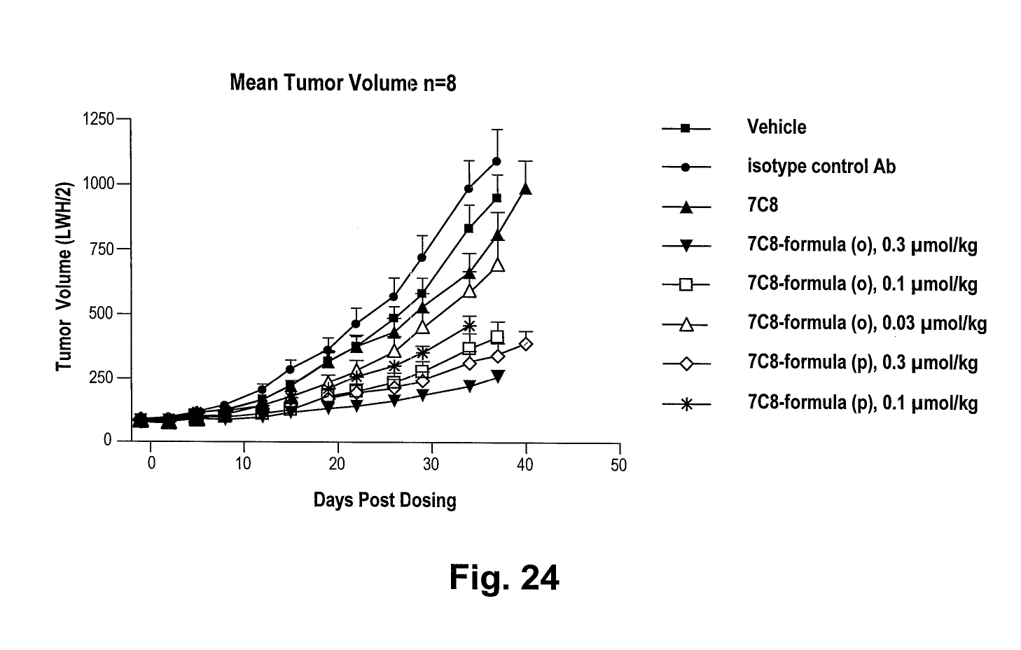

Many pancreatic cancer cell types may be used to show that anti-PTK7 antibody-toxin conjugates inhibit tumors. In this example, HPAC (human pancreatic adenocarcinoma, ATCC Accession Number CRL-2119) were chosen and expanded in vitro using standard laboratory procedures. Male Ncr athymic nude mice (Taconic, Hudson, N.Y.) between 6-8 weeks of age were implanted subcutaneously in the right flank with 2.5×10⁶ HPAC cells in 0.2 ml of PBS/Matrigel (1:1) per mouse. Mice were weighed and measured for tumors three dimensionally using an electronic caliper twice weekly after implantation. Tumor volumes were calculated as height×width×length/2. Mice with HPAC tumors averaging 90 mm³ were randomized into treatment groups. As shown in FIG. 24, on Day 0, the mice were administered a single intravenous dose of PBS vehicle, unmodified anti-PTK7 antibody, 7C8-formula (o), or 7C8-formula (p) at the indicated dosage (μmol/kg). Mice were monitored for tumor growth for 61 days post dosing. Mice were euthanized when the tumors reached tumor end point (2000 mm³) or ulcerated. 7C8 antibodies inhibited tumor growth progression with significantly increased inhibition demonstrated by the 7C8 conjugates (FIG. 24). The anti-tumor effects of the 7C8 conjugates were dose dependent, with the greatest effect seen at a dose of 0.3 μmol/kg. Treatment with the antibody conjugates was well tolerated, with subjects never experiencing greater than 5% median body weight loss (data not shown). Thus, treatment with an anti-PTK7 antibody-cytotoxin conjugate has a direct in vivo inhibitory effect on pancreatic cancer tumor growth.

### Example 14

### Treatment of In Vivo Breast Cancer Cell Xenograft Model Using Unmodified and Cytotoxin-Conjugated Anti-PTK7 Antibodies

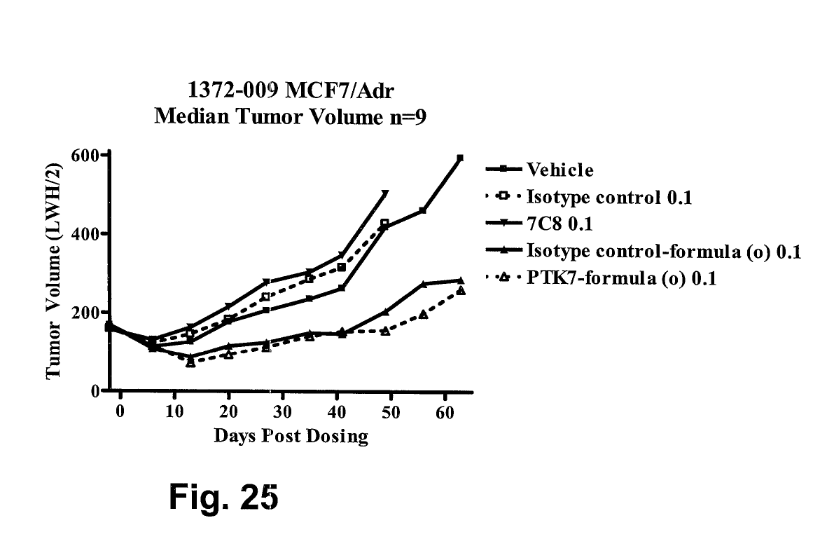

This Example shows that anti-PTK7 antibody conjugates inhibit the growth of breast cancer tumors in vivo. MCF7-adr cells, human breast cancer cells resistant to adriamycin, were expanded in vitro using standard laboratory procedures. Female CB17.SCID mice (Taconic, Hudson, N.Y.) between 6-8 weeks of age were implanted subcutaneously with 1.7 mg of 90-day release estrogen pellets, 3 0 mm size (Innovative Research of America, Sarasota, Fla.). The estrogen was administered in the neck region one day prior to being implanted subcutaneously in the right flank with 10×10⁶ MCF7-Adr cells in 0.2 ml of PBS/Matrigel (1:1) per mouse. Mice were weighed and measured for tumors three dimensionally using an electronic caliper twice weekly after implantation. Tumor volumes were calculated as height×width×length/2. Mice with MCF7-adr tumors averaging 160 mm³ were randomized into treatment groups. The mice were administered the PBS vehicle, a single intravenous dose at 0.1 μmol/kg of unmodified 7C8 or 7C8-formula (o) on Day 0. Mice were monitored for tumor growth for 63 days post dosing. Mice were euthanized when the tumors were ulcerated. The results are shown in FIG. 25. 7C8-formula (o) inhibited tumor growth. Thus, treatment with an anti-PTK7 antibody-cytotoxin conjugate has a direct in vivo inhibitory effect on breast cancer tumor growth.

### Example 15

### Tumor Inhibition In Vivo by a 7C8 Toxin Conjugate

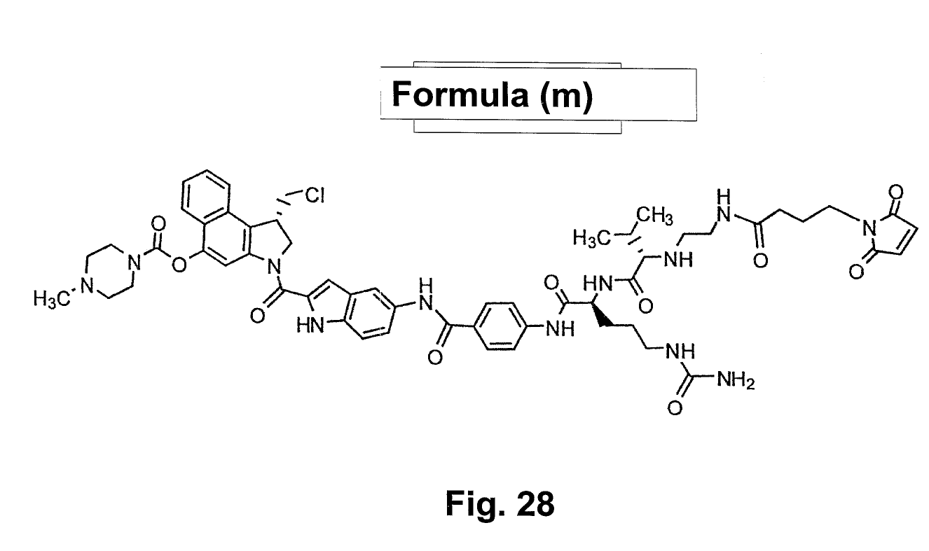

In this example, toxin conjugated 7C8 was shown to inhibit epithelial cell and lung tumor growth in in vivo SCID mouse models. In this example, 7C8 was conjugated to formula (m). The structure of formula (m) is shown in FIG. 28. Formula (m), and preparation thereof, is described further in U.S. Application Ser. No. 60/882,461, filed Dec. 28, 2006, the entire content of which is specifically incorporated herein by reference. The 7C8-formula (m) conjugate was prepared as follows:

Anti-PTK7 antibody 7C8 was concentrated to approximately 5 mg/ml, buffer exchanged into 100 mM phosphate buffer, 50 mM NaCl, 2 mM DTPA pH 8.0 and thiolated by addition of an 8 to 10-fold molar excess of 2-Imminothiolane for 60 minutes at room temperature. Following thiolation, the antibody was buffer exchanged into 50 mM HEPES buffer, containing 300 mM glycine, 2 mM DTPA, and 0.5% Povidone (10 K) pH 5.5. Thiolation was quantified with 4,4″-dithiodipyridine by measuring thiopyridine release at 324 nM. Conjugation was achieved by addition of maleimide containing formula (m) at a 3:1 molar ratio of drug to thiols. Incubation was at room temperature for 60 minutes followed by the addition of 10:1 molar ratio of N-ethylmaleimide (NEM) to thiols to the reaction mix to quench any unreacted thiols. This quenching reaction was done for one hour at room temperature with continuous mixing.

The antibody drug conjugate was then 0.2 μm filtered prior to Cation-exchange chromatographic purification. The SP Sepharose High Performance Cation Exchange column (CEX) was regenerated with 5 CV (column volume) of 50 mM HEPES, 5 mM Glycine, 1M NaCl, pH 5.5. Following regeneration, the column was equilibrated with 3 CVs of equilibration buffer (50 mM HEPES, 5 mM Glycine, pH 5.5). The 7C8-formula (m) conjugate was loaded and the column was washed once with the equilibration buffer. The conjugate was eluted with 50 mM HEPES, 5 mM Glycine, 230 mM NaCl, pH 5.5. Eluate was collected in fractions. The column was then regenerated with 50 mM HEPES, 5 mM Glycine, 1M NaCl, pH 5.5 to remove protein aggregates and any unreacted formula (m). Pooling of eluate fractions was based on aggregation levels and Substitution Ratios (SR), i.e., mole of Drug per mole of Antibody. The pooling criteria was >95% monomer determined by SEC-HPLC with an SR range of 1-2. The purified CEX eluate pool was buffer exchanged into bulk diafiltration buffer (30 mg/ml Sucrose, 10 mg/ml Glycine, pH 6.0) in a 10 NMWCO flat-sheet TFF cassette with a PES membrane. Bulk formulation was completed by dilution of the protein concentration to 5 mg/ml and by the addition of Dextran 40 to the diafiltered conjugate solution to a final concentration of 10 mg/ml. The formulated bulk was filtered through a 0.2 μm PES filter into sterile PETG bottles and stored at 2° C. to 8° C.

The effect of 7C8 and 7C8-formula (m) on the growth of A431 xenograft tumors in an in vivo murine model was then examined. A431 is an epithelial cell line that expresses PTK-7 and is thus representative of epithelial cancers that express the PTK-7 protein, including: breast cancer, colon cancer, lung cancer, stomach cancer, renal cancer, head and neck cancer, bladder cancer, prostate cancer, and pancreatic cancer. Moreover, 7C8, has been shown by FACS and IHC to bind cell lines and cancers representing these diseases. PTK7 is sometimes also expressed on the activated stroma of epithelial cancers. Anti-PTK7 antibody drug conjugates such as 7C8-formula (m) would have anti-cancer activity in these cancers. This is similar to the activity of anti-RG1 toxin conjugates. RG-1 is also expressed on cellular stroma. The anti-cancer activity of anti-RG-1 antibody drug conjugates in xenograft models of prostate cancer is described in U.S. Patent Application No. 60/991,690.

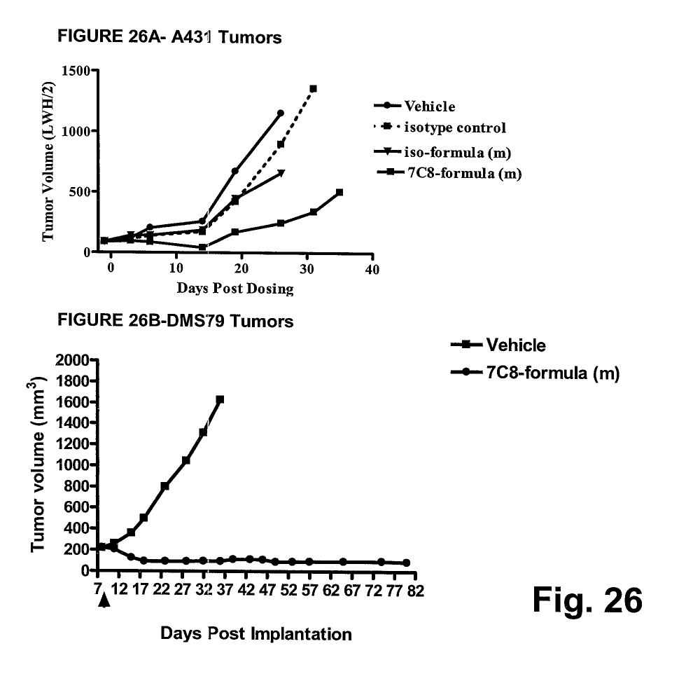

In the A431 xenograft model, SCID mice (CB17 SCID, Taconic Farms, Germantown, N.Y.) were implanted with A431 cells and allowed to grow until the tumor reached approximately 90 mm³. The mice were then randomized and treated intraperitoneally with a single dose of vehicle alone, 0.3 μmole/kg of an isotype-matched human IgG antibody linked to formula (m) (iso-formula (m)), unmodified 7C8, or with 7C8-formula (m) conjugate (0.3 μmole/kg). Tumor volume was measured at regular intervals beyond 35 days (FIG. 26).

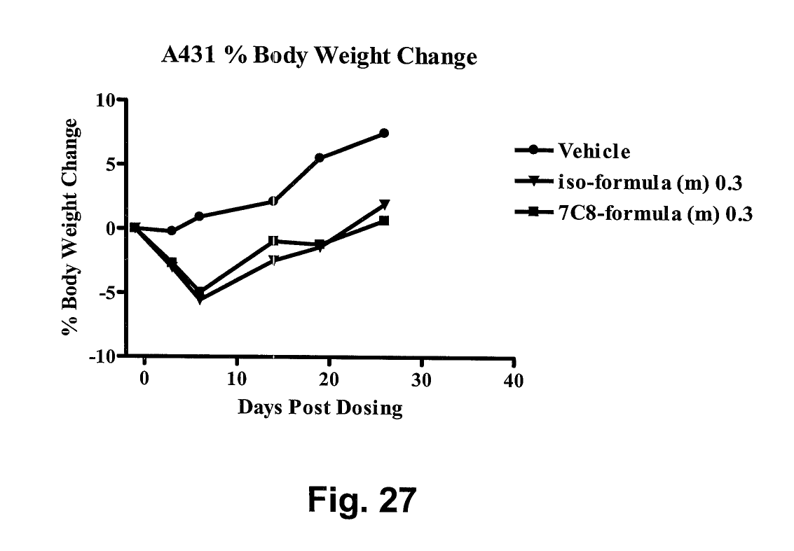

While treatment with the unmodified 7C8 antibody or isotype matched-formula (m) antibody did not show an effect on A431 tumor cell volume (i.e., did not inhibit tumor growth), treatment with the 7C8-formula (m) conjugate significantly inhibited tumor growth, as illustrated in FIG. 26A. Additionally, toxicity was not associated with the 7C8-formula (m) conjugate when administered to mice as measured by body weight (FIG. 27).

In a second set of analyses, a 7C8-formula (m) conjugate inhibited the growth of small cell lung cancer derived DMS79 cells in the mouse xenograft model. In the xenograft model, SCID mice were implanted with 5×10⁶ DMS79 cells per mouse and allowed to grow until the mean tumor volume was ca. 200 mm3. The mice were then randomized and treated intraperitoneally with 7C8-formula (m) conjugate (0.3 mmol/kg). As shown in FIG. 27C, treatment with the 7C8-formula (m) conjugate caused complete tumor regression in all mice through day 81.

In summary, an anti-PTK7 antibody toxin conjugate significantly inhibited epithelial and lung tumor growth in vivo and did not show significant toxicity in mice.

### Example 16

### Assessment of Toxicity of 7C8-formula (m) in Cynomolgus Monkeys

Cynomolgus monkeys and humans show similar patterns of PTK7 expression. Immunohistochemistry analyses show that 7C8 binds to the same tissues in cynomolgus monkeys as it does in humans. Thus, cynomolgus monkeys are suitable to assess the on target toxicities of 7C8-formula (m).

7C8-formula (m) was administered intravenously to two male and two female cynomolgus monkeys. Two doses of 0.4 μmol/kg were given on days 1 and 15. The animals were observed for behavioural changes, signs of toxicity, and blood was drawn for analysis. No behavioural changes were noted. Blood cell and chemistry analyses revealed no drug related changes. Pathological examination of tissues known to express ptk7 (e.g. ovarian fibroblasts) showed no evidence of toxicities induced by 7C8-formula (m). Thus, 7C8-formula (m) toxicity was not detected in cynomolgus monkeys.

| SEQ ID | | SEQ ID | |
|---|---|---|---|
| NO: | SEQUENCE | NO: | SEQUENCE |
| 1 | VH a.a. 3G8, 3G8a | 22 | VH CDR3 a.a. 7C8 |
| 2 | VH a.a. 4D5 | 23 | VK CDR1 a.a. 3G8 |
| 3 | VH a.a. 12C6, 12C6a | 24 | VK CDR1 a.a. 3G8a |
| 4 | VH a.a. 7C8 | 25 | VK CDR1 a.a. 4D5 |
| 5 | VK a.a. 3G8 | 26 | VK CDR1 a.a. 12C6 |
| 6 | VK a.a. 3G8a | 27 | VK CDR1 a.a. 12C6a |
| 7 | VK a.a. 4D5 | 28 | VK CDR1 aa. 7C8 |
| 8 | VK a.a. 12C6 | 29 | VK CDR2 a.a. 3G8 |
| 9 | VK a.a. 12C6a | 30 | VK CDR2 a.a. 3G8a |
| 10 | VK a.a. 7C8 | 31 | VK CDR2 a.a. 4D5 |
| 11 | VH CDR1 a.a. 3G8 | 32 | VK CDR2 a.a. 12C6 |
| 12 | VH CDR1 a.a. 4D5 | 33 | VK CDR2 a.a. 12C6a |
| 13 | VH CDR1 a.a. 12C6 | 34 | VK CDR2 a.a. 7C8 |
| 14 | VH CDR1 a.a. 7C8 | 35 | VK CDR3 a.a. 3G8 |
| 15 | VH CDR2 a.a. 3G8 | 36 | VK CDR3 a.a. 3G8a |
| 16 | VH CDR2 a.a. 4D5 | 37 | VK CDR3 a.a. 4D5 |
| 17 | VH CDR2 a.a. 12C6 | 38 | VK CDR3 a.a. 12C6 |
| 18 | VH CDR2 a.a. 7C8 | 39 | VK CDR3 a.a. 12C6a |
| 19 | VH CDR3 a.a. 3G8 | 40 | VK CDR3 a.a. 7C8 |
| 20 | VH CDR3 a.a. 4D5 | 41 | VH n.t. 3G8, 3G8a |
| 21 | VH CDR3 a.a. 12C6 | 42 | VH n.t. 4D5 |
| 43 | VH n.t. 12C6, 12C6a | 71 | Peptide Linker |
| 44 | VH n.t. 7C8 | 72 | Peptide Linker |
| 45 | VK n.t. 3G8 | 73 | Peptide Linker |
| 46 | VK n.t. 3G8a | 74 | Peptide Linker |
| 47 | VK n.t. 4D5 | 75 | Peptide Linker |
| 48 | VK n.t. 12C6 | 76 | Peptide Linker |
| 49 | VK n.t. 12C6a | 77 | Peptide Linker |
| 50 | VK n.t. 7C8 | 78 | Peptide Linker |
| 51 | VH 3-30.3 germline a.a. | 79 | Peptide Linker |
| 52 | VH DP44 germline a.a. | 80 | Peptide Linker |
| 53 | VH 3-33 germline a.a. | 81 | Peptide Linker |
| 54 | VK L15 germline a.a. | 82 | Peptide Linker |
| 55 | VK A10 germline a.a. | 83 | Peptide Linker |
| 56 | VK A27 germline a.a. | 84 | VH a.a. 1F12 |
| 57 | VK L6 germline a.a. | 85 | VK1 a.a. 1F12 |
| 58 | PTK7 a.a. | 86 | VK2 a.a. 1F12 |
| 59 | JH4b germline a.a | 87 | VH CDR1 a.a. 1F12 |
| 60 | JH4b germline a.a. | 88 | VH CDR2 a.a. 1F12 |
| 61 | 3-7 germline a.a. | 89 | VH CDR3 a.a. 1F12 |
| 62 | 3-23 germline a.a. | 90 | VK1 CDR1 a.a. 1F12 |
| 63 | JH4b germline a.a | 91 | VK1 CDR2 a.a. 1F12 |
| 64 | JH6b germline a.a. | 92 | VK1 CDR3 a.a. 1F12 |
| 65 | JK1 germline a.a. | 93 | VK2 CDR1 a.a. 1F12 |
| 66 | JK5 germline a.a. | 94 | VK2 CDR2 a.a. 1F12 |
| 67 | JK2 germline a.a. | 95 | VK2 CDR3 a.a. 1F12 |
| 68 | JK2 germline a.a. | 96 | VH n.t. 1F12 |
| 69 | JK3 germline a.a. | 97 | VK1 n.t. 1F12 |
| 70 | Peptide Linker | 98 | VK2 n.t. 1F12 |

1-17. (canceled)

18. An antibody-partner molecule conjugate comprising an antibody, or an antigen-binding portion thereof, that specifically binds PTK-7, and a partner molecule, wherein the partner molecule is selected from the group consisting of:

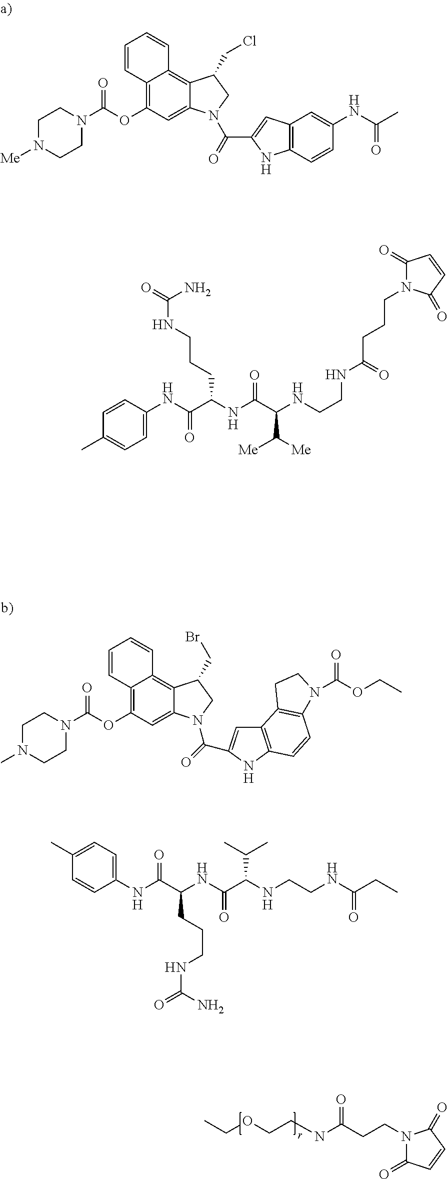

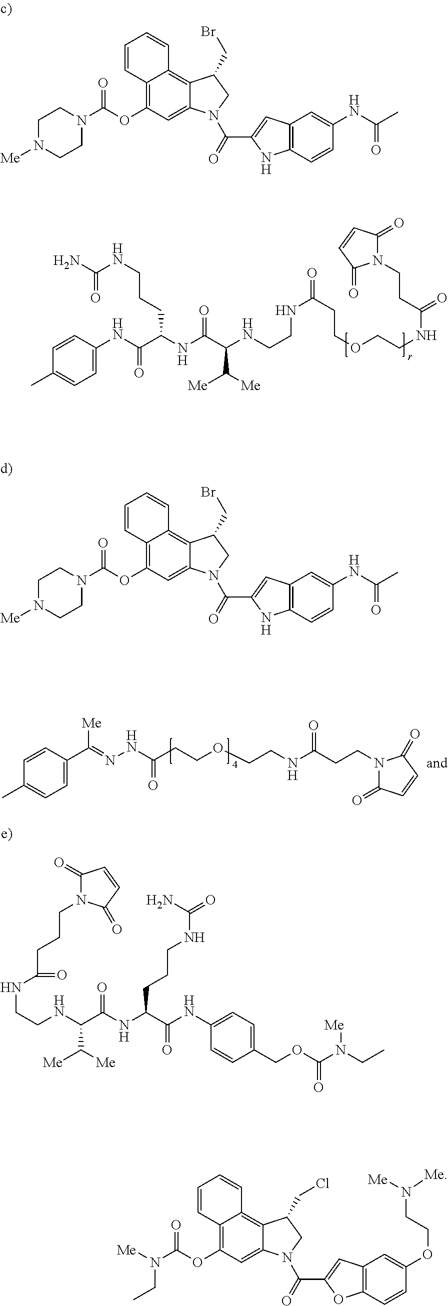

19. The antibody-partner molecule conjugate of claim 18, wherein the antibody, or antigen binding portion thereof, competes for binding to PTK-7 with an antibody comprising the amino acid sequences set forth in SEQ ID NOs:2 and 7, SEQ ID NOs:4 and 10, SEQ ID NOs:1 and 5, SEQ ID NOs:1 and 6, SEQ ID NOs:3 and 8, SEQ ID NOs:3 and 9, SEQ ID NOs:84 and 85, or SEQ ID NOs:84 and 86, respectively.

20. The antibody-partner molecule conjugate of claim 18, wherein the antibody, or antigen binding portion thereof, binds to an epitope on PTK-7 recognized by an antibody comprising the amino acid sequences set forth in SEQ ID NOs:2 and 7, SEQ ID NOs:4 and 10, SEQ ID NOs:1 and 5, SEQ ID NOs:1 and 6, SEQ ID NOs:3 and 8, SEQ ID NOs:3 and 9, SEQ ID NOs:84 and 85, or SEQ ID NOs:84 and 86, respectively.

21. The antibody-partner molecule conjugate of claim 18, wherein the antibody, or antigen-binding portion thereof, comprises heavy and light chain variable regions comprising the amino acid sequences set forth in SEQ ID NOs:2 and 7, SEQ ID NOs:4 and 10, SEQ ID NOs:1 and 5, SEQ ID NOs:1 and 6, SEQ ID NOs:3 and 8, SEQ ID NOs:3 and 9, SEQ ID NOs:84 and 85, or SEQ ID NOs:84 and 86, respectively.

22. The antibody-partner molecule conjugate of claim 18, wherein the antibody, or antigen-binding portion thereof, comprises:
(a) a heavy chain variable region CDR1 comprising SEQ ID NO:12, a heavy chain variable region CDR2 comprising SEQ ID NO:16, a heavy chain variable region CDR3 comprising SEQ ID NO:20, a light chain variable region CDR1 comprising SEQ ID NO:25, a light chain variable region CDR2 comprising SEQ ID NO:31; and a light chain variable region CDR3 comprising SEQ ID NO:37;
(b) a heavy chain variable region CDR1 comprising SEQ ID NO:14, a heavy chain variable region CDR2 comprising SEQ ID NO:18, a heavy chain variable region CDR3 comprising SEQ ID NO:22, a light chain variable region CDR1 comprising SEQ ID NO:28, a light chain variable region CDR2 comprising SEQ ID NO:34; and a light chain variable region CDR3 comprising SEQ ID NO:40;
(c) a heavy chain variable region CDR1 comprising SEQ ID NO:11, a heavy chain variable region CDR2 comprising SEQ ID NO:15, a heavy chain variable region CDR3 comprising SEQ ID NO:19, a light chain variable region CDR1 comprising SEQ ID NO:23, a light chain variable region CDR2 comprising SEQ ID NO:29; and a light chain variable region CDR3 comprising SEQ ID NO:35;
(d) a heavy chain variable region CDR1 comprising SEQ ID NO:11, a heavy chain variable region CDR2 comprising SEQ ID NO:15, a heavy chain variable region CDR3 comprising SEQ ID NO:19, a light chain variable region CDR1 comprising SEQ ID NO:24, a light chain variable region CDR2 comprising SEQ ID NO:30; and a light chain variable region CDR3 comprising SEQ ID NO:36;
(e) a heavy chain variable region CDR1 comprising SEQ ID NO:13, a heavy chain variable region CDR2 comprising SEQ ID NO:17, a heavy chain variable region CDR3 comprising SEQ ID NO:21, a light chain variable region CDR1 comprising SEQ ID NO:26, a light chain variable region CDR2 comprising SEQ ID NO:32; and a light chain variable region CDR3 comprising SEQ ID NO:38; or
(f) a heavy chain variable region CDR1 comprising SEQ ID NO:13, a heavy chain variable region CDR2 comprising SEQ ID NO:17, a heavy chain variable region CDR3 comprising SEQ ID NO:21, a light chain variable region CDR1 comprising SEQ ID NO:27, a light chain variable region CDR2 comprising SEQ ID NO:33; and a light chain variable region CDR3 comprising SEQ ID NO:39.

23. An antibody-partner molecule conjugate comprising an antibody, or an antigen-binding portion thereof, that specifically binds PTK-7, and a partner molecule, wherein the antibody comprises heavy and light chain variable regions comprising the amino acid sequences set forth in SEQ ID NOs:2 and 7, respectively, and the partner molecule is:

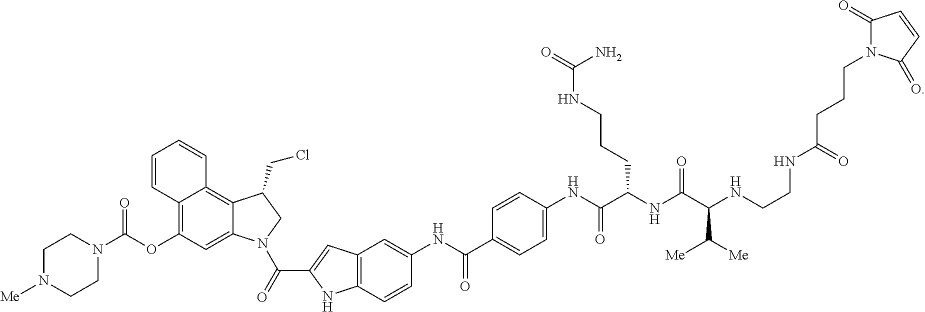

24. An antibody-partner molecule conjugate comprising an antibody, or an antigen-binding portion thereof, wherein the antibody, or antigen binding portion thereof, comprises the amino acid sequences set forth in SEQ ID NOs: 2 and 7, respectively.

25. The antibody-partner molecule conjugate of claim 18, wherein the antibody-partner molecule conjugate is conjugated to the antibody by a linker selected from the group consisting of thiol linkers, peptidyl linkers, hydrazine linkers, and disulfide linkers.

26. A composition comprising the antibody-partner molecule conjugate of claim 18, and a pharmaceutically acceptable carrier.

27. An isolated nucleic acid molecule encoding the antibody or antigen-binding portion thereof, of claim 21 or 22.

28. An expression vector comprising the nucleic acid molecule of claim 27.

29. A host cell comprising the expression vector of claim 28.

30. A method of treating or preventing a disease characterized by growth of tumor cells expressing PTK7, comprising administering to a subject the antibody-partner molecule conjugate of claim 18, in an amount effective to treat or prevent the disease.

31. The method of claim 30, wherein the disease is cancer.

32. The method of claim 31, wherein the cancer is selected from the group consisting of colon cancer, lung cancer, breast cancer, pancreatic cancer, melanoma, acute myeloid leukemia, kidney cancer, bladder cancer, ovarian cancer, prostate cancer, and an epithelial cell cancer.


SEQUENCE LISTING (80 sequences)

SEQ ID NO: 1 - protein, 116 aa, Homo sapiens
QVQLVESGGGVVQPGRSLRLSCAASGFIFSNYAMHWVRQAPGKGLEWVAVISYDGNNKYYADSVKGRFTISRDNSKNTLYLQMNSLRAEDTAVYYCAREVWSIDNWGQGTLVTVSS

SEQ ID NO: 2 - protein, 115 aa, Homo sapiens
QVQLVESGGGVVQPGRSLRLSCAASGFTFSSYAFHWVRQAPGKGLEWVAVISYDGSIKYYADSVKGRFTISRDNSKNTLYLQMNSLRAEDTAVYYCARTYYFDYWGQGTLVTVSS

SEQ ID NO: 3 - protein, 112 aa, Homo sapiens
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 4 - protein, 126 aa, Homo sapiens
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 5 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 6 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPFTFGPGTKVDIK

SEQ ID NO: 7 - protein, 107 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPITFGQGTRLEIK

SEQ ID NO: 8 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 9 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 10 - protein, 108 aa, Homo sapiens
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 11 - protein, 5 aa, Homo sapiens
NYAMH

SEQ ID NO: 12 - protein, 5 aa, Homo sapiens
SYAFH

SEQ ID NO: 13 - protein, 5 aa, Homo sapiens
TYLMY

SEQ ID NO: 14 - protein, 5 aa, Homo sapiens
SYGMH

SEQ ID NO: 15 - protein, 17 aa, Homo sapiens
VISYDGNNKYYADSVKG

SEQ ID NO: 16 - protein, 17 aa, Homo sapiens
VISYDGSIKYYADSVKG

SEQ ID NO: 17 - protein, 16 aa, Homo sapiens
AIGSGGDTYYADSVKG

SEQ ID NO: 18 - protein, 17 aa, Homo sapiens
VIWDDGSNKYYVDSVKG

SEQ ID NO: 19 - protein, 7 aa, Homo sapiens
EVWSIDN

SEQ ID NO: 20 - protein, 6 aa, Homo sapiens
TYYFDY

SEQ ID NO: 21 - protein, 4 aa, Homo sapiens
GLGY

SEQ ID NO: 22 - protein, 17 aa, Homo sapiens
DDYYGSGSFNSYYGTDV

SEQ ID NO: 23 - protein, 11 aa, Homo sapiens
RASQGISSWLA

SEQ ID NO: 24 - protein, 11 aa, Homo sapiens
RASQGISSWLA

SEQ ID NO: 25 - protein, 11 aa, Homo sapiens
RASQSIGSSLH

SEQ ID NO: 26 - protein, 12 aa, Homo sapiens
RASQSVSSSYLA

SEQ ID NO: 27 - protein, 11 aa, Homo sapiens
RASQGISSWLA

SEQ ID NO: 28 - protein, 11 aa, Homo sapiens
RASQSVSIYLA

SEQ ID NO: 29 - protein, 7 aa, Homo sapiens
AASSLQS

SEQ ID NO: 30 - protein, 7 aa, Homo sapiens
AASSLQS

SEQ ID NO: 31 - protein, 7 aa, Homo sapiens
YASQSFS

SEQ ID NO: 32 - protein, 7 aa, Homo sapiens
GASSRAT

SEQ ID NO: 33 - protein, 7 aa, Homo sapiens
AASSLQS

SEQ ID NO: 34 - protein, 7 aa, Homo sapiens
DASNRAT

SEQ ID NO: 35 - protein, 9 aa, Homo sapiens
QQYNSYPRT

SEQ ID NO: 36 - protein, 9 aa, Homo sapiens
QQYNSYPFT

SEQ ID NO: 37 - protein, 9 aa, Homo sapiens
HQSSSLPIT

SEQ ID NO: 38 - protein, 10 aa, Homo sapiens
QQYGSSPMYT

SEQ ID NO: 39 - protein, 9 aa, Homo sapiens
QQYNSYPYT

SEQ ID NO: 40 - protein, 10 aa, Homo sapiens
QQRSNWPPFT

SEQ ID NO: 41 - DNA, 348 bp, Homo sapiens
caggtgcagctggtggagtctgggggaggcgtggtccagcctgggaggtccctgcgactctcctgtgcagcctctggattcatcttcagtaactatgctatgcactgggtccgccaggctccaggcaaggggctggagtgggtggcagttatatcatatgatggaaacaataaatactacgcagactccgtgaagggccgattcaccatctccagagacaattccaagaacacgctgtatctgcaaatgaacagcctgagagctgaggacacggctgtgtattactgtgcgagagaggtctggagtattgacaactggggccagggaaccctggtcaccgtctcctca

SEQ ID NO: 42 - DNA, 345 bp, Homo sapiens
caggtgcagctggtggagtctgggggaggcgtggtccagcctgggaggtccctgagactctcctgtgcagcctctggattcaccttcagtagctatgctttccactgggtccgccaggctccaggcaaggggctggagtgggtggcagttatatcatatgatggaagcattaaatactacgcagactccgtgaagggccgattcaccatctccagagacaattccaagaacacgctgtatctgcaaatgaacagcctgagagctgaggacacggctgtgtattactgtgcgaggacgtactactttgactactggggccagggaaccctggtcaccgtctcctca

SEQ ID NO: 43 - DNA, 336 bp, Homo sapiens
gaggttcagctggtgcagtctgggggaggcttggtacatcctggggggtccctgagactctcctgtgcaggctctggattcaccttcagtacctatcttatgtactgggttcgccaggctccaggaaaaactctggagtgggtctcagctattggttctggtggtgatacatactatgcagactccgtgaagggccgattcaccatctccagagacaatgccaagaactccttgtatcttcaaatgaacagcctgagagccgaggacatggctgtgtattactgtgcaagaggactgggctactggggccagggaaccctggtcaccgtctcctca

SEQ ID NO: 44 - DNA, 378 bp, Homo sapiens
caggtgcaactggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatgggatgatggaagtaataaatactatgtagactccgtgaagggccgattcaccatctccagagacaattccaagaacacgctgtatctgcaaatgaacagcctgagagccgaggacacggctgtgtattactgtgcgagagatgattactatggttcggggagttttaactcctactacggtacggacgtctggggccaagggaccacggtcaccgtctcctca

SEQ ID NO: 45 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctcggacgttcggccaagggaccaaggtggaaatcaaa

SEQ ID NO: 46 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccattcactttcggccctgggaccaaagtggatatcaaa

SEQ ID NO: 47 - DNA, 321 bp, Homo sapiens
gaaattgtgctgactcagtctccagactttcagtctgtgactccaaaggagaaagtcaccatcacctgccgggccagtcagagcattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcagcgtattactgtcatcagagtagtagtttaccgatcaccttcggccaagggacacgactggagattaaa

SEQ ID NO: 48 - DNA, 327 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccatgtacacttttggccaggggaccaagctggagatcaaa

SEQ ID NO: 49 - DNA, 321 bp, Homo sapiens
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtacacttttggccaggggaccaagctggagatcaaa

SEQ ID NO: 50 - DNA, 324 bp, Homo sapiens
gaaattgtgttgacacagtctccagccaccctgtctttgtctccaggggaaagagccaccctctcctgcagggccagtcagagtgttagcatctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccattcactttcggccctgggaccaaagtggatatcaaa

SEQ ID NO: 51 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 52 - protein, 97 aa, Homo sapiens
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 53 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 54 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 55 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 56 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 57 - protein, 96 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPP

SEQ ID NO: 58 - protein, 1070 aa, Homo sapiens
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 59 - protein, 13 aa, Homo sapiens
DYWGQGTLVTVSS

SEQ ID NO: 60 - protein, 15 aa, Homo sapiens
YFDYWGQGTLVTVSS

SEQ ID NO: 61 - protein, 13 aa, Homo sapiens
ANAKQDGSEKYYV

SEQ ID NO: 62 - protein, 6 aa, Homo sapiens
SGSGGS

SEQ ID NO: 63 - protein, 12 aa, Homo sapiens
YWGQGTLVTVSS

SEQ ID NO: 64 - protein, 18 aa, Homo sapiens
YYYGMDVWGQGTTVTVSS

SEQ ID NO: 65 - protein, 11 aa, Homo sapiens
TFGQGTKVEIK

SEQ ID NO: 66 - protein, 12 aa, Homo sapiens
ITFGQGTRLEIK

SEQ ID NO: 67 - protein, 12 aa, Homo sapiens
YTFGQGTKLEIK

SEQ ID NO: 68 - protein, 12 aa, Homo sapiens
YTFGQGTKLEIK

SEQ ID NO: 69 - protein, 12 aa, Homo sapiens
FTFGPGTKVDIK

SEQ ID NO: 70 - protein, 4 aa, Artificial
  Peptide linker
XLAL

SEQ ID NO: 71 - protein, 4 aa, Artificial
  Peptide linker
XLAL

SEQ ID NO: 72 - protein, 6 aa, Artificial
  Peptide linker
PVGLIG

SEQ ID NO: 73 - protein, 5 aa, Artificial
  Peptide linker
GPLGV

SEQ ID NO: 74 - protein, 8 aa, Artificial
  Peptide linker
GPLGIAGQ

SEQ ID NO: 75 - protein, 4 aa, Artificial
  Peptide linker
PLGL

SEQ ID NO: 76 - protein, 8 aa, Artificial
  Peptide linker
GPLGMLSQ

SEQ ID NO: 77 - protein, 8 aa, Artificial
  Peptide linker
GPLGLWAQ

SEQ ID NO: 78 - protein, 4 aa, Artificial
  Peptide linker
ALAL

SEQ ID NO: 79 - protein, 4 aa, Artificial
  Peptide linker
GFLG

SEQ ID NO: 80 - protein, 4 aa, Artificial
  Peptide linker
LLGL